SEQUENCE LISTING (140 sequences)

SEQ ID NO: 1 - DNA, 157 bp, Homo sapiens
gttcctcaggccagatctgacccactggcatttattacattttctgctaaaggagccactctcaacctggaagagaggagatctgtggcaatcagatccagagagaatgtggtcttcgtacagactgataaacccacctacaagcctggacagaaag

SEQ ID NO: 2 - protein, 53 aa, Homo sapiens
VPQARSDPLAFITFSAKGATLNLEERRSVAIRSRENVVFVQTDKPTYKPGQKV

SEQ ID NO: 3 - DNA, 56 bp, Homo sapiens
ttcatatattaacattatttttatttttatttcagtatccagtgatcacccttcag

SEQ ID NO: 4 - protein, 18 aa, Homo sapiens
HILTLFLFLFQYPVITLQ

SEQ ID NO: 5 - DNA, 169 bp, Homo sapiens
gatcctcaaaacaatcggatttttcaaaggcaaaatgtgacttctttccgaaatattacccaactctcgttccaactgatttcagaaccaatgtttggagattactggattgttgtgaaaagaaactcaagggagacagtgacacaccaatttgctgttaaaagatatg

SEQ ID NO: 6 - protein, 57 aa, Homo sapiens
DPQNNRIFQRQNVTSFRNITQLSFQLISEPMFGDYWIVVKRNSRETVTHQFAVKRYV

SEQ ID NO: 7 - DNA, 85 bp, Homo sapiens
tgctgcccaagtttgaagttacagtcaatgcaccacaaacagtaactatttcagatgatgaattccaagtggatgtatgtgctaa

SEQ ID NO: 8 - protein, 28 aa, Homo sapiens
LPKFEVTVNAPQTVTISDDEFQVDVCAK

SEQ ID NO: 9 - DNA, 118 bp, Homo sapiens
gtacaactttggccaacctgtgcaaggggaaacccaaatccgggtgtgcagagagtatttttcttcaagcaattgtgagaaaaatgaaaatgaaatatgtgagcaatttattgcacag

SEQ ID NO: 10 - protein, 39 aa, Homo sapiens
YNFGQPVQGETQIRVCREYFSSSNCEKNENEICEQFIAQ

SEQ ID NO: 11 - DNA, 112 bp, Homo sapiens
ttggaaaatggttgtgtttctcaaattgtaaatacaaaagtcttccaactctaccgttcgggattgttcatgacatttcatgtcgctgtaattgttacagaatctgggacag

SEQ ID NO: 12 - protein, 38 aa, Homo sapiens
LENGCVSQIVNTKVFQLYRSGLFMTFHVAVIVTESGTV

SEQ ID NO: 13 - DNA, 110 bp, Homo sapiens
ttatgcagatcagcgagaagacctcagtttttatcactcaattgcttggaactgtaaactttgagaacatggatacattctatagaagagggatttcttattttggaact

SEQ ID NO: 14 - protein, 36 aa, Homo sapiens
MQISEKTSVFITQLLGTVNFENMDTFYRRGISYFGT

SEQ ID NO: 15 - DNA, 159 bp, Homo sapiens
cttaaattttcggatcccaataatgtacctatggtgaacaagttgttgcaactggagctcaatgatgaatttataggaaattacactacggatgagaatggcgaagctcaattttccattgacacttcagacatatttgatccagagttcaacctaaaa

SEQ ID NO: 16 - protein, 53 aa, Homo sapiens
LKFSDPNNVPMVNKLLQLELNDEFIGNYTTDENGEAQFSIDTSDIFDPEFNLK

SEQ ID NO: 17 - DNA, 228 bp, Homo sapiens
gccacatatgttcgacctgagagctgctatcttcccagctggttgacgcctcagtacttggatgctcacttcttagtctcacgcttttactcccgaaccaacagcttcctgaagattgttccagaaccaaagcagcttgaatgtaatcaacagaaggttgttactgtgcattactccctaaacagtgaagcatatgaggatgattccaatgtaaagttcttctatttg

SEQ ID NO: 18 - protein, 76 aa, Homo sapiens
ATYVRPESCYLPSWLTPQYLDAHFLVSRFYSRTNSFLKIVPEPKQLECNQQKVVTVHYSLNSEAYEDDSNVKFFYL

SEQ ID NO: 19 - DNA, 55 bp, Homo sapiens
atgatggtaaaaggagctatcttactcagtggacaaaaggaaatcagaaacaaag

SEQ ID NO: 20 - protein, 19 aa, Homo sapiens
MMVKGAILLSGQKEIRNKA

SEQ ID NO: 21 - DNA, 146 bp, Homo sapiens
cctggaatggaaacttctcgttcccaatcagcatcagtgctgatctggctcctgcagccgtcctgtttgtctatacccttcaccccagtggggaaattgtggctgacagtgtcagattccaggttgacaagtgctttaaacacaag

SEQ ID NO: 22 - protein, 48 aa, Homo sapiens
WNGNFSFPISISADLAPAAVLFVYTLHPSGEIVADSVRFQVDKCFKHK

SEQ ID NO: 23 - DNA, 150 bp, Homo sapiens
gttaacataaagttctctaacgagcagggcttacctggttccaatgctagtctctgtcttcaagcggcgcctgtcttattctgtgccctcagggctgtggataggaatgtccttctactgaaatctgaacaacagctgtcagctgaaagt

SEQ ID NO: 24 - protein, 50 aa, Homo sapiens
VNIKFSNEQGLPGSNASLCLQAAPVLFCALRAVDRNVLLLKSEQQLSAES

SEQ ID NO: 25 - DNA, 171 bp, Homo sapiens
gtgtataacatggttccaagtatagagccgtatggttatttctaccatggcctcaatcttgatgatggcaaggaagacccttgcattcctcagagggatatgttctacaatggtttatattacacacctgtaagcaactatggggatggagatatctataatattgtcagg

SEQ ID NO: 26 - protein, 57 aa, Homo sapiens
VYNMVPSIEPYGYFYHGLNLDDGKEDPCIPQRDMFYNGLYYTPVSNYGDGDIYNIVR

SEQ ID NO: 27 - DNA, 149 bp, Homo sapiens
aacatgggtctaaaagtctttaccaatctccattaccgaaaaccagaagtatgtgtgatggagagaaggctgccactccctaagccgctttatctggaaacagaaaattatggtccaatgcgtagtgttccgtctagaattgcatgtag

SEQ ID NO: 28 - protein, 50 aa, Homo sapiens
NMGLKVFTNLHYRKPEVCVMERRLPLPKPLYLETENYGPMRSVPSRIACR

SEQ ID NO: 29 - DNA, 93 bp, Homo sapiens
aggggagaatgctgactatgtagaacaggctataattcaaacagtaagaacaaacttcccagagacatggatgtgggacctcgtcagtgtcga

SEQ ID NO: 30 - protein, 31 aa, Homo sapiens
GENADYVEQAIIQTVRTNFPETWMWDLVSVD

SEQ ID NO: 31 - DNA, 229 bp, Homo sapiens
ttcctcaggctctgccaatctttcgttcctcattcctgatacgataacccaatgggaggcaagtggcttttgtgtgaatggtgacgttggatttggcatttcctctacaaccactctagaagtctcccaacctttctttattgagattgcctcacccttttcggttgttcaaaatgaacaatttgatttgattgtcaatgtcttcagctaccggaatacatgtgtagag

SEQ ID NO: 32 - protein, 76 aa, Homo sapiens
SSGSANLSFLIPDTITQWEASGFCVNGDVGFGISSTTTLEVSQPFFIEIASPFSVVQNEQFDLIVNVFSYRNTCVE

SEQ ID NO: 33 - DNA, 130 bp, Homo sapiens
atttctgttcaagtggaggagtctcagaattatgaagcaaatattcataccttgaaaatcaatggcagtgaggttattcaagctggagggaggaaaacaaacgtctggactattatacctaagaaattgg

SEQ ID NO: 34 - protein, 44 aa, Homo sapiens
ISVQVEESQNYEANIHTLKINGSEVIQAGGRKTNVWTIIPKKLG

SEQ ID NO: 35 - DNA, 119 bp, Homo sapiens
gtaaagtgaatatcactgtagttgctgagtccaaacaaagcagtgcttgcccaaatgaaggaatggagcagcaaaagctaaactggaaagacactgtggtccaaagcttcttagtagag

SEQ ID NO: 36 - protein, 39 aa, Homo sapiens
KVNITVVAESKQSSACPNEGMEQQKLNWKDTVVQSFLVE

SEQ ID NO: 37 - DNA, 52 bp, Homo sapiens
cctgaaggtattgaaaaggaaaggacccagagtttccttatctgtacagaag

SEQ ID NO: 38 - protein, 18 aa, Homo sapiens
PEGIEKERTQSFLICTEG

SEQ ID NO: 39 - DNA, 84 bp, Homo sapiens
gtgccaaagcctccaagcagggagttttggacttgccaaacgatgtagtagaagggtcagccagaggctttttcactgttgtgg

SEQ ID NO: 40 - protein, 28 aa, Homo sapiens
AKASKQGVLDLPNDVVEGSARGFFTVVG

SEQ ID NO: 41 - DNA, 174 bp, Homo sapiens
gggatattctaggacttgccttgcagaatctggttgttctccaaatgccctatggaagtggagagcagaatgctgccctactagcatctgatacttatgttctggactatctgaaatctactgagcaactgacagaggaagttcaatctaaggctttctttctcttatctaatg

SEQ ID NO: 42 - protein, 58 aa, Homo sapiens
DILGLALQNLVVLQMPYGSGEQNAALLASDTYVLDYLKSTEQLTEEVQSKAFFLLSNG

SEQ ID NO: 43 - DNA, 82 bp, Homo sapiens
gttatcaaaggcaattatctttcaaaaactctgatggttcctatagtgtgttttggcagcagagtcagaaaggaagcatatg

SEQ ID NO: 44 - protein, 27 aa, Homo sapiens
YQRQLSFKNSDGSYSVFWQQSQKGSIC

SEQ ID NO: 45 - DNA, 151 bp, Homo sapiens
tgctcttacttttaagacattggagagaatgaaaaaatatgtattcattgatgaaaatgttcaaaaacagaccttaatctggctttcaagccaacagaaaacaagcggctgctttaagaatgatggccagcttttcaaccacgcctgggag

SEQ ID NO: 46 - protein, 50 aa, Homo sapiens
ALTFKTLERMKKYVFIDENVQKQTLIWLSSQQKTSGCFKNDGQLFNHAWE

SEQ ID NO: 47 - DNA, 75 bp, Homo sapiens
ggtggagatgaagaggacatttcactcactgcgtatgttgttgggatgttctttgaagctgggctcaatttcact

SEQ ID NO: 48 - protein, 25 aa, Homo sapiens
GGDEEDISLTAYVVGMFFEAGLNFT

SEQ ID NO: 49 - DNA, 166 bp, Homo sapiens
tttcctgctctacgaaacgcactcttttgccttgaagcggcattggacagtggtgtcactaatggctataatcatgcaattctagcttatgcttttgccttagctggaaaagagaagcaagtggaatctttactccaaaccctggatcaatctgccccaaaactaa

SEQ ID NO: 50 - protein, 56 aa, Homo sapiens
FPALRNALFCLEAALDSGVTNGYNHAILAYAFALAGKEKQVESLLQTLDQSAPKLN

SEQ ID NO: 51 - DNA, 212 bp, Homo sapiens
ataatgtcatctactgggaaagagaaaggaaacccaagacagaagaatttccatcctttattccctgggcaccttctgctcagactgagaagagttgctacgtgctgttggctgtcatttcccggaaaattcctgacctcacctatgctagtaagattgtgcagtggcttgcccaacggatgaattcccatggaggcttttcttccaaccag

SEQ ID NO: 52 - protein, 70 aa, Homo sapiens
NVIYWERERKPKTEEFPSFIPWAPSAQTEKSCYVLLAVISRKIPDLTYASKIVQWLAQRMNSHGGFSSNQ

SEQ ID NO: 53 - DNA, 43 bp, Homo sapiens
gaaactgcagtttgtcttcttgccataacccgctacataaccc

SEQ ID NO: 54 - protein, 15 aa, Homo sapiens [sequence omitted]

SEQ ID NO: 55 - DNA, 170 bp, Homo sapiens
aggggctcttctctaaggatcaaaacactgtcacctttagcagtgaaggatccagtgagattttccaggttaacggtcataaccgcctactggtccaacgttcagaagtaacacaggcacctggagaatacacagtagatgtggaaggacacggttgtacatttatccag

SEQ ID NO: 56 - protein, 56 aa, Homo sapiens
GLFSKDQNTVTFSSEGSSEIFQVNGHNRLLVQRSEVTQAPGEYTVDVEGHGCTFIQ

SEQ ID NO: 57 - DNA, 113 bp, Homo sapiens
gccacccttaagtacaatgttctcctacctaagaaggcatctggattttctctttccttggaaatagtaaagaactactcttcgactgcttttgacctcacagtgaccctcaa

SEQ ID NO: 58 - protein, 38 aa, Homo sapiens
ATLKYNVLLPKKASGFSLSLEIVKNYSSTAFDLTVTLK

SEQ ID NO: 59 - DNA, 91 bp, Homo sapiens
atacactggaattcgcaataaatccagtatggtggttatagatgtaaaaatgctatcaggatttactccaaccatgtcatccattgaagag

SEQ ID NO: 60 - protein, 30 aa, Homo sapiens
YTGIRNKSSMVVIDVKMLSGFTPTMSSIEE

SEQ ID NO: 61 - DNA, 69 bp, Homo sapiens
cttgaaaacaagggccaagtgatgaagactgaagtcaagaatgaccatgttcttttctacttggaaaat

SEQ ID NO: 62 - protein, 23 aa, Homo sapiens
LENKGQVMKTEVKNDHVLFYLEN

SEQ ID NO: 63 - DNA, 103 bp, Homo sapiens
ggttttggtcgagcagacagtttccctttttctgttgagcagagcaaccttgtgttcaacattcagccagccccagccatggtctacgattattacgaaaaag

SEQ ID NO: 64 - protein, 35 aa, Homo sapiens
GFGRADSFPFSVEQSNLVFNIQPAPAMVYDYYEKE

SEQ ID NO: 65 - DNA, 53 bp, Homo sapiens
aagaatatgccctagctttttacaacatcgacagtagttcagtttcccagtga

SEQ ID NO: 66 - protein, 16 aa, Homo sapiens
EYALAFYNIDSSSVSQ

SEQ ID NO: 67 - DNA, 4074 bp, Homo sapiens
gttcctcaggccagatctgacccactggcatttattacattttctgctaaaggagccactctcaacctggaagagaggagatctgtggcaatcagatccagagagaatgtggtcttcgtacagactgataaacccacctacaagcctggacagaaagttcatatattaacattatttttatttttatttcagtatccagtgatcacccttcaggatcctcaaaacaatcggatttttcaaaggcaaaatgtgacttctttccgaaatattacccaactctcgttccaactgatttcagaaccaatgtttggagattactggattgttgtgaaaagaaactcaagggagacagtgacacaccaatttgctgttaaaagatatgtgctgcccaagtttgaagttacagtcaatgcaccacaaacagtaactatttcagatgatgaattccaagtggatgtatgtgctaagtacaactttggccaacctgtgcaaggggaaacccaaatccgggtgtgcagagagtatttttcttcaagcaattgtgagaaaaatgaaaatgaaatatgtgagcaatttattgcacagttggaaaatggttgtgtttctcaaattgtaaatacaaaagtcttccaactctaccgttcgggattgttcatgacatttcatgtcgctgtaattgttacagaatctgggacagttatgcagatcagcgagaagacctcagtttttatcactcaattgcttggaactgtaaactttgagaacatggatacattctatagaagagggatttcttattttggaactcttaaattttcggatcccaataatgtacctatggtgaacaagttgttgcaactggagctcaatgatgaatttataggaaattacactacggatgagaatggcgaagctcaattttccattgacacttcagacatatttgatccagagttcaacctaaaagccacatatgttcgacctgagagctgctatcttcccagctggttgacgcctcagtacttggatgctcacttcttagtctcacgcttttactcccgaaccaacagcttcctgaagattgttccagaaccaaagcagcttgaatgtaatcaacagaaggttgttactgtgcattactccctaaacagtgaagcatatgaggatgattccaatgtaaagttcttctatttgatgatggtaaaaggagctatcttactcagtggacaaaaggaaatcagaaacaaagcctggaatggaaacttctcgttcccaatcagcatcagtgctgatctggctcctgcagccgtcctgtttgtctatacccttcaccccagtggggaaattgtggctgacagtgtcagattccaggttgacaagtgctttaaacacaaggttaacataaagttctctaacgagcagggcttacctggttccaatgctagtctctgtcttcaagcggcgcctgtcttattctgtgccctcagggctgtggataggaatgtccttctactgaaatctgaacaacagctgtcagctgaaagtgtgtataacatggttccaagtatagagccgtatggttatttctaccatggcctcaatcttgatgatggcaaggaagacccttgcattcctcagagggatatgttctacaatggtttatattacacacctgtaagcaactatggggatggagatatctataatattgtcaggaacatgggtctaaaagtctttaccaatctccattaccgaaaaccagaagtatgtgtgatggagagaaggctgccactccctaagccgctttatctggaaacagaaaattatggtccaatgcgtagtgttccgtctagaattgcatgtagaggggagaatgctgactatgtagaacaggctataattcaaacagtaagaacaaacttcccagagacatggatgtgggacctcgtcagtgtcgattcctcaggctctgccaatctttcgttcctcattcctgatacgataacccaatgggaggcaagtggcttttgtgtgaatggtgacgttggatttggcatttcctctacaaccactctagaagtctcccaacctttctttattgagattgcctcacccttttcggttgttcaaaatgaacaatttgatttgattgtcaatgtcttcagctaccggaatacatgtgtagagatttctgttcaagtggaggagtctcagaattatgaagcaaatattcataccttgaaaatcaatggcagtgaggttattcaagctggagggaggaaaacaaacgtctggactattatacctaagaaattgggtaaagtgaatatcactgtagttgctgagtccaaacaaagcagtgcttgcccaaatgaaggaatggagcagcaaaagctaaactggaaagacactgtggtccaaagcttcttagtagagcctgaaggtattgaaaaggaaaggacccagagtttccttatctgtacagaaggtgccaaagcctccaagcagggagttttggacttgccaaacgatgtagtagaagggtcagccagaggctttttcactgttgtgggggatattctaggacttgccttgcagaatctggttgttctccaaatgccctatggaagtggagagcagaatgctgccctactagcatctgatacttatgttctggactatctgaaatctactgagcaactgacagaggaagttcaatctaaggctttctttctcttatctaatggttatcaaaggcaattatctttcaaaaactctgatggttcctatagtgtgttttggcagcagagtcagaaaggaagcatatgtgctcttacttttaagacattggagagaatgaaaaaatatgtattcattgatgaaaatgttcaaaaacagaccttaatctggctttcaagccaacagaaaacaagcggctgctttaagaatgatggccagcttttcaaccacgcctgggagggtggagatgaagaggacatttcactcactgcgtatgttgttgggatgttctttgaagctgggctcaatttcacttttcctgctctacgaaacgcactcttttgccttgaagcggcattggacagtggtgtcactaatggctataatcatgcaattctagcttatgcttttgccttagctggaaaagagaagcaagtggaatctttactccaaaccctggatcaatctgccccaaaactaaataatgtcatctactgggaaagagaaaggaaacccaagacagaagaatttccatcctttattccctgggcaccttctgctcagactgagaagagttgctacgtgctgttggctgtcatttcccggaaaattcctgacctcacctatgctagtaagattgtgcagtggcttgcccaacggatgaattcccatggaggcttttcttccaaccaggaaactgcagtttgtcttcttgccataacccgctacataacccaggggctcttctctaaggatcaaaacactgtcacctttagcagtgaaggatccagtgagattttccaggttaacggtcataaccgcctactggtccaacgttcagaagtaacacaggcacctggagaatacacagtagatgtggaaggacacggttgtacatttatccaggccacccttaagtacaatgttctcctacctaagaaggcatctggattttctctttccttggaaatagtaaagaactactcttcgactgcttttgacctcacagtgaccctcaaatacactggaattcgcaataaatccagtatggtggttatagatgtaaaaatgctatcaggatttactccaaccatgtcatccattgaagagcttgaaaacaagggccaagtgatgaagactgaagtcaagaatgaccatgttcttttctacttggaaaatggttttggtcgagcagacagtttccctttttctgttgagcagagcaaccttgtgttcaacattcagccagccccagccatggtctacgattattacgaaaaagaagaatatgccctagctttttacaacatcgacagtagttcagtttcccagtga

SEQ ID NO: 68 - protein, 1357 aa, Homo sapiens
VPQARSDPLAFITFSAKGATLNLEERRSVAIRSRENVVFVQTDKPTYKPGQKVHILTLFLFLFQYPVITLQDPQNNRIFQRQNVTSFRNITQLSFQLISEPMFGDYWIVVKRNSRETVTHQFAVKRYVLPKFEVTVNAPQTVTISDDEFQVDVCAKYNFGQPVQGETQIRVCREYFSSSNCEKNENEICEQFIAQLENGCVSQIVNTKVFQLYRSGLFMTFHVAVIVTESGTVMQISEKTSVFITQLLGTVNFENMDTFYRRGISYFGTLKFSDPNNVPMVNKLLQLELNDEFIGNYTTDENGEAQFSIDTSDIFDPEFNLKATYVRPESCYLPSWLTPQYLDAHFLVSRFYSRTNSFLKIVPEPKQLECNQQKVVTVHYSLNSEAYEDDSNVKFFYLMMVKGAILLSGQKEIRNKAWNGNFSFPISISADLAPAAVLFVYTLHPSGEIVADSVRFQVDKCFKHKVNIKFSNEQGLPGSNASLCLQAAPVLFCALRAVDRNVLLLKSEQQLSAESVYNMVPSIEPYGYFYHGLNLDDGKEDPCIPQRDMFYNGLYYTPVSNYGDGDIYNIVRNMGLKVFTNLHYRKPEVCVMERRLPLPKPLYLETENYGPMRSVPSRIACRGENADYVEQAIIQTVRTNFPETWMWDLVSVDSSGSANLSFLIPDTITQWEASGFCVNGDVGFGISSTTTLEVSQPFFIEIASPFSVVQNEQFDLIVNVFSYRNTCVEISVQVEESQNYEANIHTLKINGSEVIQAGGRKTNVWTIIPKKLGKVNITVVAESKQSSACPNEGMEQQKLNWKDTVVQSFLVEPEGIEKERTQSFLICTEGAKASKQGVLDLPNDVVEGSARGFFTVVGDILGLALQNLVVLQMPYGSGEQNAALLASDTYVLDYLKSTEQLTEEVQSKAFFLLSNGYQRQLSFKNSDGSYSVFWQQSQKGSICALTFKTLERMKKYVFIDENVQKQTLIWLSSQQKTSGCFKNDGQLFNHAWEGGDEEDISLTAYVVGMFFEAGLNFTFPALRNALFCLEAALDSGVTNGYNHAILAYAFALAGKEKQVESLLQTLDQSAPKLNNVIYWERERKPKTEEFPSFIPWAPSAQTEKSCYVLLAVISRKIPDLTYASKIVQWLAQRMNSHGGFSSNQETAVCLLAITRYITQGLFSKDQNTVTFSSEGSSEIFQVNGHNRLLVQRSEVTQAPGEYTVDVEGHGCTFIQATLKYNVLLPKKASGFSLSLEIVKNYSSTAFDLTVTLKYTGIRNKSSMVVIDVKMLSGFTPTMSSIEELENKGQVMKTEVKNDHVLFYLENGFGRADSFPFSVEQSNLVFNIQPAPAMVYDYYEKEEYALAFYNIDSSSVSQ

SEQ ID NO: 69 - DNA, 153 bp, Homo sapiens
caggtttcccttggcttctccccctcccagcagcttccaggagcagaagtggagctgcagctgcaggcagctcccggatccctgtgtgcgctccgggcggtggatgagagtgtcttactgcttaggccagacagagagctgagcaaccgctct

SEQ ID NO: 70 - protein, 51 aa, Homo sapiens
QVSLGFSPSQQLPGAEVELQLQAAPGSLCALRAVDESVLLLRPDRELSNRS

SEQ ID NO: 71 - DNA, 195 bp, Homo sapiens
gtctatgggatgtttccattctggtatggtcactacccctatcaagtggctgagtatgatcagtgtccagtgtctggcccatgggactttcctcagcccctcattgacccaatgccccaagggcattcgagccagcgttccattatctggaggccctcgttctctgaaggcacggaccttttcagctttttccgg

SEQ ID NO: 72 - protein, 65 aa, Homo sapiens
VYGMFPFWYGHYPYQVAEYDQCPVSGPWDFPQPLIDPMPQGHSSQRSIIWRPSFSEGTDLFSFFR

SEQ ID NO: 73 - DNA, 91 bp, Homo sapiens
gacgtgggcctgaaaatactgtccaatgccaaaatcaagaagccagtagattgcagtcacagatctccagaatacagcactgctatgggtg

SEQ ID NO: 74 - protein, 31 aa, Homo sapiens
DVGLKILSNAKIKKPVDCSHRSPEYSTAMGA

SEQ ID NO: 75 - DNA, 115 bp, Homo sapiens
caggcggtggtcatccagaggcttttgagtcatcaactcctttacatcaagcagaggattctcaggtccgccagtacttcccagagacctggctctgggatctgtttcctattgg

SEQ ID NO: 76 - protein, 38 aa, Homo sapiens
GGGHPEAFESSTPLHQAEDSQVRQYFPETWLWDLFPIG

SEQ ID NO: 77 - DNA, 229 bp, Homo sapiens
taactcggggaaggaggcggtccacgtcacagttcctgacgccatcaccgagtggaaggcgatgagtttctgcacttcccagtcaagaggcttcgggctttcacccactgttggactaactgctttcaagccgttctttgttgacctgactctcccttactcagtagtccgtggggaatcctttcgtcttactgccaccatcttcaattacctaaaggattgcatcagg

SEQ ID NO: 78 - protein, 76 aa, Homo sapiens
NSGKEAVHVTVPDAITEWKAMSFCTSQSRGFGLSPTVGLTAFKPFFVDLTLPYSVVRGESFRLTATIFNYLKDCIR

SEQ ID NO: 79 - DNA, 127 bp, Homo sapiens
gttcagactgacctggctaaatcgcatgagtaccagctagaatcatgggcagattctcagacctccagttgtctctgtgctgatgacgcaaaaacccaccactggaacatcacagctgtcaaattgg

SEQ ID NO: 80 - protein, 43 aa, Homo sapiens
VQTDLAKSHEYQLESWADSQTSSCLCADDAKTHHWNITAVKLG

SEQ ID NO: 81 - DNA, 122 bp, Homo sapiens
gtcacattaactttactattagtacaaagattctggacagcaatgaaccatgtgggggccagaaggggtttgttccccaaaagggccgaagtgacacgctcatcaagccagttctcgtcaaa

SEQ ID NO: 82 - protein, 40 aa, Homo sapiens
HINFTISTKILDSNEPCGGQKGFVPQKGRSDTLIKPVLVK

SEQ ID NO: 83 - DNA, 52 bp, Homo sapiens
cctgagggagtcctggtggagaagacacacagctcattgctgtgcccaaaag

SEQ ID NO: 84 - protein, 18 aa, Homo sapiens
PEGVLVEKTHSSLLCPKG

SEQ ID NO: 85 - DNA, 84 bp, Homo sapiens
gaaaggtggcatctgaatctgtctccctggagctcccagtggacattgttcctgactcgaccaaggcttatgttacggttctgg

SEQ ID NO: 86 - protein, 28 aa, Homo sapiens
KVASESVSLELPVDIVPDSTKAYVTVLG

SEQ ID NO: 87 - DNA, 177 bp, Homo sapiens
gagacattatgggcacagccctgcagaacctggatggtctggtgcagatgcccagtggctgtggcgagcagaacatggtcttgtttgctcccatcatctatgtcttgcagtacctggagaaggcagggctgctgacggaggagatcaggtctcgggcagtgggtttcctggaaatag

SEQ ID NO: 88 - protein, 59 aa, Homo sapiens
DIMGTALQNLDGLVQMPSGCGEQNMVLFAPIIYVLQYLEKAGLLTEEIRSRAVGFLEIG

SEQ ID NO: 89 - DNA, 82 bp, Homo sapiens
ggtaccagaaggagctgatgtacaaacacagcaatggctcatacagtgcctttggggagcgagatggaaatggaaacacatg

SEQ ID NO: 90 - protein, 27 aa, Homo sapiens
YQKELMYKHSNGSYSAFGERDGNGNTW

SEQ ID NO: 91 - DNA, 157 bp, Homo sapiens
gctgacagcgtttgtcacaaaatgctttggccaagctcagaaattcatcttcattgatcccaagaacatccaggatgctctcaagtggatggcaggaaaccagctccccagtggctgctatgccaacgtgggaaatctccttcacacagctatgaag

SEQ ID NO: 92 - protein, 52 aa, Homo sapiens
LTAFVTKCFGQAQKFIFIDPKNIQDALKWMAGNQLPSGCYANVGNLLHTAMK

SEQ ID NO: 93 - DNA, 75 bp, Homo sapiens
ggtggtgttgatgatgaggtctccttgactgcgtatgtcacagctgcattgctggagatgggaaaggatgtagat

SEQ ID NO: 94 - protein, 25 aa, Homo sapiens
GGVDDEVSLTAYVTAALLEMGKDVD

SEQ ID NO: 95 - DNA, 163 bp, Homo sapiens
gacccaatggtgagtcagggtctacggtgtctcaagaattcggccacctccacgaccaacctctacacacaggccctgttggcttacattttctccctggctggggaaatggacatcagaaacattctccttaaacagttagatcaacaggctatcatctcag

SEQ ID NO: 96 - protein, 55 aa, Homo sapiens
DPMVSQGLRCLKNSATSTTNLYTQALLAYIFSLAGEMDIRNILLKQLDQQAIISG

SEQ ID NO: 97 - DNA, 215 bp, Homo sapiens
gagaatccatttactggagccagaaacctactccatcatcgaacgccagcccttggtctgagcctgcggctgtagatgtggaactcacagcatatgcattgttggcccagcttaccaagcccagcctgactcaaaaggagatagcgaaggccactagcatagtggcttggttggccaagcaacacaatgcatatgggggcttctcttctactcag

SEQ ID NO: 98 - protein, 71 aa, Homo sapiens
ESIYWSQKPTPSSNASPWSEPAAVDVELTAYALLAQLTKPSLTQKEIAKATSIVAWLAKQHNAYGGFSSTQ

SEQ ID NO: 99 - DNA, 216 bp, Homo sapiens
gatactgtagttgctctccaagctcttgccaaatatgccactaccgcctacatgccatctgaggagatcaacctggttgtaaaatccactgagaatttccagcgcacattcaacatacagtcagttaacagattggtatttcagcaggataccctgcccaatgtccctggaatgtacacgttggaggcctcaggccagggctgtgtctatgtgcag

SEQ ID NO: 100 - protein, 72 aa, Homo sapiens
DTVVALQALAKYATTAYMPSEEINLVVKSTENFQRTFNIQSVNRLVFQQDTLPNVPGMYTLEASGQGCVYVQ

SEQ ID NO: 101 - DNA, 128 bp, Homo sapiens
acggtgttgagatacaatattctccctcccacaaatatgaagacctttagtcttagtgtggaaataggaaaagctagatgtgagcaaccgacttcacctcgatccttgactctcactattcacaccag

SEQ ID NO: 102 - protein, 43 aa, Homo sapiens
TVLRYNILPPTNMKTFSLSVEIGKARCEQPTSPRSLTLTIHTS

SEQ ID NO: 103 - DNA, 91 bp, Homo sapiens
ttatgtggggagccgtagctcttccaatatggctattgtggaagtgaagatgctatctgggttcagtcccatggagggcaccaatcagtta

SEQ ID NO: 104 - protein, 30 aa, Homo sapiens
YVGSRSSSNMAIVEVKMLSGFSPMEGTNQL

SEQ ID NO: 105 - DNA, 69 bp, Homo sapiens
cttctccagcaacccctggtgaagaaggttgaatttggaactgacacacttaacatttacttggatgag

SEQ ID NO: 106 - protein, 23 aa, Homo sapiens
LLQQPLVKKVEFGTDTLNIYLDE

SEQ ID NO: 107 - DNA, 103 bp, Homo sapiens
ctcattaagaacactcagacttacaccttcaccatcagccaaagtgtgctggtcaccaacttgaaaccagcaaccatcaaggtctatgactactacctaccag

SEQ ID NO: 108 - protein, 35 aa, Homo sapiens
LIKNTQTYTFTISQSVLVTNLKPATIKVYDYYLPD

SEQ ID NO: 109 - DNA, 41 bp, Homo sapiens
atgaacaggcaacaattcagtattctgatccctgtgaatga

SEQ ID NO: 110 - protein, 12 aa, Homo sapiens
EQATIQYSDPCE

SEQ ID NO: 111 - DNA, 2685 bp, Homo sapiens
caggtttcccttggcttctccccctcccagcagcttccaggagcagaagtggagctgcagctgcaggcagctcccggatccctgtgtgcgctccgggcggtggatgagagtgtcttactgcttaggccagacagagagctgagcaaccgctctgtctatgggatgtttccattctggtatggtcactacccctatcaagtggctgagtatgatcagtgtccagtgtctggcccatgggactttcctcagcccctcattgacccaatgccccaagggcattcgagccagcgttccattatctggaggccctcgttctctgaaggcacggaccttttcagctttttccgggacgtgggcctgaaaatactgtccaatgccaaaatcaagaagccagtagattgcagtcacagatctccagaatacagcactgctatgggtgcaggcggtggtcatccagaggcttttgagtcatcaactcctttacatcaagcagaggattctcaggtccgccagtacttcccagagacctggctctgggatctgtttcctattggtaactcggggaaggaggcggtccacgtcacagttcctgacgccatcaccgagtggaaggcgatgagtttctgcacttcccagtcaagaggcttcgggctttcacccactgttggactaactgctttcaagccgttctttgttgacctgactctcccttactcagtagtccgtggggaatcctttcgtcttactgccaccatcttcaattacctaaaggattgcatcagggttcagactgacctggctaaatcgcatgagtaccagctagaatcatgggcagattctcagacctccagttgtctctgtgctgatgacgcaaaaacccaccactggaacatcacagctgtcaaattgggtcacattaactttactattagtacaaagattctggacagcaatgaaccatgtgggggccagaaggggtttgttccccaaaagggccgaagtgacacgctcatcaagccagttctcgtcaaacctgagggagtcctggtggagaagacacacagctcattgctgtgcccaaaaggaaaggtggcatctgaatctgtctccctggagctcccagtggacattgttcctgactcgaccaaggcttatgttacggttctgggagacattatgggcacagccctgcagaacctggatggtctggtgcagatgcccagtggctgtggcgagcagaacatggtcttgtttgctcccatcatctatgtcttgcagtacctggagaaggcagggctgctgacggaggagatcaggtctcgggcagtgggtttcctggaaatagggtaccagaaggagctgatgtacaaacacagcaatggctcatacagtgcctttggggagcgagatggaaatggaaacacatggctgacagcgtttgtcacaaaatgctttggccaagctcagaaattcatcttcattgatcccaagaacatccaggatgctctcaagtggatggcaggaaaccagctccccagtggctgctatgccaacgtgggaaatctccttcacacagctatgaagggtggtgttgatgatgaggtctccttgactgcgtatgtcacagctgcattgctggagatgggaaaggatgtagatgacccaatggtgagtcagggtctacggtgtctcaagaattcggccacctccacgaccaacctctacacacaggccctgttggcttacattttctccctggctggggaaatggacatcagaaacattctccttaaacagttagatcaacaggctatcatctcaggagaatccatttactggagccagaaacctactccatcatcgaacgccagcccttggtctgagcctgcggctgtagatgtggaactcacagcatatgcattgttggcccagcttaccaagcccagcctgactcaaaaggagatagcgaaggccactagcatagtggcttggttggccaagcaacacaatgcatatgggggcttctcttctactcaggatactgtagttgctctccaagctcttgccaaatatgccactaccgcctacatgccatctgaggagatcaacctggttgtaaaatccactgagaatttccagcgcacattcaacatacagtcagttaacagattggtatttcagcaggataccctgcccaatgtccctggaatgtacacgttggaggcctcaggccagggctgtgtctatgtgcagacggtgttgagatacaatattctccctcccacaaatatgaagacctttagtcttagtgtggaaataggaaaagctagatgtgagcaaccgacttcacctcgatccttgactctcactattcacaccagttatgtggggagccgtagctcttccaatatggctattgtggaagtgaagatgctatctgggttcagtcccatggagggcaccaatcagttacttctccagcaacccctggtgaagaaggttgaatttggaactgacacacttaacatttacttggatgagctcattaagaacactcagacttacaccttcaccatcagccaaagtgtgctggtcaccaacttgaaaccagcaaccatcaaggtctatgactactacctaccagatgaacaggcaacaattcagtattctgatccctgtgaatga

SEQ ID NO: 112 - protein, 894 aa, Homo sapiens
QVSLGFSPSQQLPGAEVELQLQAAPGSLCALRAVDESVLLLRPDRELSNRSVYGMFPFWYGHYPYQVAEYDQCPVSGPWDFPQPLIDPMPQGHSSQRSIIWRPSFSEGTDLFSFFRDVGLKILSNAKIKKPVDCSHRSPEYSTAMGAGGGHPEAFESSTPLHQAEDSQVRQYFPETWLWDLFPIGNSGKEAVHVTVPDAITEWKAMSFCTSQSRGFGLSPTVGLTAFKPFFVDLTLPYSVVRGESFRLTATIFNYLKDCIRVQTDLAKSHEYQLESWADSQTSSCLCADDAKTHHWNITAVKLGHINFTISTKILDSNEPCGGQKGFVPQKGRSDTLIKPVLVKPEGVLVEKTHSSLLCPKGKVASESVSLELPVDIVPDSTKAYVTVLGDIMGTALQNLDGLVQMPSGCGEQNMVLFAPIIYVLQYLEKAGLLTEEIRSRAVGFLEIGYQKELMYKHSNGSYSAFGERDGNGNTWLTAFVTKCFGQAQKFIFIDPKNIQDALKWMAGNQLPSGCYANVGNLLHTAMKGGVDDEVSLTAYVTAALLEMGKDVDDPMVSQGLRCLKNSATSTTNLYTQALLAYIFSLAGEMDIRNILLKQLDQQAIISGESIYWSQKPTPSSNASPWSEPAAVDVELTAYALLAQLTKPSLTQKEIAKATSIVAWLAKQHNAYGGFSSTQDTVVALQALAKYATTAYMPSEEINLVVKSTENFQRTFNIQSVNRLVFQQDTLPNVPGMYTLEASGQGCVYVQTVLRYNILPPTNMKTFSLSVEIGKARCEQPTSPRSLTLTIHTSYVGSRSSSNMAIVEVKMLSGFSPMEGTNQLLLQQPLVKKVEFGTDTLNIYLDELIKNTQTYTFTISQSVLVTNLKPATIKVYDYYLPDEQATIQYSDPCE

SEQ ID NO: 113 - protein, 95 aa, Homo sapiens
MVVIDVKMLSGFTPTMSSIEELENKGQVMKTEVKNDHVLFYLENGFGRADSFPFSVEQSNLVFNIQPAPAMVYDYYEKEEYALAFYNIDSSSVSQ

SEQ ID NO: 114 - DNA, 285 bp, Homo sapiens
atggtggttatagatgtaaaaatgctatcaggatttactccaaccatgtcatccattgaagagcttgaaaacaagggccaagtgatgaagactgaagtcaagaatgaccatgttcttttctacttggaaaatggttttggtcgagcagacagtttccctttttctgttgagcagagcaaccttgtgttcaacattcagccagccccagccatggtctacgattattacgaaaaagaagaatatgccctagctttttacaacatcgacagtagttcagtttcccag

SEQ ID NO: 115 - protein, 95 aa, Homo sapiens
MVVIDVKMLSGFTPTMSSIEELENKGQVMKTEVKNDHVLFYLENGFGRADSFPFSVEQSNLVFNIQPAPAMVYDYYEKEEYALAFYNIDSSSVSE

SEQ ID NO: 116 - DNA, 285 bp, Homo sapiens
atggtggttatagatgtaaaaatgctatcaggatttactccaaccatgtcatccattgaagagcttgaaaacaagggccaagtgatgaagactgaagtcaagaatgaccatgttcttttctacttggaaaatggttttggtcgagcagacagtttccctttttctgttgagcagagcaaccttgtgttcaacattcagccagccccagccatggtctacgattactatgaaaaagaagaatatgccctagctttttacaacatcgacagtagttcagtttccgag

SEQ ID NO: 117 - protein, 92 aa, Homo sapiens
NMAIVEVKMLSGFSPMEGTNQLLLQQPLVKKVEFGTDTLNIYLDELIKNTQTYTFTISQSVLVTNLKPATIKVYDYYLPDEQATIQYSDPCE

SEQ ID NO: 118 - DNA, 276 bp, Homo sapiens
aatatggctattgtggaagtgaagatgctatctgggttcagtcccatggagggcaccaatcagttacttctccagcaacccctggtgaagaaggttgaatttggaactgacacacttaacatttacttggatgagctcattaagaacactcagacttacaccttcaccatcagccaaagtgtgctggtcaccaacttgaaaccagcaaccatcaaggtctatgactactacctaccagatgaacaggcaacaattcagtattctgatccctgtgaa

SEQ ID NO: 119 - protein, 732 aa, Homo sapiens
QAEDSQVRQYFPETWLWDLFPIGNSGKEAVHVTVPDAITEWKAMSFCTSQSRGFGLSPTVGLTAFKPFFVDLTLPYSVVRGESFRLTATIFNYLKDCIRVQTDLAKSHEYQLESWADSQTSSCLCADDAKTHHWNITAVKLGHINFTISTKILDSNEPCGGQKGFVPQKGRSDTLIKPVLVKPEGVLVEKTHSSLLCPKGKVASESVSLELPVDIVPDSTKAYVTVLGDIMGTALQNLDGLVQMPSGCGEQNMVLFAPIIYVLQYLEKAGLLTEEIRSRAVGFLEIGYQKELMYKHSNGSYSAFGERDGNGNTWLTAFVTKCFGQAQKFIFIDPKNIQDALKWMAGNQLPSGCYANVGNLLHTAMKGGVDDEVSLTAYVTAALLEMGKDVDDPMVSQGLRCLKNSATSTTNLYTQALLAYIFSLAGEMDIRNILLKQLDQQAIISGESIYWSQKPTPSSNASPWSEPAAVDVELTAYALLAQLTKPSLTQKEIAKATSIVAWLAKQHNAYGGFSSTQDTVVALQALAKYATTAYMPSEEINLVVKSTENFQRTFNIQSVNRLVFQQDTLPNVPGMYTLEASGQGCVYVQTVLRYNILPPTNMKTFSLSVEIGKARCEQPTSPRSLTLTIHTSYVGSRSSSNMAIVEVKMLSGFSPMEGTNQLLLQQPLVKKVEFGTDTLNIYLDELIKNTQTYTFTISQSVLVTNLKPATIKVYDYYLPDEQATIQYSDPCE

SEQ ID NO: 120 - DNA, 2196 bp, Homo sapiens
caagcagaggattctcaggtccgccagtacttcccagagacctggctctgggatctgtttcctattggtaactcggggaaggaggcggtccacgtcacagttcctgacgccatcaccgagtggaaggcgatgagtttctgcacttcccagtcaagaggcttcgggctttcacccactgttggactaactgctttcaagccgttctttgttgacctgactctcccttactcagtagtccgtggggaatcctttcgtcttactgccaccatcttcaattacctaaaggattgcatcagggttcagactgacctggctaaatcgcatgagtaccagctagaatcatgggcagattctcagacctccagttgtctctgtgctgatgacgcaaaaacccaccactggaacatcacagctgtcaaattgggtcacattaactttactattagtacaaagattctggacagcaatgaaccatgtgggggccagaaggggtttgttccccaaaagggccgaagtgacacgctcatcaagccagttctcgtcaaacctgagggagtcctggtggagaagacacacagctcattgctgtgcccaaaaggaaaggtggcatctgaatctgtctccctggagctcccagtggacattgttcctgactcgaccaaggcttatgttacggttctgggagacattatgggcacagccctgcagaacctggatggtctggtgcagatgcccagtggctgtggcgagcagaacatggtcttgtttgctcccatcatctatgtcttgcagtacctggagaaggcagggctgctgacggaggagatcaggtctcgggcagtgggtttcctggaaatagggtaccagaaggagctgatgtacaaacacagcaatggctcatacagtgcctttggggagcgagatggaaatggaaacacatggctgacagcgtttgtcacaaaatgctttggccaagctcagaaattcatcttcattgatcccaagaacatccaggatgctctcaagtggatggcaggaaaccagctccccagtggctgctatgccaacgtgggaaatctccttcacacagctatgaagggtggtgttgatgatgaggtctccttgactgcgtatgtcacagctgcattgctggagatgggaaaggatgtagatgacccaatggtgagtcagggtctacggtgtctcaagaattcggccacctccacgaccaacctctacacacaggccctgttggcttacattttctccctggctggggaaatggacatcagaaacattctccttaaacagttagatcaacaggctatcatctcaggagaatccatttactggagccagaaacctactccatcatcgaacgccagcccttggtctgagcctgcggctgtagatgtggaactcacagcatatgcattgttggcccagcttaccaagcccagcctgactcaaaaggagatagcgaaggccactagcatagtggcttggttggccaagcaacacaatgcatatgggggcttctcttctactcaggatactgtagttgctctccaagctcttgccaaatatgccactaccgcctacatgccatctgaggagatcaacctggttgtaaaatccactgagaatttccagcgcacattcaacatacagtcagttaacagattggtatttcagcaggataccctgcccaatgtccctggaatgtacacgttggaggcctcaggccagggctgtgtctatgtgcagacggtgttgagatacaatattctccctcccacaaatatgaagacctttagtcttagtgtggaaataggaaaagctagatgtgagcaaccgacttcacctcgatccttgactctcactattcacaccagttatgtggggagccgtagctcttccaatatggctattgtggaagtgaagatgctatctgggttcagtcccatggagggcaccaatcagttacttctccagcaacccctggtgaagaaggttgaatttggaactgacacacttaacatttacttggatgagctcattaagaacactcagacttacaccttcaccatcagccaaagtgtgctggtcaccaacttgaaaccagcaaccatcaaggtctatgactactacctaccagatgaacaggcaacaattcagtattctgatccctgtgaa

SEQ ID NO: 121 - protein, 745 aa, Homo sapiens
GHPEAFESSTPLHQAEDSQVRQYFPETWLWDLFPIGNSGKEAVHVTVPDAITEWKAMSFCTSQSRGFGLSPTVGLTAFKPFFVDLTLPYSVVRGESFRLTATIFNYLKDCIRVQTDLAKSHEYQLESWADSQTSSCLCADEAKTHHWNITAVKLGHINFTISTKILDSNEPCGGQKGFVPQKGRSDTLIKPVLVKPEGVLVEKTHSSLLCPKGKVASESVSLELPVDIVPDSTKAYVTVLGDIMGTALQNLDGLVQMPSGCGEQNMVLFAPIIYVLQYLEKAGLLTEEIRSRAVGFLEIGYQKELMYKHSNGSYSAFGERDGNGNTWLTAFVTKCFGQAQKFIFIDPKNIQDALKWMAGNQLPSGCYANVGNLLHTAMKGGVDDEVSLTAYVTAALLEMGKDVDDPMVSQGLWCLKNSATSTTNLYTQALLAYIFSLAGEMDIRNILLKQLDQQAIISGESIYWSQKPTPSSNASPWSEPAAVDVELTAYALLAQLTKPSLTQKEIAKATSIVAWLAKQRNAYGGFSSTQDTVVALQALAKYATTAYVPSEEINLVVKSTENFQRTFNIQSVNRLVFQQDTLPNVPGMYTLEASGQGCVYVQTVLRYNILPPTNMKTFSLSVEIGKARCEQPTSPRSLTLTIHTSYVGSRSSSNMAIVEVKMLSGFSPMEGTNQLLLQQPLVKKVEFGTDTLNIYLDELIKNTQTYTFTISQSVLVTNLKPATIKVYDYYLPDEQATIQYSDPCE

SEQ ID NO: 122 - DNA, 2237 bp, Homo sapiens
gtggtcatccagaggcttttgagtcatcaactcctttacatcaagcagaggattctcaggtccgccagtacttcccagagacctggctctgggatctgtttcctattggtaactcggggaaggaggcggtccacgtcacagttcctgacgccatcaccgagtggaaggcgatgagtttctgcacttcccagtcaagaggcttcgggctttcacccactgttggactaactgctttcaagccattctttgttgacctgactctcccttactcagtagtccgtggggaatcctttcgtcttactgccaccatcttcaattacctaaaggattgcatcagggttcagactgacctggctaaatcgcatgagtaccagctagaatcatgggcagattctcagacctccagttgtctctgtgctgatgaagcaaaaacccaccactggaacatcacagctgtcaaattgggtcacattaactttactattagtacaaagattctggacagcaatgaaccatgtgggggccagaaggggtttgttccccaaaagggccgaagtgacacgctcatcaagccagttctcgtcaaacctgagggagtcctggtggagaagacacacagctcattgctgtgcccaaaaggaaaggtggcatctgaatctgtctccctggagctcccagtggacattgttcctgactcgaccaaggcttatgttacggttctgggagacattatgggcacagccctgcagaacctggatggtctggtgcagatgcccagtggctgtggcgagcagaacatggtcttgtttgctcccatcatctatgtcttgcagtacctggagaaggcagggctgctgacggaggagatcaggtctcgggcagtgggtttcctggaaatagggtaccagaaggagctgatgtacaaacacagcaatggctcatacagtgcctttggggagcgagatggaaatggaaacacatggctgacagcgtttgtcacaaaatgctttggccaagctcagaaattcatcttcattgatcccaagaacatccaggatgctctcaagtggatggcaggaaaccagctccccagtggctgctatgccaacgtgggaaatctccttcacacagctatgaagggtggtgttgatgatgaggtctccttgactgcgtatgtcacagctgcattgctggagatgggaaaggatgtagatgacccaatggtgagtcagggtctatggtgtctcaagaattcggccacctccacgaccaacctctacacacaggccctgttggcttacattttctccctggctggggaaatggacatcagaaacattctccttaaacagttagatcaacaggctatcatctcaggagaatccatttactggagccagaaacctactccatcatcgaacgccagcccttggtctgagcctgcggctgtagatgtggaactcacagcatatgcattgttggcccagcttaccaagcccagcctgactcaaaaggagatagcgaaggccactagcatagtggcttggttggccaagcaacgcaatgcatatgggggcttctcttctactcaggatactgtagttgctctccaagctcttgccaaatatgccactaccgcctacgtgccatctgaggagatcaacctggttgtaaaatccactgagaatttccagcgcacattcaacatacagtcagttaacagattggtatttcagcaggataccctgcccaatgtccctggaatgtacacgttggaggcctcaggccagggctgtgtctatgtgcagacggtgttgagatacaatattctccctcccacaaatatgaagacctttagtcttagtgtggaaataggaaaagctagatgtgagcaaccgacttcacctcgatccttgactctcactattcacaccagttatgtggggagccgtagctcttccaatatggctattgtggaagtgaagatgctatctgggttcagtcccatggagggcaccaatcagttacttctccagcaacccctggtgaagaaggttgaatttggaactgacacacttaacatttacttggatgagctcattaagaacactcagacttacaccttcaccatcagccaaagtgtgctggtcaccaacttgaaaccagcaaccatcaaggtctatgactactacctaccagatgaacaggcaacaattcagtattctgatccctgtgaa

SEQ ID NO: 123 - DNA, 56 bp, Artificial Sequence
  INSP088-PCR1F Primer
ttttccctggtttttctggcatctcccctcgtggccattgtggaagtgaagatgct

SEQ ID NO: 124 - DNA, 35 bp, Artificial Sequence
  INSP088-PCR1R Primer
gtgatggtgatggtgacagggatcagaatactgaa

SEQ ID NO: 125 - DNA, 69 bp, Artificial Sequence
  IL12P40-SP1 Primer
atgtgtcaccagcagttggtcatctcttggttttccctggtttttctggcatctcccctcgtggccata

SEQ ID NO: 126 - DNA, 55 bp, Artificial Sequence
  GCPF-SP Primer
ggggacaagtttgtacaaaaaagcaggcttcgccaccatgtgtcaccagcagttg

SEQ ID NO: 127 - DNA, 51 bp, Artificial Sequence
  GCP Reverse Primer
ggggaccactttgtacaagaaagctgggtttcaatggtgatggtgatggtg

SEQ ID NO: 128 - DNA, 18 bp, Artificial Sequence
  SP6 Primer
atttaggtgacactatag

SEQ ID NO: 129 - DNA, 20 bp, Artificial Sequence
  pEAK12F Primer
gccagcttggcacttgatgt

SEQ ID NO: 130 - DNA, 20 bp, Artificial Sequence
  pEAK12R Primer
gatggaggtggacgtgtcag

SEQ ID NO: 131 - DNA, 18 bp, Artificial Sequence
  21M13 Primer
tgtaaaacgacggccagt

SEQ ID NO: 132 - DNA, 18 bp, Artificial Sequence
  M13REV Primer
caggaaacagctatgacc

SEQ ID NO: 133 - DNA, 20 bp, Artificial Sequence
  INSP088-CP1 Primer
gagccgtagctcttccaata

SEQ ID NO: 134 - DNA, 20 bp, Artificial Sequence
  INSP088-CP2 Primer
gctggacttacctcattcac

SEQ ID NO: 135 - DNA, 19 bp, Artificial Sequence
  INSP088-CP3 Primer
gtggtcatccagaggcttt

SEQ ID NO: 136 - DNA, 20 bp, Artificial Sequence
  INSP088-SP1 Primer
ggacagcaatgaaccatgtg

SEQ ID NO: 137 - DNA, 20 bp, Artificial Sequence
  INSP088-SP2 Primer
cacagcaatggctcatacag

SEQ ID NO: 138 - DNA, 20 bp, Artificial Sequence
  INSP088-SP3 Primer
agccagaaacctactccatc

SEQ ID NO: 139 - DNA, 19 bp, Artificial Sequence
  T7 Primer
taatacgactcactatagg

SEQ ID NO: 140 - DNA, 18 bp, Artificial Sequence
  T3 Primer
attaaccctcactaaagg

(19) United States
(12) Patent Application Publication
(10) Pub. No.: US 2007/0021588 A1
(43) Pub. Date: Jan. 25, 2007

(54) Alpha macroglobulin protein family members

(76) Inventors: Richard Joseph Fagan, LONDON (GB); Christopher Benjamin Phelps, London (GB); Tania Maria Rodrigues, London (GB); Christine Power, Thoiry (FR); Jadwiga Bienkowska, Cambridge, MA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/531,751

(22) PCT Filed: Oct. 16, 2003
(86) PCT No.: PCT/GB03/04500

§ 371 Date: Dec. 22, 2005

(30) Foreign Application Priority Data
Oct. 16, 2002 (GB) 0224116.4

(51) Int. Cl. C07K 14/00; C07K 1/00; C07K 17/00

(52) U.S. Cl. 530/350

(57) ABSTRACT

This invention relates to two proteins, termed INSP087, herein identified as members of the alpha macroglobulin family, in particular as alpha-2-macroglobulin-like proteinase inhibitors, and to the use of these proteins and nucleic acid sequences from the encoding genes in the diagnosis, prevention, and treatment of disease.

This invention relates to novel proteins, termed INSP087 and INSP088, herein identified as members of the alpha macroglobulin family, in particular as alpha-2-macroglobulin-like proteinase inhibitors, and to the use of these proteins and nucleic acid sequences from the encoding genes in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

Alpha Macroglobulin Family

The alpha macroglobulin family of proteins is divided into two general divisions—the alpha-2-macroglobulin like proteins and the complement-like proteins—that are thought to have arisen from a common ancestral alpha-2-macroglobulin-like molecule (Lin et al, 2002). The alpha macroglobulin family is therefore also known as the α2M/C3,C4,C5 family of thioester-containing protease inhibitor and complement proteins. A new member of the macroglobulin family, CD109, has recently been identified which has not yet been characterised as belonging to either the alpha-2-macroglobulin-like division or the complement-like division.

Alpha-2-Macroglobulin-Like Proteins:

The alpha-2-macroglobulin-like proteins are large glycoproteins which act as non-specific irreversible proteinase inhibitors and which are found in the plasma of vertebrates, in the hemolymph of invertebrates such as lobster and in bird and reptile egg whites (Sottrup-Jensen L et al, 1989, J Biol Chem. 264(20):11539-42).

In humans, alpha-2-macroglobulin-like proteins include human alpha-2-macroglobulin and human Pregnancy Zone Protein (PZP). These proteinase inhibitors play a vital role in the clearance of proteinases from the circulation and in regulating proteinase activity in fibrinolysis, coagulation and complement activation.

Human alpha-2-macroglobulin is the largest known proteinase inhibitor (Mᵣ=720,000). It is a homotetramer formed by two protomeric units, each of which contains two 180-kDa subunits linked by two disulfide bridges. (Qazi et al, 1998, J Biol Chem. 273(15):8987-93. Each subunit of human alpha-2-macroglobulin has a bait region of approximately 40 amino acid residues, an internal thiol ester bond and a receptor-binding domain. Cleavage of the bait region by an attacking proteinase causes activation and cleavage of the internal thiol ester bond. This triggers major structural changes in the alpha-2-macroglobulin, known as the “mouse trap mechanism”, which result in the proteinase being entrapped by and covalently linked to the alpha-2-macroglobulin. Formation of this alpha-2-macroglobulin-proteinase complex results in the exposure of the receptor binding domain of the alpha-2-macroglobulin and engagement of the receptor binding domain by cell-membrane receptors permits clearance of the alpha-2-macroglobulin-proteinase complex from circulation, via endocytosis. In contrast to the mode of inhibition of all other natural proteinase inhibitors, the entrapped proteinase retains its catalytic activity. Although inaccessible to its target proteins, the entrapped proteinase may react with small substrates and inhibitors (Qazi et al, 1998, supra).

Rats contain at least three different alpha-2-macroglobulin-like proteins, alpha 2-macroglobulin, alpha 1 inhibitor III and alpha 1-macroglobulin, which act as broad range proteinase inhibitors using a similar mechanism to known human alpha-2-macroglobulin-like proteins (Eggertsen G et al 1991). Chickens contain an alpha-2-macroglobulin-like protein, ovostatin, in egg white. Ovostatin differs from the alpha-2-macroglobulin-like proteins found in humans and rats in that it is more substrate specific, inhibiting only metalloproteinases stoichiometrically. Furthermore, ovostatin lacks the thiol ester bond that other family members possess so that its mechanism of action does not involve establishing a covalent linkage between ovastatin and the proteinase (Nagase et al, 1986, J Biol Chem. 261(3):1421-6.)

Alpha-2-macroglobulin-like proteinase inhibitors have been implicated in a number of diseases in humans. Alterations in the serum level of human alpha-2-macroglobulin and pregnancy zone protein have been suggested to be indicative of a number of diseases and disorders. Decreased alpha-2-macroglobulin concentration typically results from enhanced clearance of alpha-2-macroglobulin-proteinase complex and occurs in states of increased proteolytic activity, such as pancreatitis. Increased serum alpha-2-macroglobulin is frequently seen in nephrotic conditions (Petersen, 1993, Dan Med Bull. 40(4):409-46), and it has been shown that proteinase inhibitory activity is lower in patients with idiopathic nephrotic syndrome (Asami et al, 1996, Nephron 72(4):512-7). Increased serum levels of Pregnancy Zone Protein may be an indication of threatened abortion, as well as trophoblastic diseases and gynaecological tumours (Teng H et al, 1994, Chin Med J (Engl) 107(12):910-4). Furthermore, pregnancy zone protein and alpha-2-macroglobulin are both able to interact with Trypanosoma cruzi proteinases and it has been suggested that they could prevent or minimize harmful action of T. cruzi proteinases, such as cruzipain, on human host molecules and regulate parasite functions controlled by cruzipain (Ramos et al, 2002, Exp Parasitol. 100(2):121-30.)

A number of studies have linked a valine to isoleucine (Val1000Ile) polymorphism in human alpha-2-macroglobulin with argyrophilic grain disease (AGD), a neurodegenerative disorder of the aged human brain associated with the formation of abnormal tau protein in specific neurones and macroglial cells (Ghebremedhin E et al, 2002, Neuropathol Appl Neurobiol (4):308-13), Alzheimer's Disease and Parkinson's Disease (Tang G et al, 2002, Neurosci Lett 328(2):195-7; Zappia et al, 2002, Neurology 59(5):756-8). However, other studies have suggested that this polymorphism does not represent a risk factor for Parkinson's Disease (Nicoletti G et al, 2002, Neurosci Lett 328(1):65-7).

Complement-Like Proteins

Complement components C3, C4 and C5 are focal points in the complement system, each interacting with numerous other components during complement activation, regulation, and receptor-mediated functions. These proteins are involved in a wide variety of biological activities such as in innate response and host defence (Fritzinger et al, 1992, J. Immunol. 149: 3554-3562).

C3, C4 and C5 belong to the alpha macroglobulin family but contain specific features that are not present in alpha-2-macroglobulin-like proteins, including an anaphylatoxin domain, a C-terminal netrin (NTR) domain and stretches of basic residues for proteolytic processing to form multiple chain structures. (Martinez et al, 2001, Front Biosci 1; 6:D904-13). Activation of C3, C4 and C5 leads to enzymatic cleavage producing fragments C3a, C4a and C5a (Ogata et al, 1989, J. Biol. Chem. 264: 16565-16572). Each a-fragment forms a distinct structural domain of approximately 76 amino acids, coded for by a single exon within its respective complement protein gene. (Ogata et al, 1989, supra; Gennaro et al, 1986, Eur. J. Biochem. 155: 77-86). The fragments are highly hydrophilic, with a mainly helical structure held together by 3 disulphide bridges (Gennaro et al, 1986, supra). The fragments are anaphylatoxins, causing smooth muscle contraction, histamine release from mast cells, and enhanced vascular permeability (Gennaro et al, 1986, supra). They also mediate chemotaxis, inflammation, and generation of cytotoxic oxygen radicals (Kohl, 2001, Mol Immunol 38(2-3):175-87).

The C-terminal netrin (NTR) domain of C3, C4 and C5 (also known as the C345C module) is also found in other proteins such as the netrins and tissue inhibitor metalloproteases (TIMPs). The functional role of NTR domains is generally unknown with the exception of TIMPs, where the NTR domain is known to be a binding site for the metalloproteinase and C5, where the NTR domain is known to be a binding site for the CP convertase, an enzyme responsible for proteolytic processing (Sandoval et al, 2000, J Immunol 165(2):1066-73).

Complement proteins and C3, C4 and C5 in particular, have been implicated in a variety of diseases and disorders. Generally, the anaphylatoxins formed by cleavage of C3, C4 and C5 may play a role in sepsis, immune complex disease, delayed type hypersensitivity and asthma. More specifically, C5a has been found to exert an anti-inflammatory effect in acute pancreatitis and associated lung injury (Bhatia M et al, 2001, Am J Physiol Gastrointest Liver Physiol 280(5):G974-8) but to induce a chronic microglia-mediated focal inflammatory response in Alzheimer's Disease (O'Barr S et al, 2000, J Neuroimmunol 109(2):87). Complement proteins also appear to play a role in the pathophysiology of ischaemic heart diseases and it has been suggested that complement inhibitors might be used in the treatment of this disease (Sheman S K et al, 2001, BioDrugs 15(9):595-607). It has also been suggested that the C4 genes may be the disease-predisposing genes connected to susceptibility to Psoriasis vulgaris (Cislo et al, 2002, Immunol Lett 2002 80(3):145-9).

CD109

CD109 is a new member of the alpha macroglobulin family whose function remains largely unknown (Lin et al., 2002, Blood 99(5):1683-91). In terms of sequence similarity, it appears to be closely related to alpha-2-macroglobulin-like proteins and more distantly related to C3 and C4 proteins. However, CD109 differs from typical alpha-2-macroglobulin-like proteinase inhibitors in several respects. Unlike alpha-2-macroglobulin-like proteinase inhibitors which generally exist as tetramers, CD109 exists as a monomer. CD109 does not contain a receptor binding domain present in alpha-2-macroglobulin-like proteinase inhibitors (Nielsen et al, 1996, J Biol Chem 271(22):12909-12) and unlike alpha-2-macroglobulin-like proteinase inhibitors, CD109 is membrane bound through a GPI linker. Furthermore, although CD109 contains an thioester bond similar to that found in alpha-2-macroglobulin-like proteinase inhibitors, its chemical reactivity resembles that of complement proteins. It is therefore unclear which division this novel member of the alpha macroglobulin family belongs to.

Increasing knowledge of the alpha macroglobulin family is of extreme importance in increasing the understanding of the underlying pathways that lead to the disease states and associated disease states mentioned above, and in developing more effect gene and/or drug therapies to treat these disorders. In particular, increasing knowledge of the alpha-2-macroglobulin-like proteinase inhibitors is of importance in understanding the disease states in which these proteins are implicated and developing therapies to treat these disorders.

The Invention

The invention is based on the discovery that the INSP087 and INSP088 proteins function as alpha macroglobulins and in particular as alpha-2-macroglobulin-like proteinase inhibitors.

In one embodiment of the first aspect of the invention, there is provided a polypeptide which:
(i) comprises the amino acid sequence as recited in SEQ ID NO:2, SEQ ID NO:4, SEQ ID NO: 6, SEQ ID NO:8, SEQ ID NO:10, SEQ ID NO:12, SEQ ID NO:14, SEQ ID NO:16, SEQ IS NO:18, SEQ ID NO:20, SEQ ID NO:22, SEQ ID NO:24, SEQ ID NO:26, SEQ ID NO:28, SEQ ID NO:30, SEQ ID NO: 32, SEQ ID NO:34, SEQ ID NO:36, SEQ ID NO:38, SEQ ID NO:40, SEQ ID NO: 42, SEQ ID NO:44, SEQ ID NO:46, SEQ ID NO:48, SEQ ID NO: 50, SEQ ID NO: 52, SEQ ID NO:54, SEQ ID NO:56, SEQ ID NO:58, SEQ ID NO:60, SEQ ID NO:62, SEQ ID NO:64, SEQ ID NO:66 and/or SEQ ID NO:68;
(ii) is a fragment thereof which functions as an alpha-2-macroglobulin-like proteinase inhibitor, or has an antigenic determinant in common with a polypeptide according to (i); or
(iii) is a functional equivalent of (i) or (ii).

Preferably, the polypeptide according to this first embodiment of this first aspect of the invention:
(i) comprises the amino acid sequence as recited in SEQ ID NO:68;
(ii) is a fragment thereof which functions as an alpha-2-macroglobulin-like proteinase inhibitor, or has an antigenic determinant in common with a polypeptide according to (i); or
(iii) is a functional equivalent of (i) or (ii).

According to a second embodiment of this first aspect of the invention, there is provided a polypeptide which:
(i) consists of the amino acid sequence as recited in SEQ ID NO:2, SEQ ID. NO:4, SEQ ID NO: 6, SEQ ID NO:8, SEQ ID NO:10, SEQ ID NO:12, SEQ ID NO:14, SEQ ID NO:16, SEQ IS NO:18, SEQ ID NO:20, SEQ ID NO:22, SEQ ID NO:24, SEQ ID NO:26, SEQ ID NO:28, SEQ ID NO:30, SEQ ID NO: 32, SEQ ID NO:34, SEQ ID NO:36, SEQ ID NO:38, SEQ ID NO:40, SEQ ID NO: 42, SEQ ID NO:44, SEQ ID NO:46, SEQ ID NO:48, SEQ ID NO: 50, SEQ ID NO: 52, SEQ ID NO:54, SEQ ID NO:56, SEQ ID NO:58, SEQ ID NO:60, SEQ ID NO:62, SEQ ID NO:64, SEQ ID NO:66 and/or SEQ ID NO:68;
(ii) is a fragment thereof which functions as an alpha-2-macroglobulin-like proteinase inhibitor, or has an antigenic determinant in common with a polypeptide according to (i); or
(iii) is a functional equivalent of (i) or (ii).

The polypeptide having the sequence recited in SEQ ID NO:2 is referred to hereafter as “the INSP087 exon 1 polypeptide”. The polypeptide having the sequence recited in SEQ ID NO:4 is referred to hereafter as “the INSP087 exon 2 polypeptide”. The polypeptide having the sequence recited in SEQ ID NO:6 is referred to hereafter as “the INSP087 exon 3 polypeptide”. The polypeptide having the sequence recited in SEQ ID NO:8 is referred to hereafter as “the INSP087 exon 4 polypeptide”. The polypeptide having the sequence recited in SEQ ID NO:10 is referred to hereafter as “the INSP087 exon 5 polypeptide”. The polypeptide having the sequence recited in SEQ ID NO:12 is referred to hereafter as “the INSP087 exon 6 polypeptide”. The polypeptide having the sequence recited in SEQ ID NO:14 is referred to hereafter as “the INSP087 exon 7 polypeptide”. The polypeptide having the sequence recited in SEQ ID NO:16 is referred to hereafter as “the INSP087 exon 8 polypeptide”. The polypeptide having the sequence recited in SEQ ID NO:18 is referred to hereafter as “the INSP087 exon 9 polypeptide”. The polypeptide having the sequence recited in SEQ ID NO:20 is referred to hereafter as “the INSP087 exon 10 polypeptide”. The polypeptide having the sequence recited in SEQ ID NO:22 is referred to hereafter as “the INSP087 exon 11 polypeptide”. The polypeptide having the sequence recited in SEQ ID NO:24 is referred to hereafter as “the INSP087 exon 12 polypeptide”. The polypeptide having the sequence recited in SEQ ID NO:26 is referred to hereafter as “the INSP087 exon 13 polypeptide”. The polypeptide having the sequence recited in SEQ ID NO:28 is referred to hereafter as “the INSP087 exon 14 polypeptide”. The polypeptide having the sequence recited in SEQ ID NO:30 is referred to hereafter as “the INSP087 exon 15 polypeptide”. The polypeptide having the sequence recited in SEQ ID NO:32 is referred to hereafter as “the INSP087 exon 16 polypeptide”. The polypeptide having the sequence recited in SEQ ID NO:34 is referred to hereafter as “the INSP087 exon 17 polypeptide”. The polypeptide having the sequence recited in SEQ ID NO:36 is referred to hereafter as “the INSP087 exon 18 polypeptide”. The polypeptide having the sequence recited in SEQ ID NO:38 is referred to hereafter as “the INSP087 exon 19 polypeptide”. The polypeptide having the sequence recited in SEQ ID NO:40 is referred to hereafter as “the INSP087 exon 20 polypeptide”. The polypeptide having the sequence recited in SEQ ID NO:42 is referred to hereafter as “the INSP087 exon 21 polypeptide”. The polypeptide having the sequence recited in SEQ ID NO:44 is referred to hereafter as “the INSP087 exon 22 polypeptide”. The polypeptide having the sequence recited in SEQ ID NO:46 is referred to hereafter as “the INSP087 exon 23 polypeptide”. The polypeptide having the sequence recited in SEQ ID NO:48 is referred to hereafter as “the INSP087 exon 24 polypeptide”. The polypeptide having the sequence recited in SEQ ID NO:50 is referred to hereafter as “the INSP087 exon 25 polypeptide”. The polypeptide having the sequence recited in SEQ ID NO:52 is referred to hereafter as “the INSP087 exon 26 polypeptide”. The polypeptide having the sequence recited in SEQ ID NO:54 is referred to hereafter as “the INSP087 exon 27 polypeptide”. The polypeptide having the sequence recited in SEQ ID NO:56 is referred to hereafter as “the INSP087 exon 28 polypeptide”. The polypeptide having the sequence recited in SEQ ID NO:58 is referred to hereafter as “the INSP087 exon 29 polypeptide”. The polypeptide having the sequence recited in SEQ ID NO:60 is referred to hereafter as “the INSP087 30 exon polypeptide”. The polypeptide having the sequence recited in SEQ ID NO:62 is referred to hereafter as “the INSP087 exon 31 polypeptide”. The polypeptide having the sequence recited in SEQ ID NO:64 is referred to hereafter as “the INSP087 exon 32 polypeptide”. The polypeptide having the sequence recited in SEQ ID NO:66 is referred to hereafter as “the INSP087 exon 33 polypeptide”.

The polypeptide having the sequence recited in SEQ ID NO: 68 is referred to hereafter as “the INSP087 partial polypeptide”. It is anticipated that the INSP087 partial polypeptide forms part of a larger polypeptide. As there is no methionine start codon at the start of the INSP087 partial polypeptide (SEQ ID NO:68), it is considered likely that the full-length INSP087 polypeptide contains additional amino acids 5′ of the partial sequence recited in SEQ ID NO:68.

The term “INSP087 polypeptides” as used herein includes polypeptides comprising or consisting of the INSP087 exon 1 polypeptide, the INSP087 exon 2 polypeptide, the INSP087 exon 3 polypeptide, the INSP087 exon 4 polypeptide, the INSP087 exon 5 polypeptide, the INSP087 exon 6 polypeptide, the INSP087 exon 7 polypeptide, the INSP087 exon 8 polypeptide, the INSP087 exon 9 polypeptide, the INSP087 exon 10 polypeptide, the INSP087 exon 11 polypeptide, the INSP087 exon 12 polypeptide, the INSP087 exon 13 polypeptide, the INSP087 exon 14 polypeptide, the INSP087 exon 15 polypeptide, the INSP087 exon 16 polypeptide, the INSP087 exon 17 polypeptide, the INSP087 exon 18 polypeptide, the INSP087 exon 19 polypeptide, the INSP087 exon 20 polypeptide, the INSP087 exon 21 polypeptide, the INSP087 exon 22 polypeptide, the INSP087 exon 23 polypeptide, the INSP087 exon 24 polypeptide, the INSP087 exon 25 polypeptide, the INSP087 exon 26 polypeptide, the INSP087 exon 27 polypeptide, the INSP087 exon 28 polypeptide, the INSP087 exon 29 polypeptide, the INSP087 exon 30 polypeptide, the INSP087 exon 31 polypeptide, the INSP087 exon 32 polypeptide, the INSP087 exon 33 polypeptide and the INSP087 partial polypeptide.

In a third embodiment of the first aspect of the invention, there is provided a polypeptide which:
(i) comprises the amino acid sequence as recited in SEQ ID NO:70, SEQ ID NO:72, SEQ ID NO: 74, SEQ ID NO:76, SEQ ID NO:78, SEQ ID NO:80, SEQ ID NO:82, SEQ ID NO:84, SEQ IS NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, and/or SEQ ID NO:112;
(ii) is a fragment thereof which functions as an alpha-2-macroglobulin-like proteinase inhibitor, or has an antigenic determinant in common with a polypeptide according to (i); or
(iii) is a functional equivalent of (i) or (ii).

Preferably, the polypeptide according to this third embodiment of the first aspect of the invention:
(i) comprises the amino acid sequence as recited in SEQ ID NO:112;
(ii) is a fragment thereof which functions as an alpha-2-macroglobulin-like proteinase inhibitor, or has an antigenic determinant in common with a polypeptide according to (i); or
(iii) is a functional equivalent of (i) or (ii).

In a fourth embodiment of the first aspect of the invention, there is provided a polypeptide which:
(i) consists of the amino acid sequence as recited in SEQ ID NO:70, SEQ ID NO:72, SEQ ID NO: 74, SEQ ID NO:76, SEQ ID NO:78, SEQ ID NO:80, SEQ ID NO:82, SEQ ID NO:84, SEQ IS NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, and/or SEQ ID NO:112;
(ii) is a fragment thereof which functions as an alpha-2-macroglobulin-like proteinase inhibitor, or has an antigenic determinant in common with a polypeptide according to (i); or
(iii) is a functional equivalent of (i) or (ii).

The polypeptide having the sequence recited in SEQ ID NO:70 is referred to hereafter as “the INSP088 exon 1 polypeptide”. The polypeptide having the sequence recited in SEQ ID NO:72 is referred to hereafter as “the INSP088 exon 2 polypeptide”. The polypeptide having the sequence recited in SEQ ID NO:74 is referred to hereafter as “the INSP088 exon 3 polypeptide”. The polypeptide having the sequence recited in SEQ ID NO:76 is referred to hereafter as “the INSP088 exon 4 polypeptide”. The polypeptide having the sequence recited in SEQ ID NO:78 is referred to hereafter as “the INSP088 exon 5 polypeptide”. The polypeptide having the sequence recited in SEQ ID NO:80 is referred to hereafter as “the INSP088 exon 6 polypeptide”. The polypeptide having the sequence recited in SEQ ID NO:82 is referred to hereafter as “the INSP088 exon 7 polypeptide”. The polypeptide having the sequence recited in SEQ ID NO:84 is referred to hereafter as “the INSP088 exon 8 polypeptide”. The polypeptide having the sequence recited in SEQ ID NO:86 is referred to hereafter as “the INSP088 exon 9 polypeptide”. The polypeptide having the sequence recited in SEQ ID NO:88 is referred to hereafter as “the INSP088 exon 10 polypeptide”. The polypeptide having the sequence recited in SEQ ID NO:90 is referred to hereafter as “the INSP088 exon 11 polypeptide”. The polypeptide having the sequence recited in SEQ ID NO:92 is referred to hereafter as “the INSP088 exon 12 polypeptide”. The polypeptide having the sequence recited in SEQ ID NO:94 is referred to hereafter as “the INSP088 exon 13 polypeptide”. The polypeptide having the sequence recited in SEQ ID NO:96 is referred to hereafter as “the INSP088 exon 14 polypeptide”. The polypeptide having the sequence recited in SEQ ID NO:98 is referred to hereafter as “the INSP088 exon 15 polypeptide”. The polypeptide having the sequence recited in SEQ ID NO:100 is referred to hereafter as “the INSP088 exon 16 polypeptide”. The polypeptide having the sequence recited in SEQ ID NO:102 is referred to hereafter as “the INSP088 exon 17 polypeptide”. The polypeptide having the sequence recited in SEQ ID NO:104 is referred to hereafter as “the INSP088 exon 18 polypeptide”. The polypeptide having the sequence recited in SEQ ID NO:106 is referred to hereafter as “the INSP088 exon 19 polypeptide”. The polypeptide having the sequence recited in SEQ ID NO:108 is referred to hereafter as “the INSP088 exon 20 polypeptide”. The polypeptide having the sequence recited in SEQ ID NO:110 is referred to hereafter as “the INSP088 exon 21 polypeptide”.

The polypeptide having the sequence recited in SEQ ID NO:112 is referred to hereafter as “the INSP088 partial polypeptide”. It is anticipated that the INSP088 partial polypeptide forms part of a larger polypeptide. As there is no methionine start codon at the start of the INSP087 partial polypeptide (SEQ ID NO:112), it is considered likely that the full-length INSP087 polypeptide contains additional amino acids 5′ of the partial sequence recited in SEQ ID NO:112.

The term “INSP088 polypeptides” as used herein includes polypeptides comprising or consisting of the INSP088 exon 1 polypeptide, the INSP088 exon 2 polypeptide, the INSP088 exon 3 polypeptide, the INSP088 exon 4 polypeptide, the INSP088 exon 5 polypeptide, the INSP088 exon 6 polypeptide, the INSP088 exon 7 polypeptide, the INSP088 exon 8 polypeptide, the INSP088 exon 9 polypeptide, the INSP088 exon 10 polypeptide, the INSP088 exon 11 polypeptide, the INSP088 exon 12 polypeptide, the INSP088 exon 13 polypeptide, the INSP088 exon 14 polypeptide, the INSP088 exon 15 polypeptide, the INSP088 exon 16 polypeptide, the INSP088 exon 17 polypeptide, the INSP088 exon 18 polypeptide, the INSP088 exon 19 polypeptide, the INSP088 exon 20 polypeptide, the INSP088 exon 21 polypeptide and the INSP088 partial polypeptide.

By “functions as an alpha-2-macroglobulin-like proteinase inhibitor” we refer to polypeptides that comprise amino acid sequence or structural features that can be identified as conserved features within alpha-2-macroglobulin-like proteinase inhibitors, such that the polypeptide's interaction with a proteinase is not substantially affected detrimentally in comparison to the function of the full length wild type polypeptide. Examples of assays which may be used to determine the biological activity of a polypeptide of the invention are described in Example 6.

The receptor binding domain of INSP087 has been identified as being located within amino acid residues 1263-1357 of the INSP087 partial polypeptide (SEQ ID NO:68). The amino acid sequence of the receptor binding domain of INSP087 is recited herein as SEQ ID NO:113. The receptor binding domain of INSP087 has been cloned, as described in Example 3. The sequence of the cloned receptor binding domain of INSP087, which differs from by just one amino acid residue from SEQ ID NO:113, is given herein as SEQ ID NO:115. A preferred polypeptide fragment according to the first aspect of the invention therefore comprises or consists of SEQ ID NO:113 or SEQ ID NO:115 or is a functional equivalent thereof. It is considered highly likely, however that the receptor binding domain will fold correctly and show biological activity if additional residues C terminal and/or N terminal of these boundaries in the polypeptide sequence are included in the polypeptide fragment. For example, an additional 5, 10, 20, 30, 40, 50, 100 or even as many as 200 amino acid residues from the INSP087 polypeptide sequence, or from a homologous sequence, may be included at either or both the C terminal and/or N terminal of the boundaries of the receptor binding domain, without prejudicing the ability of the polypeptide fragment to fold correctly and exhibit receptor binding domain activity. Extensions as large as 100 or 200 residues may be necessary due to the presence of large loops between secondary structural elements.

For truncated variants of the INSP087 receptor binding domain, one or a few amino acid residues (for example, 2, 3, 4, 5, 10, 15, 20, 25, 30 or more) may be deleted at either or both the C terminus or the N terminus of the domain without prejudicing biological activity.

The receptor binding domain of INSP088 has been identified as being located within amino acid residues 803-894 of the INSP088 partial polypeptide (SEQ ID NO:112). A preferred polypeptide fragment according to the first aspect of the invention therefore comprises or consists of amino acid residues 803-894 of SEQ ID NO:112 or is a functional equivalent thereof. The amino acid sequence of the receptor binding domain of INSP088 is recited herein SEQ ID NO:117. The receptor binding domain of INSP087 has been cloned, as described in Example 4, confirming the sequence is correct. A preferred polypeptide fragment according to the first aspect of the invention therefore comprises or consists of SEQ ID NO:117 or is a functional equivalent thereof. It is considered highly likely, however that the receptor binding domain will fold correctly and show biological activity if additional residues C terminal and/or N terminal of these boundaries in the polypeptide sequence are included in the polypeptide fragment. For example, an additional 5, 10, 20, 30, 40, 50, 100 or even as many as 200 amino acid residues from the INSP088 polypeptide sequence, or from a homologous sequence, may be included at either or both the C terminal and/or N terminal of the boundaries of the receptor binding domain, without prejudicing the ability of the polypeptide fragment to fold correctly and exhibit receptor binding domain activity. Extensions as large as 100 or 200 residues may be necessary due to the presence of large loops between secondary structural elements.

For truncated variants of the INSP088 receptor binding domain, one or a few amino acid residues (for example, 2, 3, 4, 5, 10, 15, 20, 25, 30 or more) may be deleted at either or both the C terminus or the N terminus of the domain without prejudicing biological activity.

The macroglobulin domain of INSP088 has been identified as being located within amino acid residues 163-894 of the INSP088 partial polypeptide (SEQ ID NO:112). This amino acid sequence is recited herein as SEQ ID NO:119. A preferred polypeptide fragment according to the first aspect of the invention therefore comprises or consists of amino acid of SEQ ID NO:119 or is a functional equivalent thereof. It is considered highly likely, however that the macroglobulin domain will fold correctly and show biological activity if additional residues C terminal and/or N terminal of these boundaries in the polypeptide sequence are included in the polypeptide fragment. For example, an additional 5, 10, 20, 30, 40, 50, 100 or even as many as 200 amino acid residues from the INSP088 polypeptide sequence, or from a homologous sequence, may be included at either or both the C terminal and/or N terminal of the boundaries of the macroglobulin domain, without prejudicing the ability of the polypeptide fragment to fold correctly and exhibit macroglobulin domain activity. Extensions as large as 100 or 200 residues may be necessary due to the presence of large loops between secondary structural elements.

For truncated variants of the INSP088 macroglobulin domain, one or a few amino acid residues (for example, 2, 3, 4, 5, 10, 15, 20, 25, 30 or more) may be deleted at either or both the C terminus or the N terminus of the domain without prejudicing biological activity.

A further preferred polypeptide fragment according to the first aspect of the invention comprises or consists of SEQ ID NO:121, a fragment of the INSP088 partial polypeptide including the receptor binding domain and the macroglobulin domain, cloned from human tissue as described in Example 5.

In a second aspect, the invention provides a purified nucleic acid molecule which encodes a polypeptide of the first aspect of the invention.

In a first embodiment of this aspect of the invention, the purified nucleic acid molecule comprises the nucleic acid sequence as recited in SEQ ID NO:1 (encoding the INSP087 exon 1 polypeptide), SEQ ID NO:3 (encoding the INSP087 exon 2 polypeptide), SEQ ID NO:5 (encoding the INSP087 exon 3 polypeptide), SEQ ID NO:7 (encoding the INSP087 exon 4 polypeptide), SEQ ID NO:9 (encoding the INSP087 exon 5 polypeptide), SEQ ID NO:11 (encoding the INSP087 exon 6 polypeptide), SEQ ID NO:13 (encoding the INSP087 exon 7 polypeptide), SEQ ID NO:15 (encoding the INSP087 exon 8 polypeptide), SEQ ID NO:17 (encoding the INSP087 exon 9 polypeptide), SEQ ID NO:19 (encoding the INSP087 exon 10 polypeptide), SEQ ID NO:21 (encoding the INSP087 exon 11 polypeptide), SEQ ID NO:23 (encoding the INSP087 exon 12 polypeptide), SEQ ID NO:25 (encoding the INSP087 exon 13 polypeptide), SEQ ID NO:27 (encoding the INSP087 exon 14 polypeptide), SEQ ID NO:29 (encoding the INSP087 exon 15 polypeptide), SEQ ID NO:31 (encoding the INSP087 exon 16 polypeptide), SEQ ID NO:33 (encoding the INSP087 exon 17 polypeptide), SEQ ID NO:35 (encoding the INSP087 exon 18 polypeptide), SEQ ID NO:37 (encoding the INSP087 exon 19 polypeptide), SEQ ID NO:39 (encoding the INSP087 exon 20 polypeptide), SEQ ID NO:41 (encoding the INSP087 exon 21 polypeptide), SEQ ID NO:43 (encoding the INSP087 exon 22 polypeptide), SEQ ID NO:45 (encoding the INSP087 exon 23 polypeptide), SEQ ID NO:47 (encoding the INSP087 exon 24 polypeptide), SEQ ID NO:49 (encoding the INSP087 exon 25 polypeptide), SEQ ID NO:51 (encoding the INSP087 exon 26 polypeptide), SEQ ID NO:53 (encoding the INSP087 exon 27 polypeptide), SEQ ID NO:55 (encoding the INSP087 exon 28 polypeptide), SEQ ID NO:57 (encoding the INSP087 exon 29 polypeptide), SEQ ID NO:59 (encoding the INSP087 exon 30 polypeptide), SEQ ID NO:61 (encoding the INSP087 exon 31 polypeptide), SEQ ID NO:63 (encoding the INSP087 exon 32 polypeptide), SEQ ID NO:65 (encoding the INSP087 exon 33 polypeptide), SEQ ID NO:67 (encoding the INSP087 partial polypeptide) or is a redundant equivalent or fragment of any one of these sequences.

The invention further provides that the purified nucleic acid molecule consists of the nucleic acid sequence as recited in SEQ ID NO:1 (encoding the INSP087 exon 1 polypeptide), SEQ ID NO:3 (encoding the INSP087 exon 2 polypeptide), SEQ ID NO:5 (encoding the INSP087 exon 3 polypeptide), SEQ ID NO:7 (encoding the INSP087 exon 4 polypeptide), SEQ ID NO:9 (encoding the INSP087 exon 5 polypeptide), SEQ ID NO:11 (encoding the INSP087 exon 6 polypeptide), SEQ ID NO:13 (encoding the INSP087 exon 7 polypeptide), SEQ ID NO:15 (encoding the INSP087 exon 8 polypeptide), SEQ ID NO:17 (encoding the INSP087 exon 9 polypeptide), SEQ ID NO:19 (encoding the INSP087 exon 10 polypeptide), SEQ ID NO:21 (encoding the INSP087 exon 11 polypeptide), SEQ ID NO:23 (encoding the INSP087 exon 12 polypeptide), SEQ ID NO:25 (encoding the INSP087 exon 13 polypeptide), SEQ ID NO:27 (encoding the INSP087 exon 14 polypeptide), SEQ ID NO:29 (encoding the INSP087 exon 15 polypeptide), SEQ ID NO:31 (encoding the INSP087 exon 16 polypeptide), SEQ ID NO:33 (encoding the INSP087 exon 17 polypeptide), SEQ ID NO:35 (encoding the INSP087 exon 18 polypeptide), SEQ ID NO:37 (encoding the INSP087 exon 19 polypeptide), SEQ ID NO:39 (encoding the INSP087 exon 20 polypeptide), SEQ ID NO:41 (encoding the INSP087 exon 21 polypeptide), SEQ ID NO:43 (encoding the INSP087 exon 22 polypeptide), SEQ ID NO:45 (encoding the INSP087 exon 23 polypeptide), SEQ ID NO:47 (encoding the INSP087 exon 24 polypeptide), SEQ ID NO:49 (encoding the INSP087 exon 25 polypeptide), SEQ ID NO:51 (encoding the INSP087 exon 26 polypeptide), SEQ ID NO:53 (encoding the INSP087 exon 27 polypeptide), SEQ ID NO:55 (encoding the INSP087 exon 28 polypeptide), SEQ ID NO:57 (encoding the INSP087 exon 29 polypeptide), SEQ ID NO:59 (encoding the INSP087 exon 30 polypeptide), SEQ ID NO:61 (encoding the INSP087 exon 31 polypeptide), SEQ ID NO:63 (encoding the INSP087 exon 32 polypeptide), SEQ ID NO:65 (encoding the INSP087 exon 33 polypeptide), SEQ ID NO:67 (encoding the INSP087 partial polypeptide) or is a redundant equivalent or fragment of any one of these sequences.

In a second embodiment of this aspect of the invention, the purified nucleic acid molecule comprises the nucleic acid sequence as recited in SEQ ID NO:69 (encoding the INSP088 exon 1 polypeptide), SEQ ID NO:71 (encoding the INSP088 exon 2 polypeptide), SEQ ID NO:73 (encoding the INSP088 exon 3 polypeptide), SEQ ID NO:75 (encoding the INSP088 exon 4 polypeptide), SEQ ID NO:77 (encoding the INSP088 exon 5 polypeptide), SEQ ID NO:79 (encoding the INSP088 exon 6 polypeptide), SEQ ID NO:81 (encoding the INSP088 exon 7 polypeptide), SEQ ID NO:83 (encoding the INSP088 exon 8 polypeptide), SEQ ID NO:85 (encoding the INSP088 exon 9 polypeptide), SEQ ID NO:87 (encoding the INSP088 exon 10 polypeptide), SEQ ID NO:89 (encoding the INSP088 exon 11 polypeptide), SEQ ID NO:91 (encoding the INSP088 exon 12 polypeptide), SEQ ID NO:93 (encoding the INSP088 exon 13 polypeptide), SEQ ID NO:95 (encoding the INSP088 exon 14 polypeptide), SEQ ID NO:97 (encoding the INSP088 exon 15 polypeptide), SEQ ID NO:99 (encoding the INSP088 exon 16 polypeptide), SEQ ID NO:101 (encoding the INSP088 exon 17 polypeptide), SEQ ID NO:103 (encoding the INSP088 exon 18 polypeptide), SEQ ID NO:105 (encoding the INSP088 exon 19 polypeptide), SEQ ID NO:107 (encoding the INSP088 exon 20 polypeptide), SEQ ID NO:109 (encoding the INSP088 exon 21 polypeptide), SEQ ID NO:111 (encoding the INSP088 polypeptide) or is a redundant equivalent or fragment of any one of these sequences.

The invention further provides that the purified nucleic acid molecule consists of the nucleic acid sequence as recited in SEQ ID NO:69 (encoding the INSP088 exon 1 polypeptide), SEQ ID NO:71 (encoding the INSP088 exon 2 polypeptide), SEQ ID NO:73 (encoding the INSP088 exon 3 polypeptide), SEQ ID NO:75 (encoding the INSP088 exon 4 polypeptide), SEQ ID NO:77 (encoding the INSP088 exon 5 polypeptide), SEQ ID NO:79 (encoding the INSP088 exon 6 polypeptide), SEQ ID NO:81 (encoding the INSP088 exon 7 polypeptide), SEQ ID NO:83 (encoding the INSP088 exon 8 polypeptide), SEQ ID NO:85 (encoding the INSP088 exon 9 polypeptide), SEQ ID NO:87 (encoding the INSP088 exon 10 polypeptide), SEQ ID NO:89 (encoding the INSP088 exon 11 polypeptide), SEQ ID NO:91 (encoding the INSP088 exon 12 polypeptide), SEQ ID NO:93 (encoding the INSP088 exon 13 polypeptide), SEQ ID NO:95 (encoding the INSP088 exon 14 polypeptide), SEQ ID NO:97 (encoding the INSP088 exon 15 polypeptide), SEQ ID NO:99 (encoding the INSP088 exon 16 polypeptide), SEQ ID NO:101 (encoding the INSP088 exon 17 polypeptide), SEQ ID NO:103 (encoding the INSP088 exon 18 polypeptide), SEQ ID NO:105 (encoding the INSP088 exon 19 polypeptide), SEQ ID NO:107 (encoding the INSP088 exon 20 polypeptide), SEQ ID NO:109 (encoding the INSP088 exon 21 polypeptide), SEQ ID NO:11 (encoding the INSP088 polypeptide) or is a redundant equivalent or fragment of any one of these sequences.

Human ESTs BE144308 (sourced from head and neck tissue) and AK057908 (sourced from brain tissue) are specifically excluded from the scope of this aspect of the invention.

Preferred nucleic acid fragments according to the invention include fragments encoding the receptor binding domain of the INSP087 partial polypeptide and fragments encoding the receptor binding domain and/or macroglobulin domain of the INSP088 partial polypeptide. Preferred nucleic acid fragments comprise or consist of nucleotides SEQ ID NO:114 or SEQ ID NO:116 (encoding the INSP087 receptor binding domain), SEQ ID NO:118 (encoding the INSP088 receptor binding domain) or SEQ ID NO:120 (encoding the INSP088 macrogrlobulin domain). A further preferred nucleic acid fragment according to the invention comprises or consists of SEQ ID NO:122 (encoding the cloned INSP088 fragment having the amino acid sequence of SEQ ID NO:121).

In a third aspect, the invention provides a purified nucleic acid molecule which hydridizes under high stringency conditions with a nucleic acid molecule of the second aspect of the invention.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to alpha-2-macroglobulin-like proteinase inhibitors of the first aspect of the invention. Preferably, the ligand inhibits the function of a polypeptide of the first aspect of the invention.

Ligands to a polypeptide according to the invention may come in various forms including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

In a seventh aspect, the invention provides a compound that is effective to alter the expression of a natural gene which encodes a polypeptide of the first aspect of the invention or to regulate the activity of a polypeptide of the first aspect of the invention.

Such compounds may be identified using the assays and screening methods disclosed herein.

A compound of the seventh aspect of the invention may either increase (agonise) or decrease (antagonise) the level of expression of the gene or the activity of the polypeptide.

Importantly, the identification of the function of the INSP087 and INSP088 polypeptides allows for the design of screening methods capable of identifying compounds that are effective in the treatment and/or diagnosis of disease. Ligands and compounds according to the sixth and seventh aspects of the invention may be identified using such methods. Examples of suitable assays and screening methods are provided herein. These methods are included as aspects of the present invention.

In an eighth aspect, the invention provides a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, for use in therapy or diagnosis of a disease in which alpha-2-macroglobulin-like proteinase inhibitors are implicated. Such diseases and disorders may include reproductive disorders, cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, pancreatitis, arthritis, psoriasis, psoriasis vulgaris, respiratory tract inflammation, asthma, and organ transplant rejection; cardiovascular disorders, including hypertension, oedema, angina, atherosclerosis, thrombosis, sepsis, shock, reperfusion injury, and ischemia, particularly ischemic heart disease; neurological disorders including central nervous system disease, Alzheimer's disease, brain injury, Parkinson's disease, amyotrophic lateral sclerosis, and pain; developmental disorders; metabolic disorders including diabetes mellitus, osteoporosis, and obesity, AIDS, renal disease, particularly idiopathic nephrotic syndrome; lung injury; infections including viral infection, bacterial infection, fungal infection and parasitic infection, particularly Trypanosoma cruzi infection and other pathological conditions. Preferably, the disease is one in which alpha-2-macroglobulin-like proteinase inhibitors are implicated. These molecules may also be used in the manufacture of a medicament for the treatment of such disorders.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

Preferably, the disease diagnosed by a method of a ninth aspect of the invention is a disease in which alpha-2-macroglobulin-like proteinase inhibitors are implicated, as described above.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of the polypeptides of the first aspect of the invention as alpha-2-macroglobulin-like proteinase inhibitors. Suitable uses of the polypeptides of the invention as alpha-2-macroglobulin-like proteinase inhibitors include use as a diagnostic marker for a physiological or pathological condition selected from the list given above.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, for use in therapy or diagnosis. These molecules may also be used in the manufacture of a medicament for the treatment of a disease.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease. Preferably, the disease a disease in which alpha-2-macroglobulin-like proteinase inhibitors are implicated, as described above.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INSP087 and INSP088 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INSP087 and INSP088 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

Typically, greater than 30% identity between two polypeptides is considered to be an indication of functional equivalence. Preferably, functionally equivalent polypeptides of the first aspect of the invention have a degree of sequence identity with the INSP087 and INSP088 polypeptides, or with active fragments thereof, of greater than 45%. More preferred polypeptides have degrees of identity of greater than 50%, 60%, 70%, 80%, 90%, 95%, 98% or 99%, respectively.

The functionally-equivalent polypeptides of the first aspect of the invention may also be polypeptides which have been identified using one or more techniques of structural alignment. For example, the Inpharmatica Genome Threader™ technology that forms one aspect of the search tools used to generate the Biopendium search database may be used (see co-pending International Patent Application No. PCT/GB01/01105) to identify polypeptides of presently-unknown function which, while having low sequence identity as compared to the INSP087 and INSP088 polypeptides, are predicted to be alpha-2 macroglobulin-like proteinases, said method utilising a polypeptide of the first aspect of the invention, by virtue of sharing significant structural homology with the INSP087 and INSP088 polypeptide sequences. By “significant structural homology” is meant that the Inpharmatica Genome Threader™ predicts two proteins to share structural homology with a certainty of at 10% and above.

The polypeptides of the first aspect of the invention also include fragments of the INSP087 and INSP088 polypeptides and fragments of the functional equivalents of the INSP087 and INSP088 polypeptides, provided that those fragments retain alpha-2-macroglobulin-like proteinase inhibiting activity or have an antigenic determinant in common with the INSP087 and INSP088 polypeptides.

As used herein, the term “fragment” refers to a polypeptide having an amino acid sequence that is the same as part, but not all, of the amino acid sequence of the INSP087 and INSP088 polypeptides or one of its functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Fragments of the INSP087 and INSP088 polypeptides may consist of combinations of 2, 3, 4, 5, 6, 7, 8, 9, 10, 11, 12, 13, 14, 15, 16, 17, 18, 19 or 20 of neighbouring exon sequences in the INSP087 or INSP088 partial polypeptide sequences respectively.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

By “substantially greater affinity we mean that there is a measurable increase in the affinity for a polypeptide of the invention as compared with the affinity for known alpha-2-macroglobulin-like proteinase inhibitors.

Preferably, the affinity is at least 1.5-fold, 2-fold, 5-fold, 10-fold, 100-fold, 10³-fold, 10⁴-fold, 10⁵-fold or 10⁶-fold or greater for a polypeptide of the invention than for known alpha-2-macroglobulin-like proteinase inhibitors.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl. Acad. Sci. USA, 86, 10029 (1989); Gorman et al., Proc. Natl. Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode a polypeptide sequence as recited in SEQ ID NO:2, SEQ ID NO:4, SEQ ID NO: 6, SEQ ID NO:8, SEQ ID NO:10, SEQ ID NO:12, SEQ ID NO:14, SEQ ID NO:16, SEQ IS NO:18, SEQ ID NO:20, SEQ ID NO:22, SEQ ID NO:24, SEQ ID NO:26, SEQ ID NO:28, SEQ ID NO:30, SEQ ID NO: 32, SEQ ID NO:34, SEQ ID NO:36, SEQ ID NO:38, SEQ ID NO:40, SEQ ID NO: 42, SEQ ID NO:44, SEQ ID NO:46, SEQ ID NO:48, SEQ ID NO: 50, SEQ ID NO: 52, SEQ ID NO:54, SEQ ID NO:56, SEQ ID NO:58, SEQ ID NO:60, SEQ ID NO:62, SEQ ID NO:64, SEQ ID NO:66 SEQ ID NO:68; SEQ ID NO:70, SEQ ID NO:72, SEQ ID NO: 74, SEQ ID NO:76, SEQ ID NO:78, SEQ ID NO:80, SEQ ID NO:82, SEQ ID NO:84, SEQ IS NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, and SEQ ID NO:112, and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes a polypeptide of this invention may be identical to the coding sequence of one or more of the nucleic acid molecules disclosed herein.

These molecules also may have a different sequence which, as a result of the degeneracy of the genetic code, encode a polypeptide of in SEQ ID NO:2, SEQ ID NO:4, SEQ ID NO: 6, SEQ ID NO:8, SEQ ID NO:10, SEQ ID NO:12, SEQ ID NO:14, SEQ ID NO:16, SEQ IS NO:18, SEQ ID NO:20, SEQ ID NO:22, SEQ ID NO:24, SEQ ID NO:26, SEQ ID NO:28, SEQ ID NO:30, SEQ ID NO: 32, SEQ ID NO:34, SEQ ID NO:36, SEQ ID NO:38, SEQ ID NO:40, SEQ ID NO: 42, SEQ ID NO:44, SEQ ID NO:46, SEQ ID NO:48, SEQ ID NO: 50, SEQ ID NO: 52, SEQ ID NO:54, SEQ ID NO:56, SEQ ID NO:58, SEQ ID NO:60, SEQ ID NO:62, SEQ ID NO:64, SEQ ID NO:66 SEQ ID NO:68; SEQ ID NO:70, SEQ ID NO:72, SEQ ID NO: 74, SEQ ID NO:76, SEQ ID NO:78, SEQ ID NO:80, SEQ ID NO:82, SEQ ID NO:84, SEQ IS NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112.

Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used herein refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5× Denhardts solution, 10% dextan sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding the INSP087 or INSP088 and nucleic acid molecules that are substantially complementary to such nucleic acid molecules.

Preferably, a nucleic acid molecule according to this aspect of the invention comprises a region that is at least 80% identical over its entire length to such coding sequences, or is a nucleic acid molecule that is complementary thereto. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the INSP087 and INSP088 polypeptides.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP087 and INSP088 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP087 and INSP088 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109 or SEQ ID NO:111) are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfected or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., (supra). Particularly suitable methods include calcium phosphate transfection, DEAE-dextran mediated transfection, transvection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSport1™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk⁻ or aprt^{±} cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing on the surface thereof the polypeptide according to the first aspect of the invention, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by measuring the level of a signal generated from the interaction of the compound with the polypeptide.

A further preferred method for identifying an agonist or antagonist of a polypeptide of the invention comprises:

(a) contacting a cell expressing on the surface thereof the polypeptide, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying agonist or antagonist of a polypeptide of the present invention comprises:

determining the inhibition of binding of a ligand to cells which have a polypeptide of the invention on the surface thereof, or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:

(a) incubating a labelled ligand with a whole cell expressing a polypeptide according to the invention on the cell surface, or a cell membrane containing a polypeptide of the invention,

(b) measuring the amount of labelled ligand bound to the whole cell or the cell membrane;

(c) adding a candidate compound to a mixture of labelled ligand and the whole cell or the cell membrane of step (a) and allowing the mixture to attain equilibrium;

(d) measuring the amount of labelled ligand bound to the whole cell or the cell membrane after step (c); and

(e) comparing the difference in the labelled ligand bound in step (b) and (d), such that the compound which causes the reduction in binding in step (d) is considered to be an agonist or antagonist.

The polypeptides may be found to modulate a variety of physiological and pathological processes in a dose-dependent manner in the above-described assays. Thus, the “functional equivalents” of the polypeptides of the invention include polypeptides that exhibit any of the same modulatory activities in the above-described assays in a dose-dependent manner. Although the degree of dose-dependent activity need not be identical to that of the polypeptides of the invention, preferably the “functional equivalents” will exhibit substantially similar dose-dependence in a given activity assay compared to the polypeptides of the invention.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signalling and metabolic pathways in which the particular genes/polypeptides are implicated, generate information regarding the identities of polypeptides with which the studied polypeptides interact and provide clues as to methods by which related genes and proteins are regulated.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X]is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (ie. to prevent infection) or therapeutic (ie. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;
   wherein detection of levels of the hybrid complex in the patient sample that differ from levels of the hybrid complex in the control sample is indicative of disease.

A further aspect of the invention comprises a diagnostic method comprising the steps of:
a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application W095/251116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (ant suitable solid support) and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;

(b) a polypeptide of the present invention; or

(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or disorder or susceptibility to disease or disorder in which alpha-2-macroglobulin-like proteinase inhibitors are implicated. Such diseases and disorders may include reproductive disorders, cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, pancreatitis, arthritis, psoriasis, psoriasis vulgaris, respiratory tract inflammation, asthma, and organ transplant rejection; cardiovascular disorders, including hypertension, oedema, angina, atherosclerosis, thrombosis, sepsis, shock, reperfusion injury, and ischemia, particularly ischemic heart disease; neurological disorders including central nervous system disease, Alzheimer's disease, brain injury, Parkinson's disease, amyotrophic lateral sclerosis, and pain; developmental disorders; metabolic disorders including diabetes mellitus, osteoporosis, and obesity, AIDS, renal disease, particularly idiopathic nephrotic syndrome; lung injury; infections including viral infection, bacterial infection, fungal infection and parasitic infection, particularly Trypanosoma cruzi infection and other pathological conditions. Preferably, the disease is one in which alpha-2-macroglobulin-like proteinase inhibitors are implicated.

Various aspects and embodiments of the present invention will now be described in more detail by way of example, with particular reference to the INSP087 and INSP088 polypeptides.

It will be appreciated that modification of detail may be made without departing from the scope of the invention.

### BRIEF DESCRIPTION OF THE FIGURES

FIG. 1: Top ten results from BLAST against NCBI non-redundant database using INSP087 polypeptide sequence.

FIG. 2: Alignment generated by BLAST between the INSP087 polypeptide sequence and the closest annotated sequence, ovomacroglobulin ovastatin from Gallus gallus.

FIG. 3: Top ten results from BLAST against NCBI non-redundant database using INSP088 polypeptide sequence.

FIG. 4: Alignment generated by BLAST between the INSP088 polypeptide sequence and the closest annotated sequence, alpha-2-macroglobulin precursor from Homo sapiens.

FIG. 5: INSP087 nucleotide sequence with translation showing predicted receptor binding domain (shaded).

FIG. 6: Nucleotide (FIG. 6a) and amino acid (FIG. 6b) alignment of predicted INSP087 receptor binding domain and corresponding region of IMAGE clone 2243344.

FIG. 7: INSP088 prediction nucleotide sequence with translation showing predicted receptor binding domain (shaded).

FIG. 8: Nucleotide (FIG. 8a) and amino acid (FIG. 8b) sequence alignment of predicted INSP088 receptor binding domain and corresponding region of IMAGE clone 4753534.

FIG. 9: Map of pENTR-INSP088-SP-6HIS

FIG. 10: Map of pEAK12d-INSP088-SP-6HIS

FIG. 11: Predicted nucleotide sequence of INSP088 with translation

FIG. 12: Nucleotide sequence with translation of INSP088 PCR product cloned using primers INSP088-CP2 and INSP088-CP3

FIG. 13: Map of pCR4-TOPO-INSP088-CP2/CP3

### EXAMPLES

### Example 1

### INSP087 Protein BLAST Results

The polypeptide sequence given in SEQ ID NO:68, which represents the translation of consecutive exons of INSP087, was used as a BLAST query against the NCBI non-redundant Sequence database.

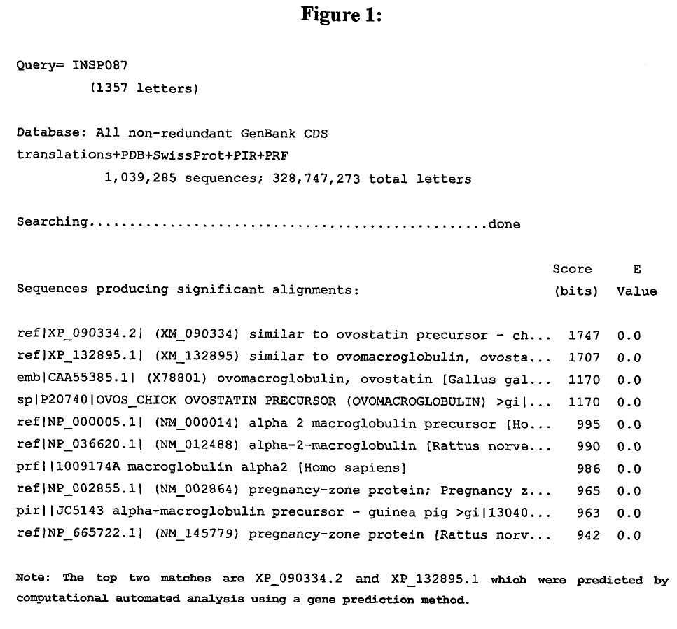

The top ten matches are shown in FIG. 1, all of which are alpha-2-macroglobulin-like proteinase inhibitors.

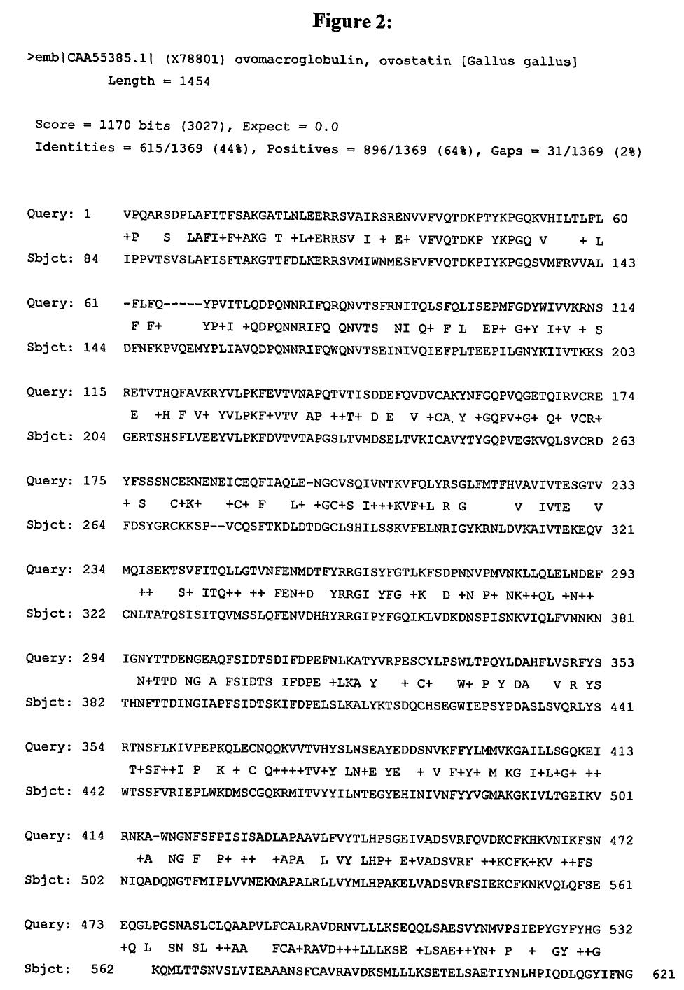

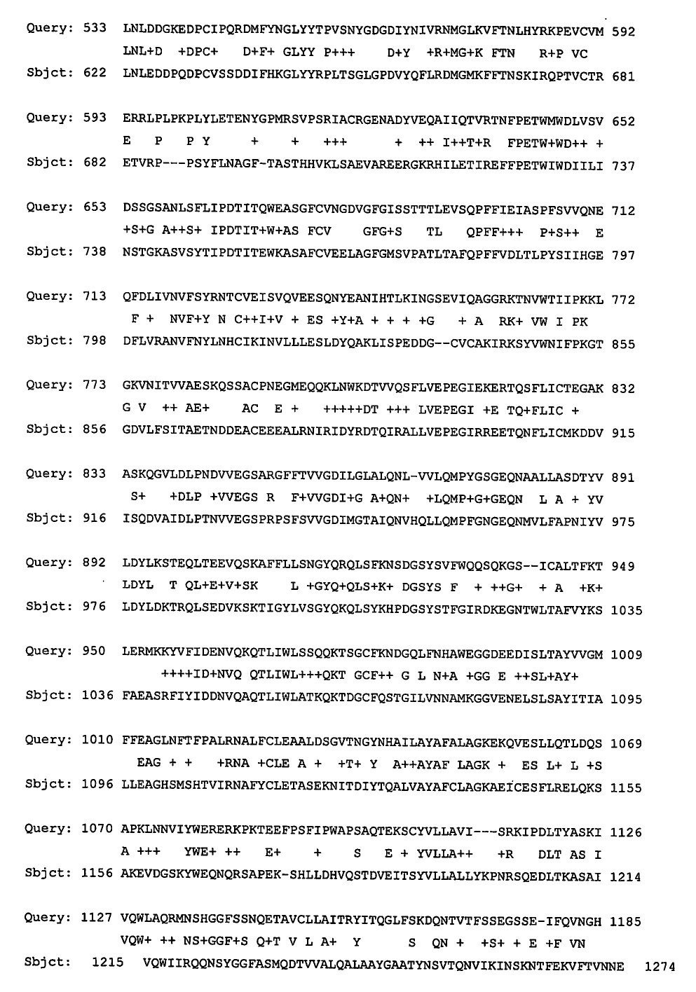

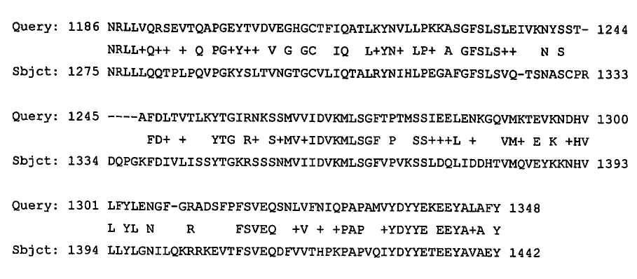

FIG. 2 shows the alignment of the INSP087 query sequence to the sequence of the highest matching known protein, ovomacroglobulin ovastatin (Gallus gallus).

The INSP087 gene has been mapped to a chromosomal location 12p13.31

### Example 2

### INSP088 Protein BLAST Results

The polypeptide sequence given in SEQ ID NO:112, which represents the translation of consecutive exons of INSP088, was used as a BLAST query against the NCBI non-redundant Sequence database.

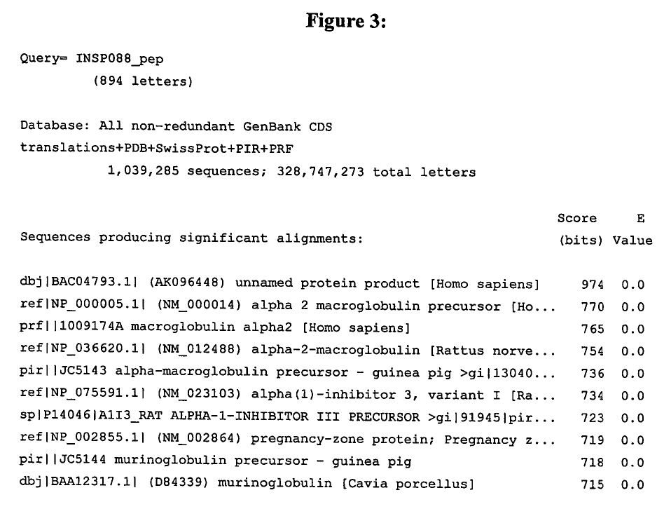

The top ten matches are shown in FIG. 3, all of which are alpha-2-macroglobulin-like proteinase inhibitors.

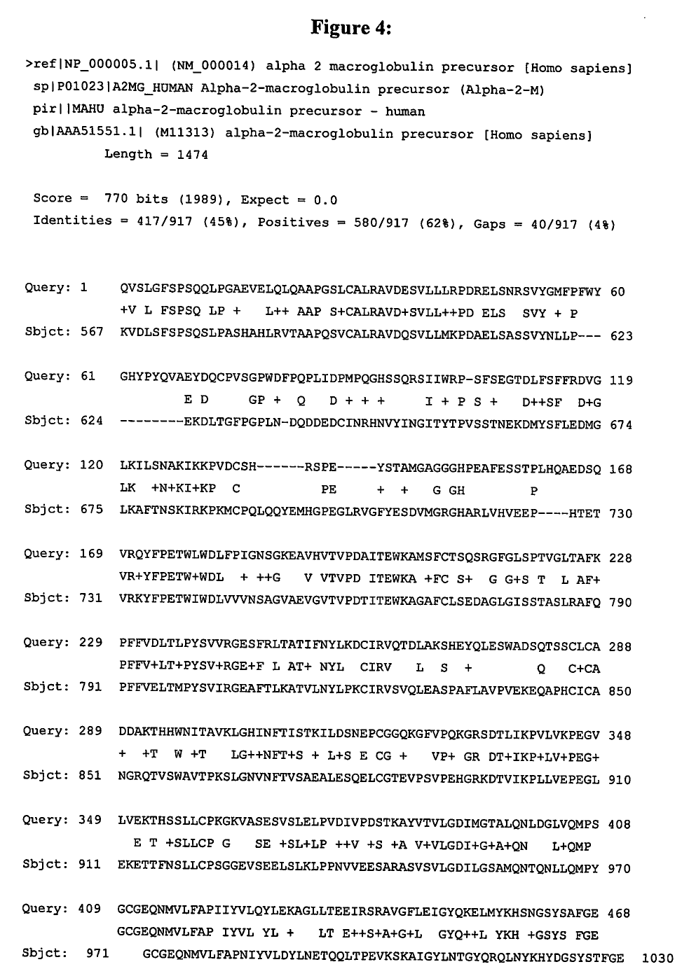

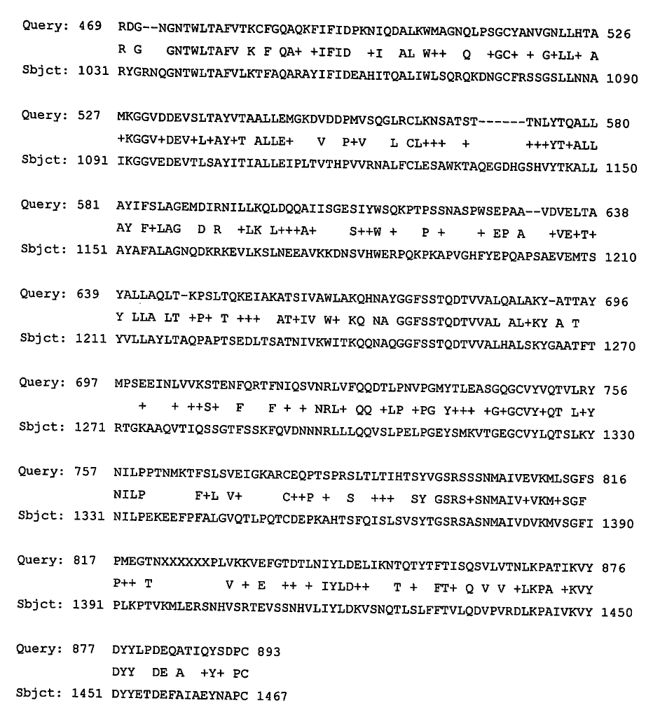

FIG. 4 shows the alignment of the INSP088 query sequence to the sequence of the highest matching known protein, alpha-2-macroglobulin precursor (Homo sapiens).

Expressed sequence tags (ESTs) representing the INSP088 transcript in human originate from the following cDNA libraries: head-neck and brain. This suggests that INSP088 can be cloned from the above tissues and may be associated with diseases of the above tissues. Accordingly, the polypeptides, antibodies and other moieties described herein may have utility in the treating a disease in one of the above tissues.

The INSP088 gene has been mapped to a chromosomal location 12p13.31.

### Example 3

### Cloning of Receptor Binding Domain of INSP087

A 95 amino acid region at the C-terminal end of the INSP087 alpha macroglobulin family member prediction was identified as the putative receptor binding domain of the INSP087 prediction.

Using this region to search public EST databases, several sequences were identified which matched the INSP087 receptor binding domain sequence. One sequence was chosen, GenBank Accession AI655905 corresponding to IMAGE cDNA clone 2243344, and the clone was purchased from ATCC. The insert sequence of the IMAGE clone was sequenced using sequencing primers T7 and T3 (Table 1). The insert sequence was found to contain the predicted INSP087 receptor binding domain. IMAGE clone 2243344 is plasmid database ID 13434.

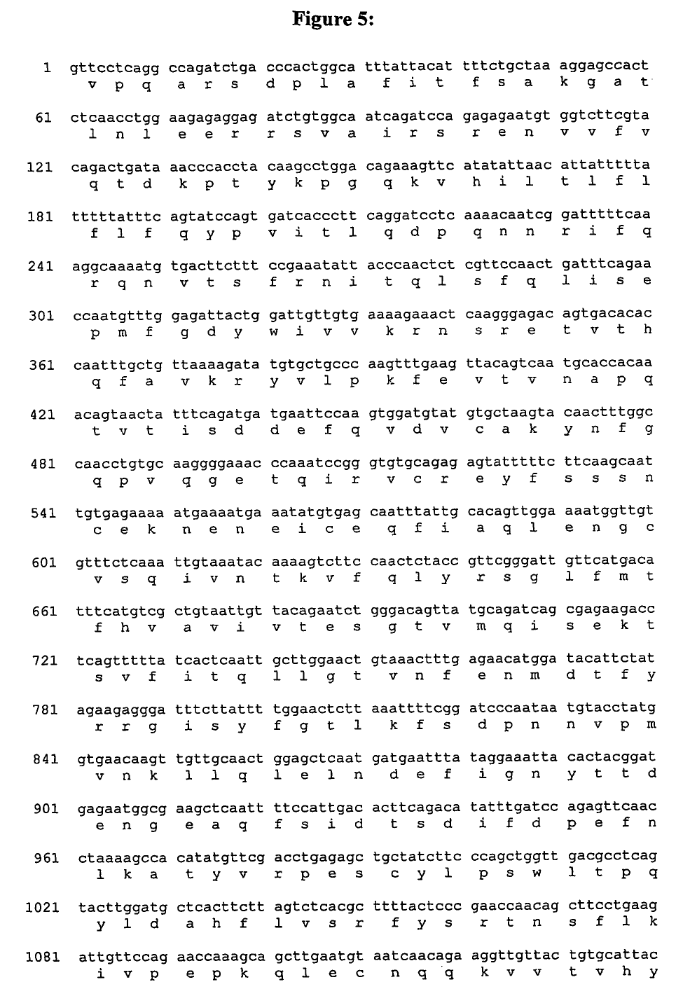

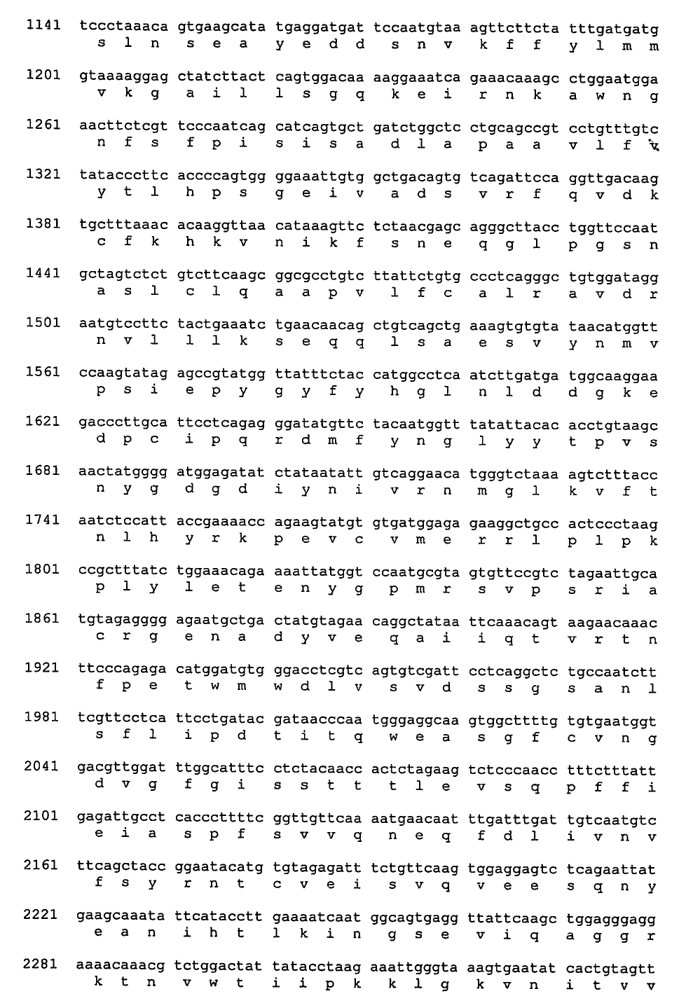

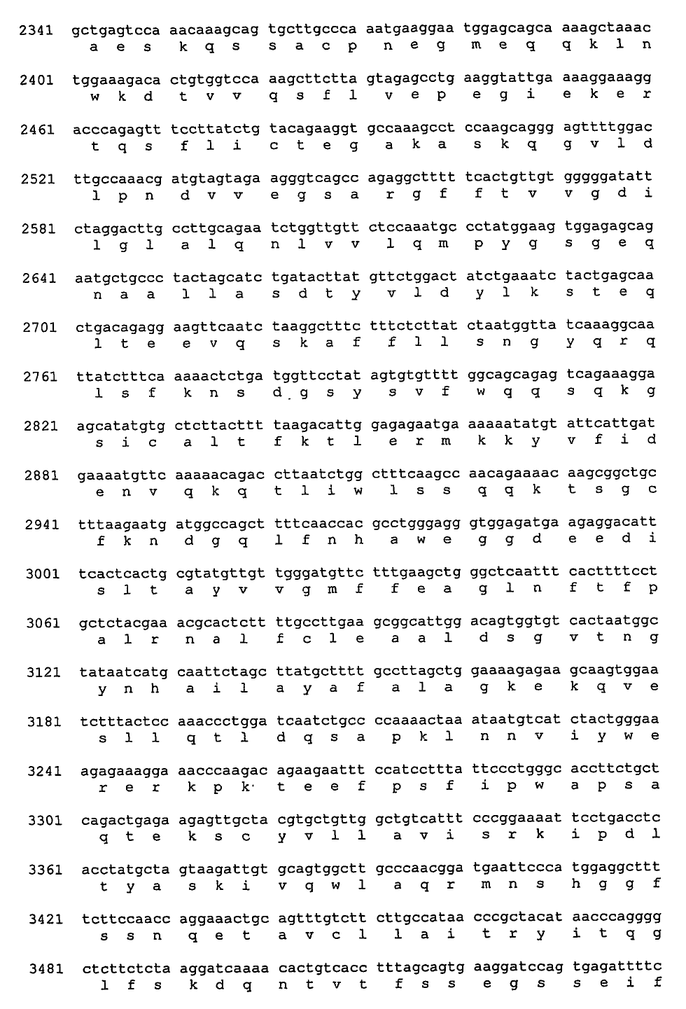

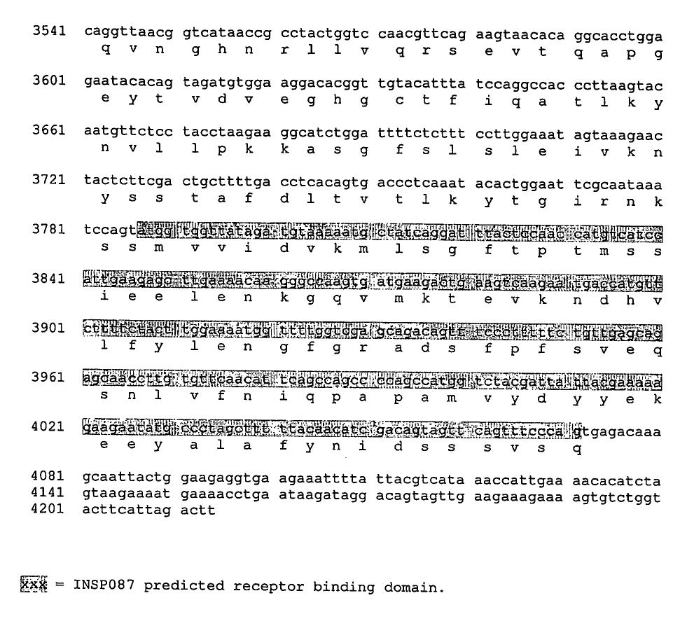

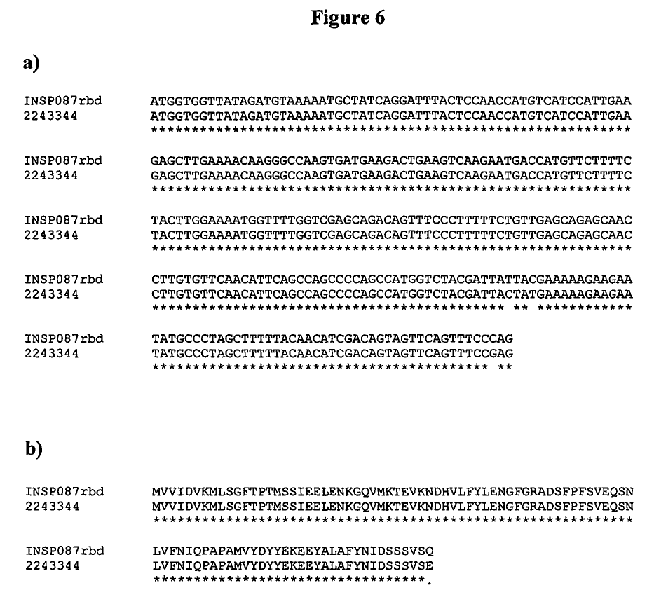

The sequence of the INSP087 prediction with position of the receptor binding domain is shown in FIG. 5. The alignment of the nucleic acid and amino acid sequences of the INSP087 receptor binding domain with the corresponding region of IMAGE clone 2243344 are shown in FIGS. 6a and 6b respectively.

TABLE 1
IMAGE clone 2243344 sequencing primers:

| | |
|---|---|
| Primer | Sequence (5′-3′) |

| | |
|---|---|
| T7 primer | TAA TAC GAC TCA CTA TAG G |
| T3 primer | ATT AAC CCT CAC TAA AGG |

### Example 4

### Cloning of Receptor Binding Domain of INSP088

1. Cloning of INSP088 Receptor Binding Domain

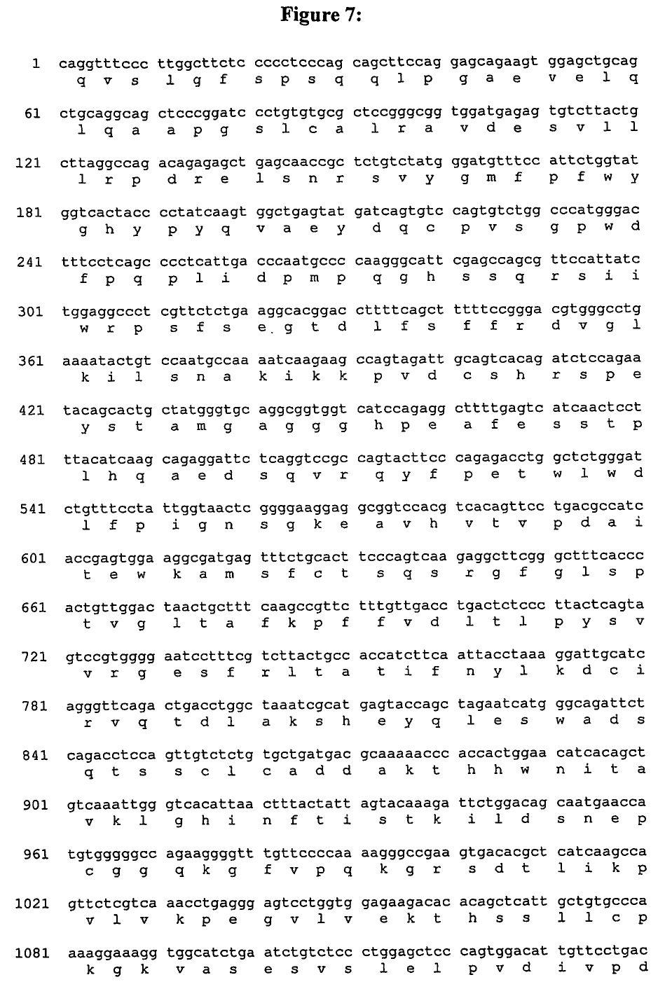

A 92 amino acid region at the C-terminal end of the INSP088 alpha macroglobulin family member prediction was identified as encoding the receptor binding domain. The sequence of the INSP088 prediction with the receptor binding domain highlighted is shown in FIG. 7. Using this domain to search public EST databases, we identified several sequences which matched the INSP088 receptor binding domain sequence. IMAGE cDNA clone 4753534, (GenBank Accession BG680886) was subsequently purchased from ATCC.

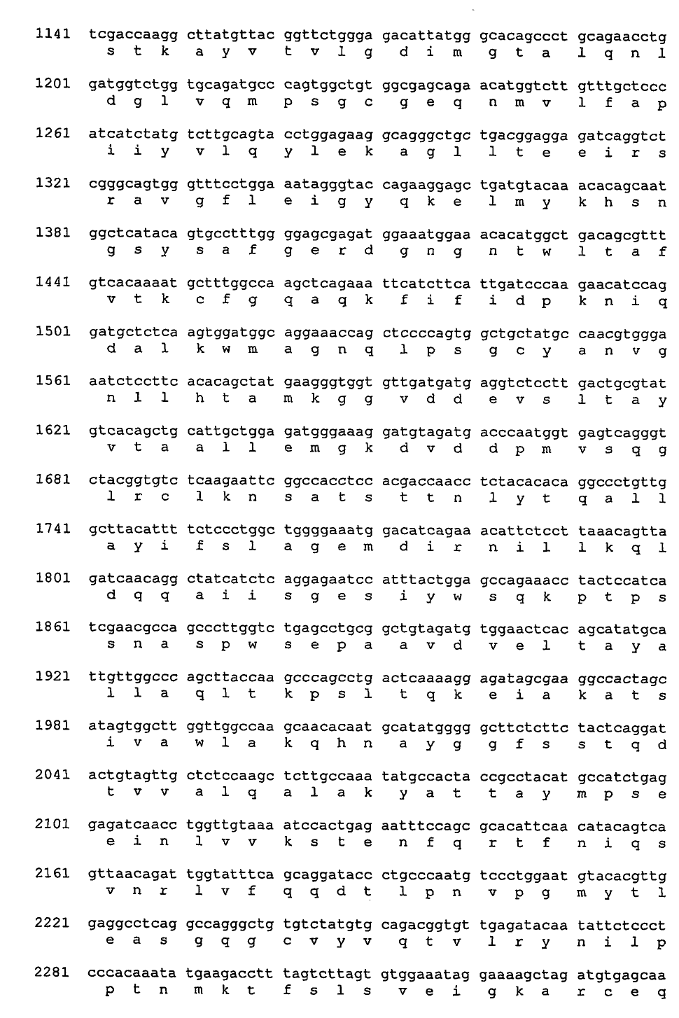

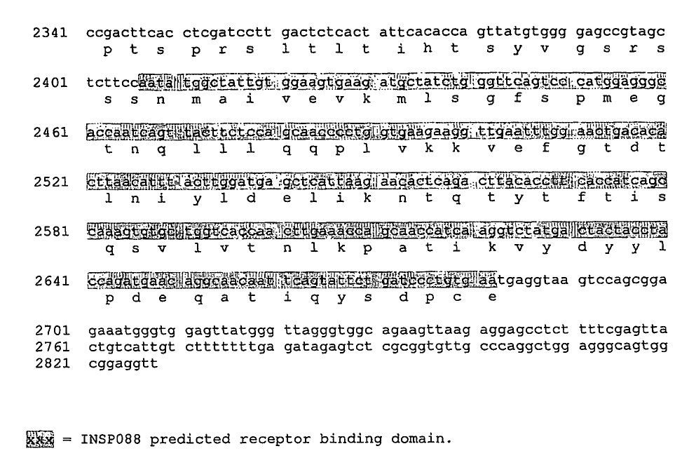

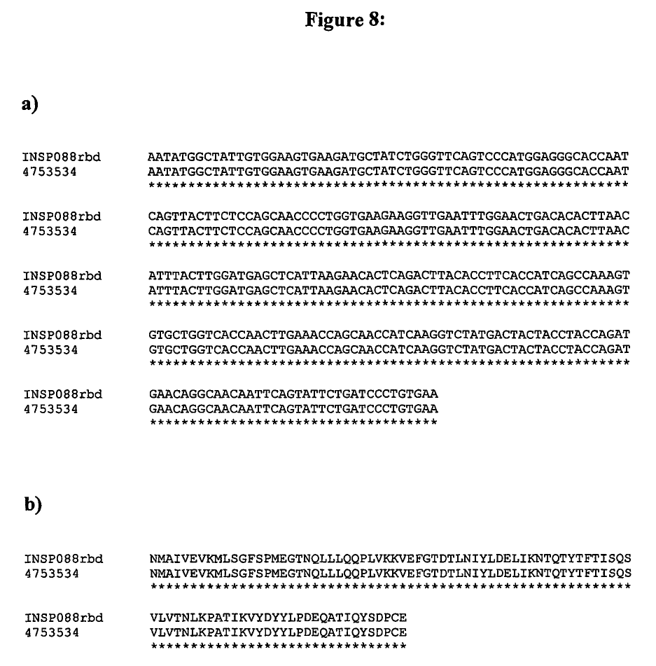

The insert sequence of the IMAGE clone was sequenced using sequencing primers T7 and SP6 (Table 2). The insert sequence was found to contain the predicted INSP088 receptor binding domain. The alignment of the nucleotide and amino acid sequences of the INSP088 receptor binding domain with the corresponding region of IMAGE clone 4753534 is shown in FIGS. 8a and 8b respectively. IMAGE clone 4753534 is plasmid database ID 13684.

2. Subcloning of the INSP088 ORF with a C-Terminal 6HIS Tag and an IL12p40 Signal Sequence into pEAK12d for Expression in HEK293/EBNA Cells

The INSP088 ORF fused to a C-terminal 6HIS tag and a N-terminal signal peptide sequence from IL12p40 was subcloned into pEAK12d mammalian cell using the Gateway cloning methodology.

The first stage of the Gateway cloning process involves a 3 step PCR reaction which generates the ORF of INSP088 flanked at the 5′ end by an attB1 recombination site, a Kozak sequence and the IL12p40 signal sequence, and flanked at the 3′ end by a sequence encoding an in-frame 6 histidine (6HIS) tag, a stop codon and the attB2 recombination site (Gateway compatible cDNA).

The first PCR reaction (in a final volume of 50 μl) contains: 1.5 μl of miniprep DNA prepared from IMAGE clone 4753534 (plasmid ID 13684), 1.5 μl dNTPs (10 mM), 5 μl of 10× Pfx polymerase buffer, 1 μl MgSO4 (50 mM), 0.5 μl each of gene specific primer (100 μM) (INSP088-PCR1F and INSP088-PCR1R) and 0.5 μl Platinum Pfx DNA polymerase (Invitrogen). The PCR reaction was performed using an initial denaturing step of 95° C. for 1 min, followed by 10 cycles of 94° C. for 15 s; 55° C. for 30 s and 68° C. for 1 min 30 s; and a holding cycle of 4° C. PCR products were purified directly from the reaction mixture using the Wizard PCR prep DNA purification system (Promega) according to the manufacturer's instructions.

The second PCR reaction (in a final volume of 50 μl) contained 10 μl purified PCR product, 1.5 μl dNTPs (10 mM), 5 μl of 10× Pfx polymerase buffer, 1 μl MgSO4 (50 mM), 0.5 μl of IL12p40 signal sequence forward primer, IL12P40-SP1 (100 μM), 0.5 μl GCP reverse primer (100 μM) and 0.5 μl of Platinum Pfx DNA polymerase. The conditions for the 2nd PCR reaction were: 95° C. for 1 min, followed by 10 cycles of 94° C. for 15 s; 55° C. for 30 s and 68° C. for 1 min 30 s; and a holding cycle of 4° C. PCR products were purified directly from the reaction mixture using the Wizard PCR prep DNA purification system (Promega) according to the manufacturer's instructions.

The third PCR reaction (in a final volume of 50 μl) contained 10 μl purified PCR product, 1.5 μl dNTPs (10 mM), 5 μl of 10× Pfx polymerase buffer, 1 μl MgSO4 (50 mM), 0.5 μl of Gateway conversion signal peptide forward primer, GCPF-SP (100 μM), 0.5 μl GCP reverse primer (100 μM) and 0.5 μl of Platinum Pfx DNA polymerase. The conditions for the third PCR reaction were: 95° C. for 1 min; 4 cycles of 94° C., 15 sec; 50° C., 30 sec and 68° C. for 1 min 30 s; 25 cycles of 94° C., 15 sec; 55° C., 30 sec and 68° C., 1 min 30 s; followed by a holding cycle of 4° C. PCR products were gel purified using the Wizard PCR prep DNA purification system and eluted in 50 μl of sterile water.

3. Subcloning of Gateway Compatible INSP088 ORF into Gateway Entry Vector pDONR201 and Expression Vector pEAK12d

The second stage of the Gateway cloning process involves subcloning of the Gateway modified PCR product into the Gateway entry vector pDONR201 as follows: 5 μl of purified PCR product is incubated with 1.5 μl pDONR201 vector (0.1 μg/l), 2 μl BP buffer and 1.5 μl of BP clonase enzyme mix (Invitrogen) at RT for 2 h. The reaction was stopped by addition of proteinase K (2 μg) and incubated at 37° C. for a further 10 min.

An aliquot of this reaction (1 μl) was used to transform E. coli DH10B cells by electroporation as follows: a 20 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (1 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (100 μl) were then plated on L-broth (LB) plates containing kanamycin (40 μg/ml) and incubated overnight at 37° C. Transformants were plated on LB-kanamycin plates.

Plasmid mini-prep DNA was prepared from 5 ml cultures from 6 of the resultant colonies using a Qiaprep Turbo 9600 robotic system (Qiagen). Plasmid DNA (200-500 ng) was subjected to DNA sequencing with 21M13 and M13Rev primers using the BigDyeTerminator system (Applied Biosystems cat. no. 4390246) according to the manufacturer's instructions. The primer sequences are shown in Table 2. Sequencing reactions were purified using Dye-Ex columns (Qiagen) or Montage SEQ 96 cleanup plates (Millipore cat. no. LSKS09624) then analyzed on an Applied Biosystems 3700 sequencer.

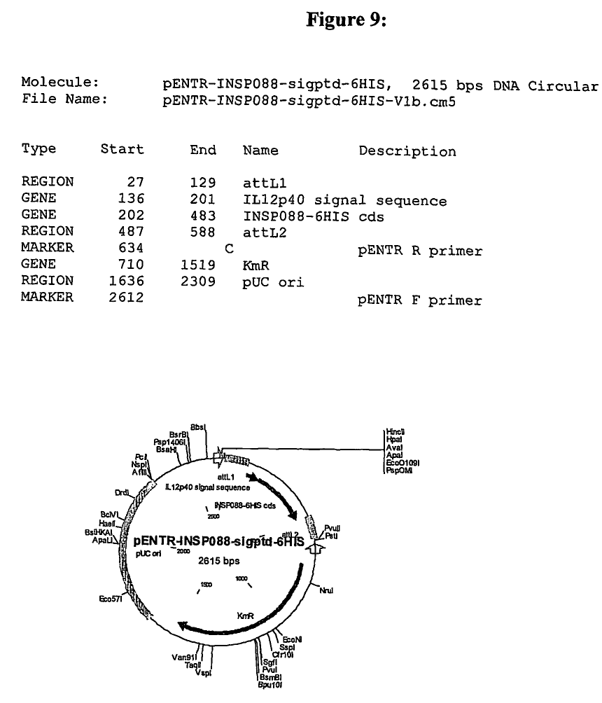

Sequence verified miniprep DNA (pENTR-INSP088-SP-6HIS, plasmid ID 13424, FIG. 9) 3 μl, was then used in a recombination reaction containing 1.5 μl pEAK12d vector (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 μl) was used to transform E. coli DH10B cells by electroporation as follows: a 20 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (1 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (100 μl) were then plated on L-broth (LB) plates containing ampicillin (100 μg/ml) and incubated overnight at 37° C.

Plasmid mini-prep DNA was prepared from 5 ml cultures from 4 of the resultant colonies using a Qiaprep Turbo 9600 robotic system (Qiagen). Plasmid DNA (200-500 ng) was subjected to DNA sequencing with pEAK12d F and pEAK12d R primers as described above.

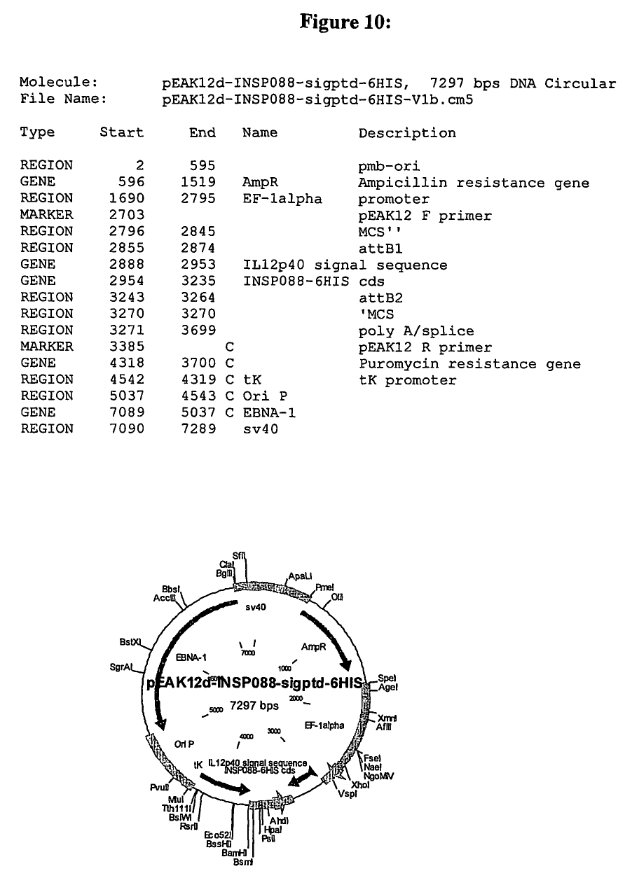

CsCl gradient purified maxi-prep DNA of plasmid pEAK12d-INSP088-SP-6HIS (plasmid ID number 13439) (FIG. 10) was prepared from a 500 ml culture of sequence verified clones (Sambrook J. et al., in Molecular Cloning, Laboratory Manual, 2^{nd} edition, 1989, Cold Spring Harbor Laboratory Press), resuspended at a concentration of 1 μg/μl in sterile water and stored at −20 C.

TABLE 2
INSP088 cloning and sequencing primers

| Primer | Sequence (5′-3′) |
|---|---|
| INSP088- | TTT TCC CTG GTT TTT CTG GCA TCT CCC CTC |
| PCR1F | GTG GCC ATT GTG GAA GTG AA [symbol] ATG CT |
| INSP088- | GTG ATG GTG ATG GTG ACA GGG ATC AGA ATA |
| PCR1R | CTG AA |
| IL12P40- | ATG TGT CAC CAG CAG TTG GTC ATC TCT TGG |
| SP1 | TTT TCC CTG GTT TTT CTG GCA TCT CCC CTC |
| | GTG GCC ATA |
| GCPF-SP | GGGG ACA AGT TTG TAC AAA AAA GCA GGC TTC |
| | GCC ACC ATG TGT CAC CAG CAG TTG |
| GCP | GGG GAC CAC TTT GTA CAA GAA AGC TGG GTT |
| Reverse | TCA ATG GTG ATG GTG ATG GTG |
| T7 primer | TAA TAC GAC TCA CTA TAG G |
| SP6 | ATT TAG GTG ACA CTA TAG |
| primer | |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |

Underlined sequence = Kozak sequence
Bold = Stop codon
Italic sequence = His tag
Bold underlined = IL12p40 signal sequence

### Example 5

### Cloning of Macroglobulin Domain of INSP088

1. Preparation of Human cDNA Templates

First strand cDNA was prepared from a variety of normal human tissue total RNA samples (purchased from Clontech, Stratagene, Ambion, Biochain Institute or prepared in-house) using Superscript II RNAse H⁻ Reverse Transcriptase (Invitrogen) according to the manufacturer's protocol. Oligo (dT)₁₅ primer 1 μl (500 μg/ml, Promega), 2 μg human total RNA, 1 μl 10 mM dNTP Mix (10 mM each of dATP, dGTP, dCTP and dTTP at neutral pH) and sterile distilled water to a final volume of 12 μl were combined in a 1.5 ml Eppendorf tube, heated to 65° C. for 5 min and then chilled on ice.

The contents of the tube were collected by brief centrifugation and 4 μl 5× First-Strand Buffer, 2 μl of 0.1 M DTT, and 1 μl of RnaseOUT Recombinant Ribonuclease Inhibitor (40 units/μl, Invitrogen) were added. The contents of the tube were mixed gently and incubated at 42° C. for 2 min, then 1 μl (200 units) of SuperScript II enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min. The final 21 μl reaction mix was diluted by adding 179 μl sterile water to give a total volume of 200 μl. One μl of each cDNA sample was used as a template for PCR in a 50 μl final reaction volume, which corresponded to approxiumately 20 ng of cDNA.

2. Gene Specific Cloning Primers for PCR

A pair of PCR primers having a length of between 18 and 25 bases were designed for amplifying the predicted macroglobulin domain of the INSP088 prediction. Primers were designed using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (little or no none specific priming).

3. PCR Amplification of INSP088 Macroglobulin Domain from Human cDNA

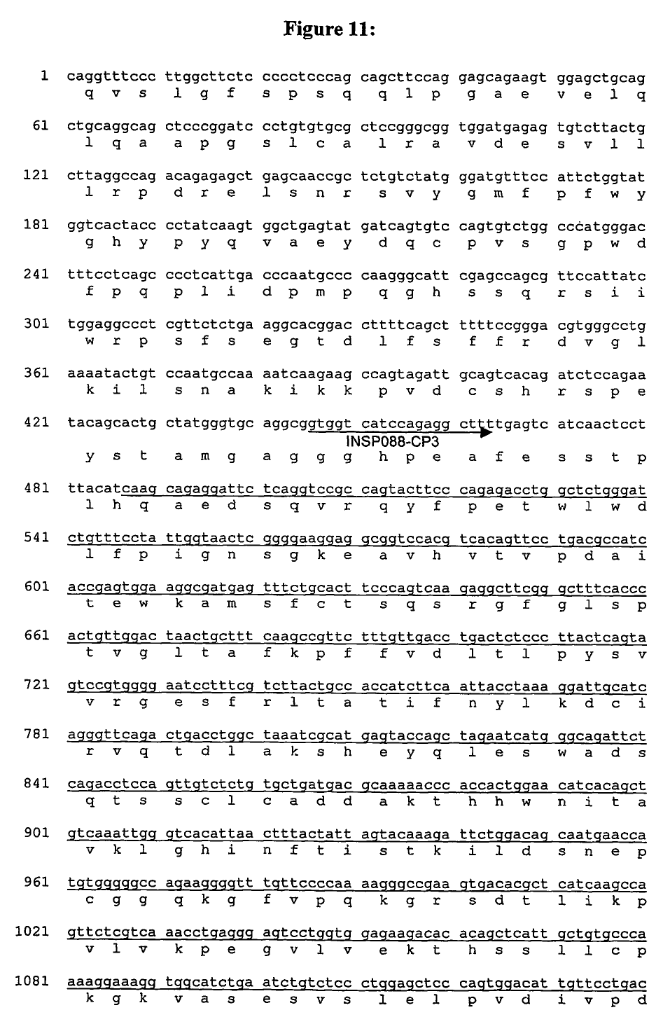

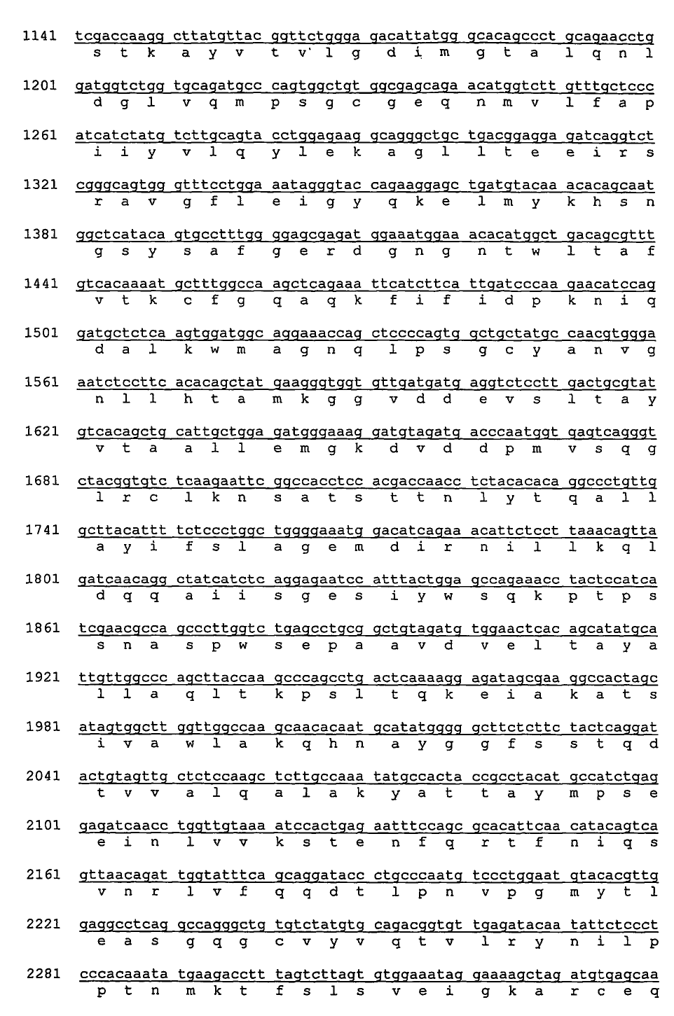

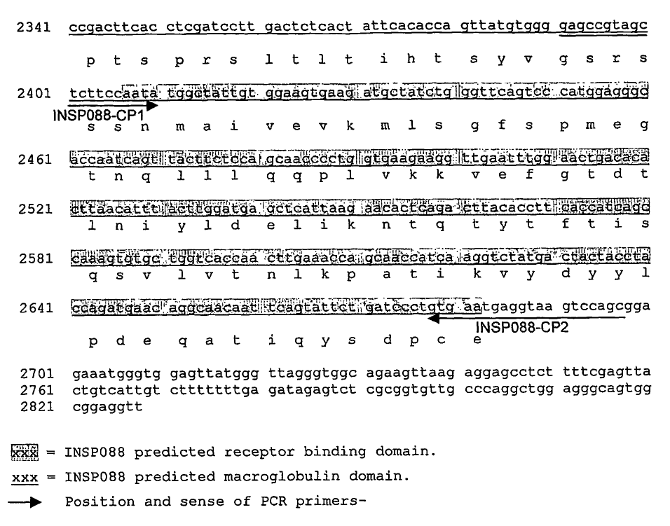

Gene-specific cloning primers INSP088-CP2 and INSP088-CP3 (FIGS. 11 & 12 and Table 3) were designed to amplify a cDNA fragment of 2252 bp covering 446-2697 bp of the coding sequence of the INSP088 prediction.

The primer pair was used with human cDNA samples from brain, testis, colon and skin as templates. The PCR was performed in a final volume of 50 μl containing 1× Easy-A™ reaction buffer, 200 μM dNTPs, 15 pmoles each of cloning primer, 2.5 units of Easy-A™ PCR polymerase (Stratagene) and approximately 20 ng of each cDNA template using an MJ Research DNA Engine, programmed as follows: 95° C., 2 min; 40 cycles of 95° C., 40 sec, 53° C., 30 sec, and 72° C., 3 min; followed by 1 cycle at 72° C. for 7 min and a holding cycle at 4° C. The amplification products were visualized on 0.8% agarose gels in 1× TAE buffer (Invitrogen). PCR products migrating at the predicted molecular mass were purified from the gel using the MinElute Gel Extraction System (QIAGEN). The PCR products were eluted in 10 μl of buffer EB (10 mM Tris.Cl, pH8.5) and subcloned directly.

4. Subcloning of PCR Products

PCR products were subcloned into the topoisomerase I modified cloning vector (pCR4-TOPO) using the TOPO cloning kit purchased from the Invitrogen Corporation using the conditions specified by the manufacturer.

Briefly, 4 μl of gel purified PCR product from the human cDNA amplification was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. The transformation mixture was then plated on L-broth (LB) plates containing ampicillin (100 μg/ml) and incubated overnight at 37° C. Ampicillin resistant colonies containing inserts were identified by colony PCR.

5. Colony PCR

Colonies were inoculated into 50 μl sterile water using a sterile toothpick. A 10 μl aliquot of the inoculum was then subjected to PCR using amplification primers INSP088-CP1 and INSP088-CP2 (Table 3). This pair of primers had been designed to amplify a 307 bp product containing the receptor binding domain of INSP088 located at the 3′ end of the macroglobulin domain. This receptor binding domain had previously been identified in IMAGE clone 4753534 (plasmid ID 13443) which was used as a positive control template for the colony PCR. The PCR was performed in a final volume of 20 μl containing 1× AmpliTaq™ buffer, 200 μM dNTPs, 20 pmoles each of cloning primer, 1 unit of AmpliTaq™ (Perkin Elmer) and 10 μl of the bacterial colony solution using an MJ Research DNA Engine, programmed as follows: 94° C., 2 min; 30 cycles of 94° C., 30 sec, 55° C., 30 sec and 72° C. for 30 sec. Samples were then maintained at 4° C. (holding cycle) before further analysis.

PCR products were analyzed on 1% agarose gels in 1× TAE buffer. Colonies which gave approximately the expected PCR product size (307 bp+187 bp due to the multiple cloning site or MCS) were grown up overnight at 37° C. in 5 ml L-Broth (LB) containing ampicillin (100 μg/ml), with shaking at 220 rpm.

6. Plasmid DNA Preparation and Sequencing

Miniprep plasmid DNA was prepared from 5 ml cultures using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer. Plasmid DNA (200-500 ng) was subjected to DNA sequencing with the T7 primer and T3 primer using the BigDyeTerminator system (Applied Biosystems cat. no. 4390246) according to the manufacturer's instructions. The primer sequences are shown in Table 3. Sequencing reactions were purified using Dye-Ex columns (Qiagen) or Montage SEQ 96 cleanup plates (Millipore cat. no. LSKS09624) then analyzed on an Applied Biosystems 3700 sequencer. Clones which were identified as containing the expected insert after sequencing with the T7 and T3 primers were subjected to further sequencing with gene-specific sequencing primers INSP088-SP1, INSP088-SP2 and INSP088-SP3 (Table 3).

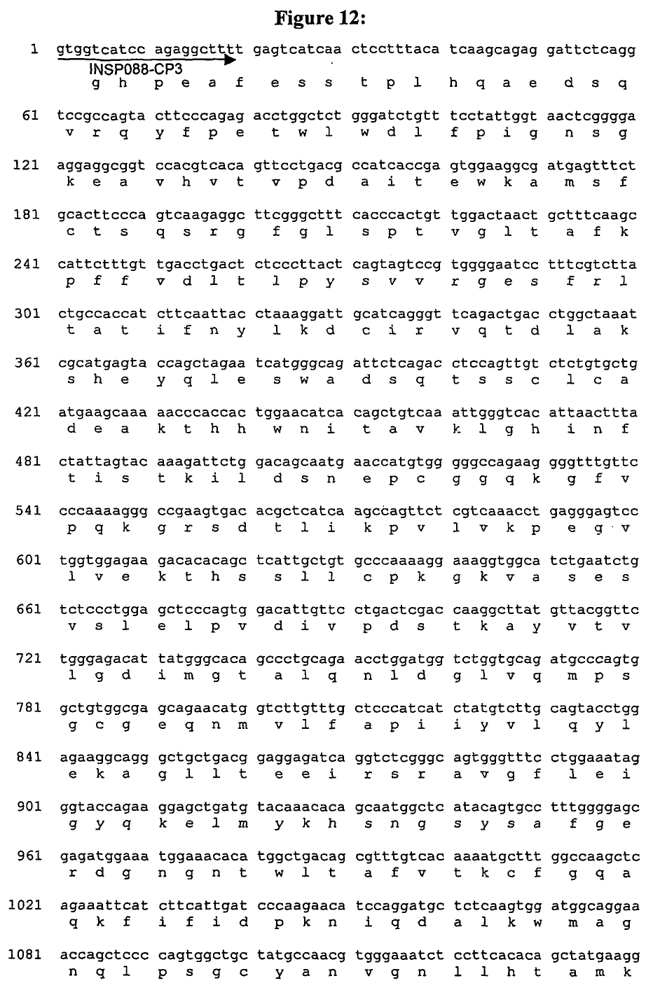

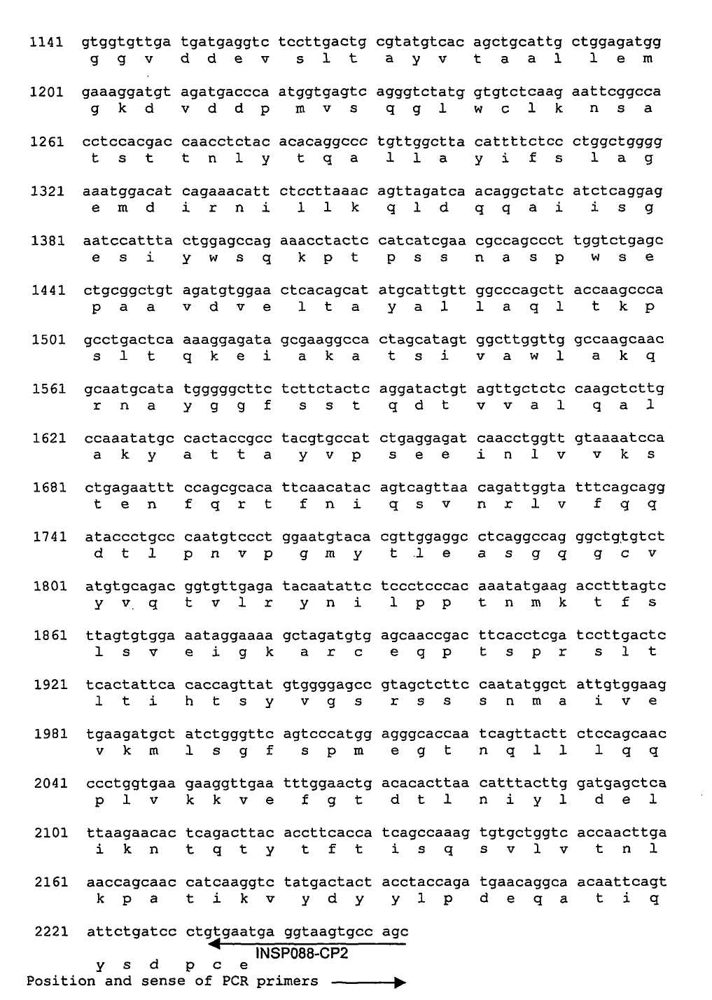

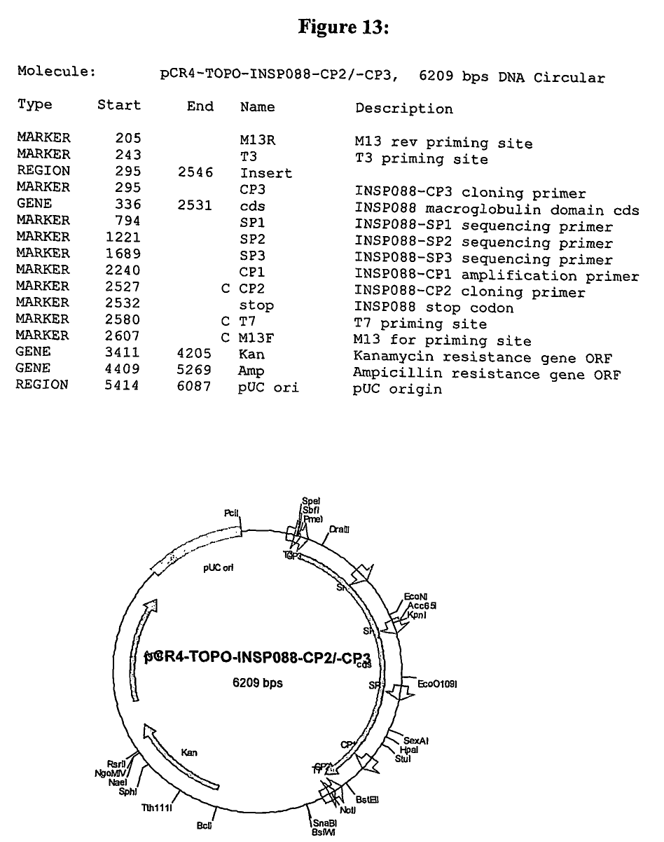

Sequence analysis identified a clone which contained 100% match to the predicted INSP088-CP2/INSP088-CP3 product sequence. The sequence of this cloned cDNA fragment is shown in FIG. 12. The plasmid map of the cloned PCR product pCR4-TOPO-INSP088-CP2/CP3 (plasmid ID 13684) is shown in FIG. 13.

TABLE 3
INSP088 cloning and sequencing primers

| | |
|---|---|
| Primer | Sequence (5′-3′) |

| | |
|---|---|
| INSP088-CP1 | GAG CCG TAG CTC TTC CAA TA |
| INSP088-CP2 | GCT GGA CTT ACC TCA TTC AC |
| INSP088-CP3 | GTG GTC ATC CAG AGG CTT T |
| INSP088-SP1 | GGA CAG CAA TGA ACC ATG TG |
| INSP088-SP2 | CAC AGC AAT GGC TCA TAC AG |
| INSP088-SP3 | AGC CAG AAA CCT ACT CCA TC |
| T7 primer | TAA TAC GAC TCA CTA TAG G |
| T3 primer | ATT AAC CCT CAC TAA AGG |

### Example 6

### Assays for Determining Biological Activity

Differentiation to Adipocyte Assay:

Inhibition of adipocyte differentiation is an in vitro model for reduction of adipose mass believed to be important in reducing insulin resistance in diseases such as diabetes and Polycystic Ovary Syndrome (PCOS). The goal is to identify protein(s) that inhibits differentiation of pre-adipocytes to adipocytes. The 3T3-L1 mouse preadipocyte cell line is induced to differentiate to adipocytes with insulin+IBMX. That differentiation is inhibited by TNF-alpha+cyclohexamide as a positive control.

Tritiated Glucose Uptake (3T3 L1):

The goal of this assay is to identify protein (s) that stimulate glucose uptake as a model for insulin-resistance in adipose during diabetes or PCOS. Adipocytes used are mouse 3T3-L1 preadipocytes that have been differentiated.

Tritiated Glucose Uptake (Primary Human Adipoctes):

The goal of this assay is to identify protein (s) that stimulate glucose uptake as a model for insulin-resistance in adipose during diabetes or PCOS. Primary human adipocytes are used.

Tritiated Glucose Update (Primary Human Skeletal Muscle Cells)

The goal of this assay is to identify protein (s) that stimulate glucose uptake as a model for Insulin-resistance in muscle tissue during diabetes or PCOS. Primary human skeletal muscle cells are differentiated into myotubes and then used in the assay.

INSP087 and INSP088 Sequences (Note: for Amino Acids Encoded by Exon-Exon Junctions, the Amino Acid will be Assigned to the More 5′ Exon.)

| SEQ ID NO: 1 (INSP087 nucleotide sequence exon 1) | |
|---|---|
| 1 | GTTCCTCAGG CCAGATCTGA CCCACTGGCA TTTATTACAT TTTCTGCTAA |
| 51 | AGGAGCCACT CTCAACCTGG AAGAGAGGAG ATCTGTGGCA ATCAGATCCA |
| 101 | GAGAGAATGT GGTCTTCGTA CAGACTGATA AACCCACCTA CAAGCCTGGA |
| 151 | CAGAAAG |
| SEQ ID NO: 2 (INSP087 protein sequence exon 1) | |
| 1 | VPQARSDPLA FITFSAKGAT LNLEERRSVA IRSRENVVFV QTDKPTYKPG |
| 51 | QKV |
| SEQ ID NO: 3 (INSP087 nucleotide sequence exon 2) | |
| 1 | TTCATATATT AACATTATTT TTATTTTTAT TTCAGTATCC AGTGATCACC |
| 51 | CTTCAG |
| SEQ ID NO: 4 (INSP087 protein sequence exon 2) | |
| 1 | HILTLFLFLF QYPVITLQ |
| SEQ ID NO: 5 (INSP087 nucleotide sequence exon 3) | |
| 1 | GATCCTCAAA ACAATCGGAT TTTTCAAAGG CAAAATGTGA CTTCTTTCCG |
| 51 | AAATATTACC CAACTCTCGT TCCAACTGAT TTCAGAACCA ATGTTTGGAG |
| 101 | ATTACTGGAT TGTTGTGAAA AGAAACTCAA GGGAGACAGT GACACACCAA |
| 151 | TTTGCTGTTA AAAGATATG |
| SEQ ID NO: 6 (INSP087 protein sequence exon 3) | |
| 1 | DPQNNRIFQR QNVTSFRNIT QLSFQLISEP MFGDYWIVVK RNSRETVTHQ |
| 51 | FAVKRYV |
| SEQ ID NO: 7 (INSP087 nucleotide sequence exon 4) | |
| 1 | TGCTGCCCAA GTTTGAAGTT ACAGTCAATG CACCACAAAC AGTAACTATT |
| 51 | TCAGATGATG AATTCCAAGT GGATGTATGT GCTAA |
| SEQ ID NO: 8 (INSP087 protein sequence exon 4) | |
| 1 | LPKFEVTVNA PQTVTISDDE FQVDVCAK |
| SEQ ID NO: 9 (INSP087 nucleotide sequence exon 5) | |
| 1 | GTACAACTTT GGCCAACCTG TGCAAGGGGA AACCCAAATC CGGGTGTGCA |
| 51 | GAGAGTATTT TTCTTCAAGC AATTGTGAGA AAAATGAAAA TGAAATATGT |
| 101 | GAGCAATTTA TTGCACAG |
| SEQ ID NO: 10 (INSP087 protein sequence exon 5) | |
| 1 | YNFGQPVQGE TQIRVCREYF SSSNCEKNEN EICEQFIAQ |
| SEQ ID NO: 11 (INSP087 nucleotide sequence exon 6) | |
| 1 | TTGGAAAATG GTTGTGTTTC TCAAATTGTA AATACAAAAG TCTTCCAACT |
| 51 | CTACCGTTCG GGATTGTTCA TGACATTTCA TGTCGCTGTA ATTGTTACAG |
| 101 | AATCTGGGAC AG |
| SEQ ID NO: 12 (INSP087 protein sequence exon 6) | |
| 1 | LENGCVSQIV NTKVFQLYRS GLFMTFHVAV IVTESGTV |
| SEQ ID NO: 13 (INSP087 nucleotide sequence exon 7) | |
| 1 | TTATGCAGAT CAGCGAGAAG ACCTCAGTTT TTATCACTCA ATTGCTTGGA |
| 51 | ACTGTAAACT TTGAGAACAT GGATACATTC TATAGAAGAG GGATTTCTTA |
| 101 | TTTTGGAACT |
| SEQ ID NO: 14 (INSP087 protein sequence exon 7) | |
| 1 | MQISEKTSVF ITQLLGTVNF ENMDTFYRRG ISYFGT |
| SEQ ID NO: 15 (INSP087 nucleotide sequence exon 8) | |
| 1 | CTTAAATTTT CGGATCCCAA TAATGTACCT ATGGTGAACA AGTTGTTGCA |
| 51 | ACTGGAGCTC AATGATGAAT TTATAGGAAA TTACACTACG GATGAGAATG |
| 101 | GCGAAGCTCA ATTTTCCATT GACACTTCAG ACATATTTGA TCCAGAGTTC |
| 151 | AACCTAAAA |
| SEQ ID NO: 16 (INSP087 protein sequence exon 8) | |
| 1 | LKFSDPNNVP MVNKLLQLEL NDEFIGNYTT DENGEAQFSI DTSDIFDPEF |
| 51 | NLK |
| SEQ ID NO: 17 (INSP087 nucleotide sequence exon 9) | |
| 1 | GCCACATATG TTCGACCTGA GAGCTGCTAT CTTCCCAGCT GGTTGACGCC |
| 51 | TCAGTACTTG GATGCTCACT TCTTAGTCTC ACGCTTTTAC TCCCGAACCA |
| 101 | ACAGCTTCCT GAAGATTGTT CCAGAACCAA AGCAGCTTGA ATGTAATCAA |
| 151 | CAGAAGGTTG TTACTGTGCA TTACTCCCTA AACAGTGAAG CATATGAGGA |
| 201 | TGATTCCAAT GTAAAGTTCT TCTATTTG |
| SEQ ID NO: 18 (INSP087 protein sequence exon 9) | |
| 1 | ATYVRPESCY LPSWLTPQYL DAHFLVSRFY SRTNSFLKIV PEPKQLECNQ |
| 51 | QKVVTVHYSL NSEAYEDDSN VKFFYL |
| SEQ ID NO: 19 (INSP087 nucleotide sequence exon 10) | |
| 1 | ATGATGGTAA AAGGAGCTAT CTTACTCAGT GGACAAAAGG AAATCAGAAA |
| 51 | CAAAG |
| SEQ ID NO: 20 (INSP087 protein sequence exon 10) | |
| 1 | MMVKGAILLS GQKEIRNKA |
| SEQ ID NO: 21 (INSP087 nucleotide sequence exon 11) | |
| 1 | CCTGGAATGG AAACTTCTCG TTCCCAATCA GCATCAGTGC TGATCTGGCT |
| 51 | CCTGCAGCCG TCCTGTTTGT CTATACCCTT CACCCCAGTG GGGAAATTGT |
| 101 | GGCTGACAGT GTCAGATTCC AGGTTGACAA GTGCTTTAAA CACAAG |
| SEQ ID NO: 22 (INSP087 protein sequence exon 11) | |
| 1 | WNGNFSFPIS ISADLAPAAV LFVYTLHPSG EIVADSVRFQ VDKCFKHK |
| SEQ ID NO: 23 (INSP087 nucleotide sequence exon 12) | |
| 1 | GTTAACATAA AGTTCTCTAA CGAGCAGGGC TTACCTGGTT CCAATGCTAG |
| 51 | TCTCTGTCTT CAAGCGGCGC CTGTCTTATT CTGTGCCCTC AGGGCTGTGG |
| 101 | ATAGGAATGT CCTTCTACTG AAATCTGAAC AACAGCTGTC AGCTGAAAGT |
| SEQ ID NO: 24 (INSP087 protein sequence exon 12) | |
| 1 | VNIKFSNEQG LPGSNASLCL QAAPVLFCAL RAVDRNVLLL KSEQQLSAES |
| SEQ ID NO: 25 (INSP087 nucleotide sequence exon 13) | |
| 1 | GTGTATAACA TGGTTCCAAG TATAGAGCCG TATGGTTATT TCTACCATGG |
| 51 | CCTCAATCTT GATGATGGCA AGGAAGACCC TTGCATTCCT CAGAGGGATA |
| 101 | TGTTCTACAA TGGTTTATAT TACACACCTG TAAGCAACTA TGGGGATGGA |
| 151 | GATATCTATA ATATTGTCAG G |
| SEQ ID NO: 26 (INSP087 protein sequence exon 13) | |
| 1 | VYNMVPSIEP YGYFYHGLNL DDGKEDPCIP QRDMFYNGLY YTPVSNYGDG |
| 51 | DIYNIVR |
| SEQ ID NO: 27 (INSP087 nucleotide sequence exon 14) | |
| 1 | AACATGGGTC TAAAAGTCTT TACCAATCTC CATTACCGAA AACCAGAAGT |
| 51 | ATGTGTGATG GAGAGAAGGC TGCCACTCCC TAAGCCGCTT TATCTGGAAA |
| 101 | CAGAAAATTA TGGTCCAATG CGTAGTGTTC CGTCTAGAAT TGCATGTAG |
| SEQ ID NO: 28 (INSP087 protein sequence exon 14) | |
| 1 | NMGLKVFTNL HYRKPEVCVM ERRLPLPKPL YLETENYGPM RSVPSRIACR |
| SEQ ID NO: 29 (INSP087 nucleotide sequence exon 15) | |
| 1 | AGGGGAGAAT GCTGACTATG TAGAACAGGC TATAATTCAA ACAGTAAGAA |
| 51 | CAAACTTCCC AGAGACATGG ATGTGGGACC TCGTCAGTGT CGA |
| SEQ ID NO: 30 (INSP087 protein sequence exon 15) | |
| 1 | GENADYVEQA IIQTVRTNFP ETWMWDLVSV D |
| SEQ ID NO: 31 (INSP087 nucleotide sequence exon 16) | |
| 1 | TTCCTCAGGC TCTGCCAATC TTTCGTTCCT CATTCCTGAT ACGATAACCC |
| 51 | AATGGGAGGC AAGTGGCTTT TGTGTGAATG GTGACGTTGG ATTTGGCATT |
| 101 | TCCTCTACAA CCACTCTAGA AGTCTCCCAA CCTTTCTTTA TTGAGATTGC |
| 151 | CTCACCCTTT TCGGTTGTTC AAAATGAACA ATTTGATTTG ATTGTCAATG |
| 201 | TCTTCAGCTA CCGGAATACA TGTGTAGAG |
| SEQ ID NO: 32 (INSP087 protein sequence exon 16) | |
| 1 | SSGSANLSFL IPDTITQWEA SGFCVNGDVG FGISSTTTLE VSQPFFIEIA |
| 51 | SPFSVVQNEQ FDLIVNVFSY RNTCVE |
| SEQ ID NO: 33 (INSP087 nucleotide sequence exon 17) | |
| 1 | ATTTCTGTTC AAGTGGAGGA GTCTCAGAAT TATGAAGCAA ATATTCATAC |
| 51 | CTTGAAAATC AATGGCAGTG AGGTTATTCA AGCTGGAGGG AGGAAAACAA |
| 101 | ACGTCTGGAC TATTATACCT AAGAAATTGG |
| SEQ ID NO: 34 (INSP087 protein sequence exon 17) | |
| 1 | ISVQVEESQN YEANIHTLKI NGSEVIQAGG RKTNVWTIIP KKLG |
| SEQ ID NO: 35 (INSP087 nucleotide sequence exon 18) | |
| 1 | GTAAAGTGAA TATCACTGTA GTTGCTGAGT CCAAACAAAG CAGTGCTTGC |
| 51 | CCAAATGAAG GAATGGAGCA GCAAAAGCTA AACTGGAAAG ACACTGTGGT |
| 101 | CCAAAGCTTC TTAGTAGAG |
| SEQ ID NO: 36 (INSP087 protein sequence exon 18) | |
| 1 | KVNITVVAES KQSSACPNEG MEQQKLNWKD TVVQSFLVE |
| SEQ ID NO: 37 (INSP087 nucleotide sequence exon 19) | |
| 1 | CCTGAAGGTA TTGAAAAGGA AAGGACCCAG AGTTTCCTTA TCTGTACAGA |
| 51 | AG |
| SEQ ID NO: 38 (INSP087 protein sequence exon 19) | |
| 1 | PEGIEKERTQ SFLICTEG |
| SEQ ID NO: 39 (INSP087 nucleotide sequence exon 20) | |
| 1 | GTGCCAAAGC CTCCAAGCAG GGAGTTTTGG ACTTGCCAAA CGATGTAGTA |
| 51 | GAAGGGTCAG CCAGAGGCTT TTTCACTGTT GTGG |
| SEQ ID NO: 40 (INSP087 protein sequence exon 20) | |
| 1 | AKASKQGVLD LPNDVVEGSA RGFFTVVG |
| SEQ ID NO: 41 (INSP087 nucleotide sequence exon 21) | |
| 1 | GGGATATTCT AGGACTTGCC TTGCAGAATC TGGTTGTTCT CCAAATGCCC |
| 51 | TATGGAAGTG GAGAGCAGAA TGCTGCCCTA CTAGCATCTG ATACTTATGT |
| 101 | TCTGGACTAT CTGAAATCTA CTGAGCAACT GACAGAGGAA GTTCAATCTA |
| 151 | AGGCTTTCTT TCTCTTATCT AATG |
| SEQ ID NO: 42 (INSP087 protein sequence exon 21) | |
| 1 | DILGLALQNL VVLQMPYGSG EQNAALLASD TYVLDYLKST EQLTEEVQSK |
| 51 | AFFLLSNG |
| SEQ ID NO: 43 (INSP087 nucleotide sequence exon 22) | |
| 1 | GTTATCAAAG GCAATTATCT TTCAAAAACT CTGATGGTTC CTATAGTGTG |
| 51 | TTTTGGCAGC AGAGTCAGAA AGGAAGCATA TG |
| SEQ ID NO: 44 (INSP087 protein sequence exon 22) | |
| 1 | YQRQLSFKNS DGSYSVFWQQ SQKGSIC |
| SEQ ID NO: 45 (INSP087 nucleotide sequence exon 23) | |
| 1 | TGCTCTTACT TTTAAGACAT TGGAGAGAAT GAAAAAATAT GTATTCATTG |
| 51 | ATGAAAATGT TCAAAAACAG ACCTTAATCT GGCTTTCAAG CCAACAGAAA |
| 101 | ACAAGCGGCT GCTTTAAGAA TGATGGCCAG CTTTTCAACC ACGCCTGGGA |
| 151 | G |
| SEQ ID NO: 46 (INSP087 protein sequence exon 23) | |
| 1 | ALTFKTLERM KKYVFIDENV QKQTLIWLSS QQKTSGCFKN DGQLFNHAWE |
| SEQ ID NO: 47 (INSP087 nucleotide sequence exon 24) | |
| 1 | GGTGGAGATG AAGAGGACAT TTCACTCACT GCGTATGTTG TTGGGATGTT |
| 51 | CTTTGAAGCT GGGCTCAATT TCACT |
| SEQ ID NO: 48 (INSP087 protein sequence exon 24) | |
| 1 | GGDEEDISLT AYVVGMFFEA GLNFT |
| SEQ ID NO: 49 (INSP087 nucleotide sequence exon 25) | |
| 1 | TTTCCTGCTC TACGAAACGC ACTCTTTTGC CTTGAAGCGG CATTGGACAG |
| 51 | TGGTGTCACT AATGGCTATA ATCATGCAAT TCTAGCTTAT GCTTTTGCCT |
| 101 | TAGCTGGAAA AGAGAAGCAA GTGGAATCTT TACTCCAAAC CCTGGATCAA |
| 151 | TCTGCCCCAA AACTAA |
| SEQ ID NO: 50 (INSP087 protein sequence exon 25) | |
| 1 | FPALRNALFC LEAALDSGVT NGYNHAILAY AFALAGKEKQ VESLLQTLDQ |
| 51 | SAPKLN |
| SEQ ID NO: 51 (INSP087 nucleotide sequence exon 26) | |
| 1 | ATAATGTCAT CTACTGGGAA AGAGAAAGGA AACCCAAGAC AGAAGAATTT |
| 51 | CCATCCTTTA TTCCCTGGGC ACCTTCTGCT CAGACTGAGA AGAGTTGCTA |
| 101 | CGTGCTGTTG GCTGTCATTT CCCGGAAAAT TCCTGACCTC ACCTATGCTA |
| 151 | GTAAGATTGT GCAGTGGCTT GCCCAACGGA TGAATTCCCA TGGAGGCTTT |
| 201 | TCTTCCAACC AG |
| SEQ ID NO: 52 (INSP087 protein sequence exon 26) | |
| 1 | NVIYWERERK PKTEEFPSFI PWAPSAQTEK SCYVLLAVIS RKIPDLTYAS |
| 51 | KIVQWLAQRM NSHGGFSSNQ |
| SEQ ID NO: 53 (INSP087 nucleotide sequence exon 27) | |
| 1 | GAAACTGCAG TTTGTCTTCT TGCCATAACC CGCTACATAA CCC |
| SEQ ID NO: 54 (INSP087 protein sequence exon 27) | |
| 1 | ETAVCLLAIT RYITQ |
| SEQ ID NO: 55 (INSP087 nucleotide sequence exon 28) | |
| 1 | AGGGGCTCTT CTCTAAGGAT CAAAACACTG TCACCTTTAG CAGTGAAGGA |
| 51 | TCCAGTGAGA TTTTCCAGGT TAACGGTCAT AACCGCCTAC TGGTCCAACG |
| 101 | TTCAGAAGTA ACACAGGCAC CTGGAGAATA CACAGTAGAT GTGGAAGGAC |
| 151 | ACGGTTGTAC ATTTATCCAG |
| SEQ ID NO: 56 (INSP087 protein sequence exon 28) | |
| 1 | GLFSKDQNTV TFSSEGSSEI FQVNGHNRLL VQRSEVTQAP GEYTVDVEGH |
| 51 | GCTFIQ |
| SEQ ID NO: 57 (INSP087 nucleotide sequence exon 29) | |
| 1 | GCCACCCTTA AGTACAATGT TCTCCTACCT AAGAAGGCAT CTGGATTTTC |
| 51 | TCTTTCCTTG GAAATAGTAA AGAACTACTC TTCGACTGCT TTTGACCTCA |
| 101 | CAGTGACCCT CAA |
| SEQ ID NO: 58 (INSP087 protein sequence exon 29) | |
| 1 | ATLKYNVLLP KKASGFSLSL EIVKNYSSTA FDLTVTLK |
| SEQ ID NO: 59 (INSP087 nucleotide sequence exon 30) | |
| 1 | ATACACTGGA ATTCGCAATA AATCCAGTAT GGTGGTTATA GATGTAAAAA |
| 51 | TGCTATCAGG ATTTACTCCA ACCATGTCAT CCATTGAAGA G |
| SEQ ID NO: 60 (INSP087 protein sequence exon 30) | |
| 1 | YTGIRNKSSM VVIDVKMLSG FTPTMSSIEE |
| SEQ ID NO: 61 (INSP087 nucleotide sequence exon 31) | |
| 1 | CTTGAAAACA AGGGCCAAGT GATGAAGACT GAAGTCAAGA ATGACCATGT |
| 51 | TCTTTTCTAC TTGGAAAAT |
| SEQ ID NO: 62 (INSP087 protein sequence exon 31) | |
| 1 | LENKGQVMKT EVKNDHVLFY LEN |
| SEQ ID NO: 63 (INSP087 nucleotide sequence exon 32) | |
| 1 | GGTTTTGGTC GAGCAGACAG TTTCCCTTTT TCTGTTGAGC AGAGCAACCT |
| 51 | TGTGTTCAAC ATTCAGCCAG CCCCAGCCAT GGTCTACGAT TATTACGAAA |
| 101 | AAG |
| SEQ ID NO: 64 (INSP087 protein sequence exon 32) | |
| 1 | GFGRADSFPF SVEQSNLVFN IQPAPAMVYD YYEKE |
| SEQ ID NO: 65 (INSP087 nucleotide sequence exon 33) | |
| 1 | AAGAATATGC CCTAGCTTTT TACAACATCG ACAGTAGTTC AGTTTCCCAG |
| 51 | TGA |
| SEQ ID NO: 66 (INSP087 protein sequence exon 33) | |
| 1 | EYALAFYNID SSSVSQ |
| SEQ ID NO: 67 (INSP087 nucleotide sequence) | |
| 1 | GTTCCTCAGG CCAGATCTGA CCCACTGGCA TTTATTACAT TTTCTGCTAA |
| 51 | AGGAGCCACT CTCAACCTGG AAGAGAGGAG ATCTGTGGCA ATCAGATCCA |
| 101 | GAGAGAATGT GGTCTTCGTA CAGACTGATA AACCCACCTA CAAGCCTGGA |
| 151 | CAGAAAGTTC ATATATTAAC ATTATTTTTA TTTTTATTTC AGTATCCAGT |
| 201 | GATCACCCTT CAGGATCCTC AAAACAATCG GATTTTTCAA AGGCAAAATG |
| 251 | TGACTTCTTT CCGAAATATT ACCCAACTCT CGTTCCAACT GATTTCAGAA |
| 301 | CCAATGTTTG GAGATTACTG GATTGTTGTG AAAAGAAACT CAAGGGAGAC |
| 351 | AGTGACACAC CAATTTGCTG TTAAAAGATA TGTGCTGCCC AAGTTTGAAG |
| 401 | TTACAGTCAA TGCACCACAA ACAGTAACTA TTTCAGATGA TGAATTCCAA |
| 451 | GTGGATGTAT GTGCTAAGTA CAACTTTGGC CAACCTGTGC AAGGGGAAAC |
| 501 | CCAAATCCGG GTGTGCAGAG AGTATTTTTC TTCAAGCAAT TGTGAGAAAA |
| 551 | ATGAAAATGA AATATGTGAG CAATTTATTG CACAGTTGGA AAATGGTTGT |
| 601 | GTTTCTCAAA TTGTAAATAC AAAAGTCTTC CAACTCTACC GTTCGGGATT |
| 651 | GTTCATGACA TTTCATGTCG CTGTAATTGT TACAGAATCT GGGACAGTTA |
| 701 | TGCAGATCAG CGAGAAGACC TCAGTTTTTA TCACTCAATT GCTTGGAACT |
| 751 | GTAAACTTTG AGAACATGGA TACATTCTAT AGAAGAGGGA TTTCTTATTT |
| 801 | TGGAACTCTT AAATTTTCGG ATCCCAATAA TGTACCTATG GTGAACAAGT |
| 851 | TGTTGCAACT GGAGCTCAAT GATGAATTTA TAGGAAATTA CACTACGGAT |
| 901 | GAGAATGGCG AAGCTCAATT TTCCATTGAC ACTTCAGACA TATTTGATCC |
| 951 | AGAGTTCAAC CTAAAAGCCA CATATGTTCG ACCTGAGAGC TGCTATCTTC |
| 1001 | CCAGCTGGTT GACGCCTCAG TACTTGGATG CTCACTTCTT AGTCTCACGC |
| 1051 | TTTTACTCCC GAACCAACAG CTTCCTGAAG ATTGTTCCAG AACCAAAGCA |
| 1101 | GCTTGAATGT AATCAACAGA AGGTTGTTAC TGTGCATTAC TCCCTAAACA |
| 1151 | GTGAAGCATA TGAGGATGAT TCCAATGTAA AGTTCTTCTA TTTGATGATG |
| 1201 | GTAAAAGGAG CTATCTTACT CAGTGGACAA AAGGAAATCA GAAACAAAGC |
| 1251 | CTGGAATGGA AACTTCTCGT TCCCAATCAG CATCAGTGCT GATCTGGCTC |
| 1301 | CTGCAGCCGT CCTGTTTGTC TATACCCTTC ACCCCAGTGG GGAAATTGTG |
| 1351 | GCTGACAGTG TCAGATTCCA GGTTGACAAG TGCTTTAAAC ACAAGGTTAA |
| 1401 | CATAAAGTTC TCTAACGAGC AGGGCTTACC TGGTTCCAAT GCTAGTCTCT |
| 1451 | GTCTTCAAGC GGCGCCTGTC TTATTCTGTG CCCTCAGGGC TGTGGATAGG |
| 1501 | AATGTCCTTC TACTGAAATC TGAACAACAG CTGTCAGCTG AAAGTGTGTA |
| 1551 | TAACATGGTT CCAAGTATAG AGCCGTATGG TTATTTCTAC CATGGCCTCA |
| 1601 | ATCTTGATGA TGGCAAGGAA GACCCTTGCA TTCCTCAGAG GGATATGTTC |
| 1651 | TACAATGGTT TATATTACAC ACCTGTAAGC AACTATGGGG ATGGAGATAT |
| 1701 | CTATAATATT GTCAGGAACA TGGGTCTAAA AGTCTTTACC AATCTCCATT |
| 1751 | ACCGAAAACC AGAAGTATGT GTGATGGAGA GAAGGCTGCC ACTCCCTAAG |
| 1801 | CCGCTTTATC TGGAAACAGA AAATTATGGT CCAATGCGTA GTGTTCCGTC |
| 1851 | TAGAATTGCA TGTAGAGGGG AGAATGCTGA CTATGTAGAA CAGGCTATAA |
| 1901 | TTCAAACAGT AAGAACAAAC TTCCCAGAGA CATGGATGTG GGACCTCGTC |
| 1951 | AGTGTCGATT CCTCAGGCTC TGCCAATCTT TCGTTCCTCA TTCCTGATAC |
| 2001 | GATAACCCAA TGGGAGGCAA GTGGCTTTTG TGTGAATGGT GACGTTGGAT |
| 2051 | TTGGCATTTC CTCTACAACC ACTCTAGAAG TCTCCCAACC TTTCTTTATT |
| 2101 | GAGATTGCCT CACCCTTTTC GGTTGTTCAA AATGAACAAT TTGATTTGAT |
| 2151 | TGTCAATGTC TTCAGCTACC GGAATACATG TGTAGAGATT TCTGTTCAAG |
| 2201 | TGGAGGAGTC TCAGAATTAT GAAGCAAATA TTCATACCTT GAAAATCAAT |
| 2251 | GGCAGTGAGG TTATTCAAGC TGGAGGGAGG AAAACAAACG TCTGGACTAT |
| 2301 | TATACCTAAG AAATTGGGTA AAGTGAATAT CACTGTAGTT GCTGAGTCCA |
| 2351 | AACAAAGCAG TGCTTGCCCA AATGAAGGAA TGGAGCAGCA AAAGCTAAAC |
| 2401 | TGGAAAGACA CTGTGGTCCA AAGCTTCTTA GTAGAGCCTG AAGGTATTGA |
| 2451 | AAAGGAAAGG ACCCAGAGTT TCCTTATCTG TACAGAAGGT GCCAAAGCCT |
| 2501 | CCAAGCAGGG AGTTTTGGAC TTGCCAAACG ATGTAGTAGA AGGGTCAGCC |
| 2551 | AGAGGCTTTT TCACTGTTGT GGGGGATATT CTAGGACTTG CCTTGCAGAA |
| 2601 | TCTGGTTGTT CTCCAAATGC CCTATGGAAG TGGAGAGCAG AATGCTGCCC |
| 2651 | TACTAGCATC TGATACTTAT GTTCTGGACT ATCTGAAATC TACTGAGCAA |
| 2701 | CTGACAGAGG AAGTTCAATC TAAGGCTTTC TTTCTCTTAT CTAATGGTTA |
| 2751 | TCAAAGGCAA TTATCTTTCA AAAACTCTGA TGGTTCCTAT AGTGTGTTTT |
| 2801 | GGCAGCAGAG TCAGAAAGGA AGCATATGTG CTCTTACTTT TAAGACATTG |
| 2851 | GAGAGAATGA AAAAATATGT ATTCATTGAT GAAAATGTTC AAAAACAGAC |
| 2901 | CTTAATCTGG CTTTCAAGCC AACAGAAAAC AAGCGGCTGC TTTAAGAATG |
| 2951 | ATGGCCAGCT TTTCAACCAC GCCTGGGAGG GTGGAGATGA AGAGGACATT |
| 3001 | TCACTCACTG CGTATGTTGT TGGGATGTTC TTTGAAGCTG GGCTCAATTT |
| 3051 | CACTTTTCCT GCTCTACGAA ACGCACTCTT TTGCCTTGAA GCGGCATTGG |
| 3101 | ACAGTGGTGT CACTAATGGC TATAATCATG CAATTCTAGC TTATGCTTTT |
| 3151 | GCCTTAGCTG GAAAAGAGAA GCAAGTGGAA TCTTTACTCC AAACCCTGGA |
| 3201 | TCAATCTGCC CCAAAACTAA ATAATGTCAT CTACTGGGAA AGAGAAAGGA |
| 3251 | AACCCAAGAC AGAAGAATTT CCATCCTTTA TTCCCTGGGC ACCTTCTGCT |
| 3301 | CAGACTGAGA AGAGTTGCTA CGTGCTGTTG GCTGTCATTT CCCGGAAAAT |
| 3351 | TCCTGACCTC ACCTATGCTA GTAAGATTGT GCAGTGGCTT GCCCAACGGA |
| 3401 | TGAATTCCCA TGGAGGCTTT TCTTCCAACC AGGAAACTGC AGTTTGTCTT |
| 3451 | CTTGCCATAA CCCGCTACAT AACCCAGGGG CTCTTCTCTA AGGATCAAAA |
| 3501 | CACTGTCACC TTTAGCAGTG AAGGATCCAG TGAGATTTTC CAGGTTAACG |
| 3551 | GTCATAACCG CCTACTGGTC CAACGTTCAG AAGTAACACA GGCACCTGGA |
| 3601 | GAATACACAG TAGATGTGGA AGGACACGGT TGTACATTTA TCCAGGCCAC |
| 3651 | CCTTAAGTAC AATGTTCTCC TACCTAAGAA GGCATCTGGA TTTTCTCTTT |
| 3701 | CCTTGGAAAT AGTAAAGAAC TACTCTTCGA CTGCTTTTGA CCTCACAGTG |
| 3751 | ACCCTCAAAT ACACTGGAAT TCGCAATAAA TCCAGTATGG TGGTTATAGA |
| 3801 | TGTAAAAATG CTATCAGGAT TTACTCCAAC CATGTCATCC ATTGAAGAGC |
| 3851 | TTGAAAACAA GGGCCAAGTG ATGAAGACTG AAGTCAAGAA TGACCATGTT |
| 3901 | CTTTTCTACT TGGAAAATGG TTTTGGTCGA GCAGACAGTT TCCCTTTTTC |
| 3951 | TGTTGAGCAG AGCAACCTTG TGTTCAACAT TCAGCCAGCC CCAGCCATGG |
| 4001 | TCTACGATTA TTACGAAAAA GAAGAATATG CCCTAGCTTT TTACAACATC |
| 4051 | GACAGTAGTT CAGTTTCCCA GTGA |
| SEQ ID NO: 68 (INSP087 protein sequence) | |
| 1 | VPQARSDPLA FITFSAKGAT LNLEERRSVA IRSRENVVFV QTDKPTYKPG |
| 51 | QKVHILTLFL FLFQYPVITL QDPQNNRIFQ RQNVTSFRNI TQLSFQLISE |
| 101 | PMFGDYWIVV KRNSRETVTH QFAVKRYVLP KFEVTVNAPQ TVTISDDEFQ |
| 151 | VDVCAKYNFG QPVQGETQIR VCREYFSSSN CEKNENEICE QFIAQLENGC |
| 201 | VSQIVNTKVF QLYRSGLFMT FHVAVIVTES GTVMQISEKT SVFITQLLGT |
| 251 | VNFENMDTFY RRGISYFGTL KFSDPNNVPM VNKLLQLELN DEFIGNYTTD |
| 301 | ENGEAQFSID TSDIFDPEFN LKATYVRPES CYLPSWLTPQ YLDAHFLVSR |
| 351 | FYSRTNSFLK IVPEPKQLEC NQQKVVTVHY SLNSEAYEDD SNVKFFYLMM |
| 401 | VKGAILLSGQ KEIRNKAWNG NFSFPISISA DLAPAAVLFV YTLHPSGEIV |
| 451 | ADSVRFQVDK CFKHKVNIKF SNEQGLPGSN ASLCLQAAPV LFCALRAVDR |
| 501 | NVLLLKSEQQ LSAESVYNMV PSIEPYGYFY HGLNLDDGKE DPCIPQRDMF |
| 551 | YNGLYYTPVS NYGDGDIYNI VRNMGLKVFT NLHYRKPEVC VMERRLPLPK |
| 601 | PLYLETENYG PMRSVPSRIA CRGENADYVE QAIIQTVRTN FPETWMWDLV |
| 651 | SVDSSGSANL SFLIPDTITQ WEASGFCVNG DVGFGISSTT TLEVSQPFFI |
| 701 | EIASPFSVVQ NEQFDLIVNV FSYRNTCVEI SVQVEESQNY EANIHTLKIN |
| 751 | GSEVIQAGGR KTNVWTIIPK KLGKVNITVV AESKQSSACP NEGMEQQKLN |
| 801 | WKDTVVQSFL VEPEGIEKER TQSFLICTEG AKASKQGVLD LPNDVVEGSA |
| 851 | RGFFTVVGDI LGLALQNLVV LQMPYGSGEQ NAALLASDTY VLDYLKSTEQ |
| 901 | LTEEVQSKAF FLLSNGYQRQ LSFKNSDGSY SVFWQQSQKG SICALTFKTL |
| 951 | ERMKKYVFID ENVQKQTLIW LSSQQKTSGC FKNDGQLFNH AWEGGDEEDI |
| 1001 | SLTAYVVGMF FEAGLNFTFP ALRNALFCLE AALDSGVTNG YNHAILAYAF |
| 1051 | ALAGKEKQVE SLLQTLDQSA PKLNNVIYWE RERKPKTEEF PSFIPWAPSA |
| 1101 | QTEKSCYVLL AVISRKIPDL TYASKIVQWL AQRMNSHGGF SSNQETAVCL |
| 1151 | LAITRYITQG LFSKDQNTVT FSSEGSSEIF QVNGHNRLLV QRSEVTQAPG |
| 1201 | EYTVDVEGHG CTFIQATLKY NVLLPKKASG FSLSLEIVKN YSSTAFDLTV |
| 1251 | TLKYTGIRNK SSMVVIDVKM LSGFTPTMSS IEELENKGQV MKTEVKNDHV |
| 1301 | LFYLENGFGR ADSFPFSVEQ SNLVFNIQPA PAMVYDYYEK EEYALAFYNI |
| 1351 | DSSSVSQ |
| SEQ ID NO: 69 (INSP088 nucleotide sequence exon 1) | |
| 1 | CAGGTTTCCC TTGGCTTCTC CCCCTCCCAG CAGCTTCCAG GAGCAGAAGT |
| 51 | GGAGCTGCAG CTGCAGGCAG CTCCCGGATC CCTGTGTGCG CTCCGGGCGG |
| 101 | TGGATGAGAG TGTCTTACTG CTTAGGCCAG ACAGAGAGCT GAGCAACCGC |
| 151 | TCT |
| SEQ ID NO: 70 (INSP088 protein sequence exon 1) | |
| 1 | QVSLGFSPSQ QLPGAEVELQ LQAAPGSLCA LRAVDESVLL LRPDRELSNR |
| 51 | S |
| SEQ ID NO: 71 (INSP088 nucleotide sequence exon 2) | |
| 1 | GTCTATGGGA TGTTTCCATT CTGGTATGGT CACTACCCCT ATCAAGTGGC |
| 51 | TGAGTATGAT CAGTGTCCAG TGTCTGGCCC ATGGGACTTT CCTCAGCCCC |
| 101 | TCATTGACCC AATGCCCCAA GGGCATTCGA GCCAGCGTTC CATTATCTGG |
| 151 | AGGCCCTCGT TCTCTGAAGG CACGGACCTT TTCAGCTTTT TCCGG |
| SEQ ID NO: 72 (INSP088 protein sequence exon 2) | |
| 1 | VYGMFPFWYG HYPYQVAEYD QCPVSGPWDF PQPLIDPMPQ GHSSQRSIIW |
| 51 | RPSFSEGTDL FSFFR |
| SEQ ID NO: 73 (INSP088 nucleotide sequence exon 3) | |
| 1 | GACGTGGGCC TGAAAATACT GTCCAATGCC AAAATCAAGA AGCCAGTAGA |
| 51 | TTGCAGTCAC AGATCTCCAG AATACAGCAC TGCTATGGGT G |
| SEQ ID NO: 74 (INSP088 protein sequence exon 3) | |
| 1 | DVGLKILSNA KIKKPVDCSH RSPEYSTAMG A |
| SEQ ID NO: 75 (INSP088 nucleotide sequence exon 4) | |
| 1 | CAGGCGGTGG TCATCCAGAG GCTTTTGAGT CATCAACTCC TTTACATCAA |
| 51 | GCAGAGGATT CTCAGGTCCG CCAGTACTTC CCAGAGACCT GGCTCTGGGA |
| 101 | TCTGTTTCCT ATTGG |
| SEQ ID NO: 76 (INSP088 protein sequence exon 4) | |
| 1 | GGGHPEAFES STPLHQAEDS QVRQYFPETW LWDLFPIG |
| SEQ ID NO: 77 (INSP088 nucleotide sequence exon 5) | |
| 1 | TAACTCGGGG AAGGAGGCGG TCCACGTCAC AGTTCCTGAC GCCATCACCG |
| 51 | AGTGGAAGGC GATGAGTTTC TGCACTTCCC AGTCAAGAGG CTTCGGGCTT |
| 101 | TCACCCACTG TTGGACTAAC TGCTTTCAAG CCGTTCTTTG TTGACCTGAC |
| 151 | TCTCCCTTAC TCAGTAGTCC GTGGGGAATC CTTTCGTCTT ACTGCCACCA |
| 201 | TCTTCAATTA CCTAAAGGAT TGCATCAGG |
| SEQ ID NO: 78 (INSP088 protein sequence exon 5) | |
| 1 | NSGKEAVHVT VPDAITEWKA MSFCTSQSRG FGLSPTVGLT AFKPFFVDLT |
| 51 | LPYSVVRGES FRLTATIFNY LKDCIR |
| SEQ ID NO: 79 (INSP088 nucleotide sequence exon 6) | |
| 1 | GTTCAGACTG ACCTGGCTAA ATCGCATGAG TACCAGCTAG AATCATGGGC |
| 51 | AGATTCTCAG ACCTCCAGTT GTCTCTGTGC TGATGACGCA AAAACCCACC |
| 101 | ACTGGAACAT CACAGCTGTC AAATTGG |
| SEQ ID NO: 80 (INSP088 protein sequence exon 6) | |
| 1 | VQTDLAKSHE YQLESWADSQ TSSCLCADDA KTHHWNITAV KLG |
| SEQ ID NO: 81 (INSP088 nucleotide sequence exon 7) | |
| 1 | GTCACATTAA CTTTACTATT AGTACAAAGA TTCTGGACAG CAATGAACCA |
| 51 | TGTGGGGGCC AGAAGGGGTT TGTTCCCCAA AAGGGCCGAA GTGACACGCT |
| 101 | CATCAAGCCA GTTCTCGTCA AA |
| SEQ ID NO: 82 (INSP088 protein sequence exon 7) | |
| 1 | HINFTISTKI LDSNEPCGGQ KGFVPQKGRS DTLIKPVLVK |
| SEQ ID NO: 83 (INSP088 nucleotide sequence exon 8) | |
| 1 | CCTGAGGGAG TCCTGGTGGA GAAGACACAC AGCTCATTGC TGTGCCCAAA |
| 51 | AG |
| SEQ ID NO: 84 (INSP088 protein sequence exon 8) | |
| 1 | PEGVLVEKTH SSLLCPKG |
| SEQ ID NO: 85 (INSP088 nucleotide sequence exon 9) | |
| 1 | GAAAGGTGGC ATCTGAATCT GTCTCCCTGG AGCTCCCAGT GGACATTGTT |
| 51 | CCTGACTCGA CCAAGGCTTA TGTTACGGTT CTGG |
| SEQ ID NO: 86 (INSP088 protein sequence exon 9) | |
| 1 | KVASESVSLE LPVDIVPDST KAYVTVLG |
| SEQ ID NO: 87 (1INSP088 nucleotide sequence exon 10) | |
| 1 | GAGACATTAT GGGCACAGCC CTGCAGAACC TGGATGGTCT GGTGCAGATG |
| 51 | CCCAGTGGCT GTGGCGAGCA GAACATGGTC TTGTTTGCTC CCATCATCTA |
| 101 | TGTCTTGCAG TACCTGGAGA AGGCAGGGCT GCTGACGGAG GAGATCAGGT |
| 151 | CTCGGGCAGT GGGTTTCCTG GAAATAG |
| SEQ ID NO: 88 (INSP088 protein sequence exon 10) | |
| 1 | DIMGTALQNL DGLVQMPSGC GEQNMVLFAP IIYVLQYLEK AGLLTEEIRS |
| 51 | RAVGFLEIG |
| SEQ ID NO: 89 (INSP088 nucleotide sequence exon 11) | |
| 1 | GGTACCAGAA GGAGCTGATG TACAAACACA GCAATGGCTC ATACAGTGCC |
| 51 | TTTGGGGAGC GAGATGGAAA TGGAAACACA TG |
| SEQ ID NO: 90 (INSP088 protein sequence exon 11) | |
| 1 | YQKELMYKHS NGSYSAFGER DGNGNTW |
| SEQ ID NO: 91 (INSP088 nucleotide sequence exon 12) | |
| 1 | GCTGACAGCG TTTGTCACAA AATGCTTTGG CCAAGCTCAG AAATTCATCT |
| 51 | TCATTGATCC CAAGAACATC CAGGATGCTC TCAAGTGGAT GGCAGGAAAC |
| 101 | CAGCTCCCCA GTGGCTGCTA TGCCAACGTG GGAAATCTCC TTCACACAGC |
| 151 | TATGAAG |
| SEQ ID NO: 92 (INSP088 protein sequence exon 12) | |
| 1 | LTAFVTKCFG QAQKFIFIDP KNIQDALKWM AGNQLPSGCY ANVGNLLHTA |
| 51 | MK |
| SEQ ID NO: 93 (INSP088 nucleotide sequence exon 13) | |
| 1 | GGTGGTGTTG ATGATGAGGT CTCCTTGACT GCGTATGTCA CAGCTGCATT |
| 51 | GCTGGAGATG GGAAAGGATG TAGAT |
| SEQ ID NO: 94 (INSP088 protein sequence exon 13) | |
| 1 | GGVDDEVSLT AYVTAALLEM GKDVD |
| SEQ ID NO: 95 (INSP088 nucleotide sequence exon 14) | |
| 1 | GACCCAATGG TGAGTCAGGG TCTACGGTGT CTCAAGAATT CGGCCACCTC |
| 51 | CACGACCAAC CTCTACACAC AGGCCCTGTT GGCTTACATT TTCTCCCTGG |
| 101 | CTGGGGAAAT GGACATCAGA AACATTCTCC TTAAACAGTT AGATCAACAG |
| 151 | GCTATCATCT CAG |
| SEQ ID NO: 96 (INSP088 protein sequence exon 14) | |
| 1 | DPMVSQGLRC LKNSATSTTN LYTQALLAYI FSLAGEMDIR NILLKQLDQQ |
| 51 | AIISG |
| SEQ ID NO: 97 (INSP088 nucleotide sequence exon 15) | |
| 1 | GAGAATCCAT TTACTGGAGC CAGAAACCTA CTCCATCATC GAACGCCAGC |
| 51 | CCTTGGTCTG AGCCTGCGGC TGTAGATGTG GAACTCACAG CATATGCATT |
| 101 | GTTGGCCCAG CTTACCAAGC CCAGCCTGAC TCAAAAGGAG ATAGCGAAGG |
| 151 | CCACTAGCAT AGTGGCTTGG TTGGCCAAGC AACACAATGC ATATGGGGGC |
| 201 | TTCTCTTCTA CTCAG |
| SEQ ID NO: 98 (INSP088 protein sequence exon 15) | |
| 1 | ESIYWSQKPT PSSNASPWSE PAAVDVELTA YALLAQLTKP SLTQKEIAKA |
| 51 | TSIVAWLAKQ HNAYGGFSST Q |
| SEQ ID NO: 99 (INSP088 nucleotide sequence exon 16) | |
| 1 | GATACTGTAG TTGCTCTCCA AGCTCTTGCC AAATATGCCA CTACCGCCTA |
| 51 | CATGCCATCT GAGGAGATCA ACCTGGTTGT AAAATCCACT GAGAATTTCC |
| 101 | AGCGCACATT CAACATACAG TCAGTTAACA GATTGGTATT TCAGCAGGAT |
| 151 | ACCCTGCCCA ATGTCCCTGG AATGTACACG TTGGAGGCCT CAGGCCAGGG |
| 201 | CTGTGTCTAT GTGCAG |
| SEQ ID NO: 100 (INSP088 protein sequence exon 16) | |
| 1 | DTVVALQALA KYATTAYMPS EEINLVVKST ENFQRTFNIQ SVNRLVFQQD |
| 51 | TLPNVPGMYT LEASGQGCVY VQ |
| SEQ ID NO: 101 (INSP088 nucleotide sequence exon 17) | |
| 1 | ACGGTGTTGA GATACAATAT TCTCCCTCCC ACAAATATGA AGACCTTTAG |
| 51 | TCTTAGTGTG GAAATAGGAA AAGCTAGATG TGAGCAACCG ACTTCACCTC |
| 101 | GATCCTTGAC TCTCACTATT CACACCAG |
| SEQ ID NO: 102 (INSP088 protein sequence exon 17) | |
| 1 | TVLRYNILPP TNMKTFSLSV EIGKARCEQP TSPRSLTLTI HTS |
| SEQ ID NO: 103 (INSP088 nucleotide sequence exon 18) | |
| 1 | TTATGTGGGG AGCCGTAGCT CTTCCAATAT GGCTATTGTG GAAGTGAAGA |
| 51 | TGCTATCTGG GTTCAGTCCC ATGGAGGGCA CCAATCAGTT A |
| SEQ ID NO: 104 (INSP088 protein sequence exon 18) | |
| 1 | YVGSRSSSNM AIVEVKMLSG FSPMEGTNQL |
| SEQ ID NO: 105 (INSP088 nucleotide sequence exon 19) | |
| 1 | CTTCTCCAGC AACCCCTGGT GAAGAAGGTT GAATTTGGAA CTGACACACT |
| 51 | TAACATTTAC TTGGATGAG |
| SEQ ID NO: 106 (INSP088 protein sequence exon 19) | |
| 1 | LLQQPLVKKV EFGTDTLNIY LDE |
| SEQ ID NO: 107 (INSP088 nucleotide sequence exon 20) | |
| 1 | CTCATTAAGA ACACTCAGAC TTACACCTTC ACCATCAGCC AAAGTGTGCT |
| 51 | GGTCACCAAC TTGAAACCAG CAACCATCAA GGTCTATGAC TACTACCTAC |
| 101 | CAG |
| SEQ ID NO: 108 (INSP088 protein sequence exon 20) | |
| 1 | LIKNTQTYTF TISQSVLVTN LKPATIKVYD YYLPD |
| SEQ ID NO: 109 (INSP088 nucleotide sequence exon 21) | |
| 1 | ATGAACAGGC AACAATTCAG TATTCTGATC CCTGTGAATG A |
| SEQ ID NO: 110 (INSP088 protein sequence exon 21) | |
| 1 | EQATIQYSDP CE |
| SEQ ID NO: 111 (INSP088 nucleotide sequence) | |
| 1 | CAGGTTTCCC TTGGCTTCTC CCCCTCCCAG CAGCTTCCAG GAGCAGAAGT |
| 51 | GGAGCTGCAG CTGCAGGCAG CTCCCGGATC CCTGTGTGCG CTCCGGGCGG |
| 101 | TGGATGAGAG TGTCTTACTG CTTAGGCCAG ACAGAGAGCT GAGCAACCGC |
| 151 | TCTGTCTATG GGATGTTTCC ATTCTGGTAT GGTCACTACC CCTATCAAGT |
| 201 | GGCTGAGTAT GATCAGTGTC CAGTGTCTGG CCCATGGGAC TTTCCTCAGC |
| 251 | CCCTCATTGA CCCAATGCCC CAAGGGCATT CGAGCCAGCG TTCCATTATC |
| 301 | TGGAGGCCCT CGTTCTCTGA AGGCACGGAC CTTTTCAGCT TTTTCCGGGA |
| 351 | CGTGGGCCTG AAAATACTGT CCAATGCCAA AATCAAGAAG CCAGTAGATT |
| 401 | GCAGTCACAG ATCTCCAGAA TACAGCACTG CTATGGGTGC AGGCGGTGGT |
| 451 | CATCCAGAGG CTTTTGAGTC ATCAACTCCT TTACATCAAG CAGAGGATTC |
| 501 | TCAGGTCCGC CAGTACTTCC CAGAGACCTG GCTCTGGGAT CTGTTTCCTA |
| 551 | TTGGTAACTC GGGGAAGGAG GCGGTCCACG TCACAGTTCC TGACGCCATC |
| 601 | ACCGAGTGGA AGGCGATGAG TTTCTGCACT TCCCAGTCAA GAGGCTTCGG |
| 651 | GCTTTCACCC ACTGTTGGAC TAACTGCTTT CAAGCCGTTC TTTGTTGACC |
| 701 | TGACTCTCCC TTACTCAGTA GTCCGTGGGG AATCCTTTCG TCTTACTGCC |
| 751 | ACCATCTTCA ATTACCTAAA GGATTGCATC AGGGTTCAGA CTGACCTGGC |
| 801 | TAAATCGCAT GAGTACCAGC TAGAATCATG GGCAGATTCT CAGACCTCCA |
| 851 | GTTGTCTCTG TGCTGATGAC GCAAAAACCC ACCACTGGAA CATCACAGCT |
| 901 | GTCAAATTGG GTCACATTAA CTTTACTATT AGTACAAAGA TTCTGGACAG |
| 951 | CAATGAACCA TGTGGGGGCC AGAAGGGGTT TGTTCCCCAA AAGGGCCGAA |
| 1001 | GTGACACGCT CATCAAGCCA GTTCTCGTCA AACCTGAGGG AGTCCTGGTG |
| 1051 | GAGAAGACAC ACAGCTCATT GCTGTGCCCA AAAGGAAAGG TGGCATCTGA |
| 1101 | ATCTGTCTCC CTGGAGCTCC CAGTGGACAT TGTTCCTGAC TCGACCAAGG |
| 1151 | CTTATGTTAC GGTTCTGGGA GACATTATGG GCACAGCCCT GCAGAACCTG |
| 1201 | GATGGTCTGG TGCAGATGCC CAGTGGCTGT GGCGAGCAGA ACATGGTCTT |
| 1251 | GTTTGCTCCC ATCATCTATG TCTTGCAGTA CCTGGAGAAG GCAGGGCTGC |
| 1301 | TGACGGAGGA GATCAGGTCT CGGGCAGTGG GTTTCCTGGA AATAGGGTAC |
| 1351 | CAGAAGGAGC TGATGTACAA ACACAGCAAT GGCTCATACA GTGCCTTTGG |
| 1401 | GGAGCGAGAT GGAAATGGAA ACACATGGCT GACAGCGTTT GTCACAAAAT |
| 1451 | GCTTTGGCCA AGCTCAGAAA TTCATCTTCA TTGATCCCAA GAACATCCAG |
| 1501 | GATGCTCTCA AGTGGATGGC AGGAAACCAG CTCCCCAGTG GCTGCTATGC |
| 1551 | CAACGTGGGA AATCTCCTTC ACACAGCTAT GAAGGGTGGT GTTGATGATG |
| 1601 | AGGTCTCCTT GACTGCGTAT GTCACAGCTG CATTGCTGGA GATGGGAAAG |
| 1651 | GATGTAGATG ACCCAATGGT GAGTCAGGGT CTACGGTGTC TCAAGAATTC |
| 1701 | GGCCACCTCC ACGACCAACC TCTACACACA GGCCCTGTTG GCTTACATTT |
| 1751 | TCTCCCTGGC TGGGGAAATG GACATCAGAA ACATTCTCCT TAAACAGTTA |
| 1801 | GATCAACAGG CTATCATCTC AGGAGAATCC ATTTACTGGA GCCAGAAACC |
| 1851 | TACTCCATCA TCGAACGCCA GCCCTTGGTC TGAGCCTGCG GCTGTAGATG |
| 1901 | TGGAACTCAC AGCATATGCA TTGTTGGCCC AGCTTACCAA GCCCAGCCTG |
| 1951 | ACTCAAAAGG AGATAGCGAA GGCCACTAGC ATAGTGGCTT GGTTGGCCAA |
| 2001 | GCAACACAAT GCATATGGGG GCTTCTCTTC TACTCAGGAT ACTGTAGTTG |
| 2051 | CTCTCCAAGC TCTTGCCAAA TATGCCACTA CCGCCTACAT GCCATCTGAG |
| 2101 | GAGATCAACC TGGTTGTAAA ATCCACTGAG AATTTCCAGC GCACATTCAA |
| 2151 | CATACAGTCA GTTAACAGAT TGGTATTTCA GCAGGATACC CTGCCCAATG |
| 2201 | TCCCTGGAAT GTACACGTTG GAGGCCTCAG GGCAGGGCTG TGTCTATGTG |
| 2251 | CAGACGGTGT TGAGATACAA TATTCTCCCT CCCACAAATA TGAAGACCTT |
| 2301 | TAGTCTTAGT GTGGAAATAG GAAAAGCTAG ATGTGAGCAA CCGACTTCAC |
| 2351 | CTCGATCCTT GACTCTCACT ATTCACACCA GTTATGTGGG GAGCCGTAGC |
| 2401 | TCTTCCAATA TGGCTATTGT GGAAGTGAAG ATGCTATCTG GGTTCAGTCC |
| 2451 | CATGGAGGGC ACCAATCAGT TACTTCTCCA GCAACCCCTG GTGAAGAAGG |
| 2501 | TTGAATTTGG AACTGACACA CTTAACATTT ACTTGGATGA GCTCATTAAG |
| 2551 | AACACTCAGA CTTACACCTT CACCATCAGC CAAAGTGTGC TGGTCACCAA |
| 2601 | CTTGAAACCA GCAACCATCA AGGTCTATGA CTACTACCTA CCAGATGAAC |
| 2651 | AGGCAACAAT TCAGTATTCT GATCCCTGTG AATGA |
| SEQ ID NO: 112 (INSP088 protein sequence) | |
| 1 | QVSLGFSPSQ QLPGAEVELQ LQAAPGSLCA LRAVDESVLL LRPDRELSNR |
| 51 | SVYGMFPFWY GHYPYQVAEY DQCPVSGPWD FPQPLIDPMP QGHSSQRSII |
| 101 | WRPSFSEGTD LFSFFRDVGL KILSNAKIKK PVDCSHRSPE YSTAMGAGGG |
| 151 | HPEAFESSTP LHQAEDSQVR QYFPETWLWD LFPIGNSGKE AVHVTVPDAI |
| 201 | TEWKAMSFCT SQSRGFGLSP TVGLTAFKPF FVDLTLPYSV VRGESFRLTA |
| 251 | TIFNYLKDCI RVQTDLAKSH EYQLESWADS QTSSCLCADD AKTHHWNITA |
| 301 | VKLGHINFTI STKILDSNEP CGGQKGFVPQ KGRSDTLIKP VLVKPEGVLV |
| 351 | EKTHSSLLCP KGKVASESVS LELPVDIVPD STKAYVTVLG DIMGTALQNL |
| 401 | DGLVQMPSGC GEQNMVLFAP IIYVLQYLEK AGLLTEEIRS RAVGFLEIGY |
| 451 | QKELMYKHSN GSYSAFGERD GNGNTWLTAF VTKCFGQAQK FIFIDPKNIQ |
| 501 | DALKWMAGNQ LPSGCYANVG NLLHTAMKGG VDDEVSLTAY VTAALLEMGK |
| 551 | DVDDPMVSQG LRCLKNSATS TTNLYTQALL AYIFSLAGEM DIRNILLKQL |
| 601 | DQQAIISGES IYWSQKPTPS SNASPWSEPA AVDVELTAYA LLAQLTKPSL |
| 651 | TQKEIAKATS IVAWLAKQHN AYGGFSSTQD TVVALQALAK YATTAYMPSE |
| 701 | EINLVVKSTE NFQRTFNIQS VNRLVFQQDT LPNVPGMYTL EASGQGCVYV |
| 751 | QTVLRYNILP PTNMKTFSLS VEIGKARCEQ PTSPRSLTLT IHTSYVGSRS |
| 801 | SSNMAIVEVK MLSGFSPMEG TNQLLLQQPL VKKVEFGTDT LNIYLDELIK |
| 851 | NTQTYTFTIS QSVLVTNLKP ATIKVYDYYL PDEQATIQYS DPCE |

SEQ ID NO:113 (INSP087 receptor binding domain protein
sequence)
MVVIDVKMLSGFTPTMSSIEELENKGQVMKTEVKNDHVLFYLENGFGRADSFPFSVEQSN
LVFNIQPAPAMVYDYYEKEEYALAFYNIDSSSVSQ
SEQ ID NO:114 (INSP087 receptor binding domain nucelotide
sequence)
ATGGTGGTTATAGATGTAAAAATGCTATCAGGATTTACTCCAACCATGTCATCCATTGAA
GAGCTTGAAAACAAGGGCCAAGTGATGAAGACTGAAGTCAAGAATGACCATGTTCTTTTC
TACTTGGAAAATGGTTTTGGTCGAGCAGACAGTTTCCCTTTTTCTGTTGAGCAGAGCAAC
CTTGTGTTCAACATTCAGCCAGCCCCAGCCATGGTCTACGATTATTACGAAAAAGAAGAA
TATGCCCTAGCTTTTTACAACATCGACAGTAGTTCAGTTTCCCAG
SEQ ID NO:115 (Cloned INSP087 receptor binding domain
protein sequence)
MVVIDVKMLSGFTPTMSSIEELENKGQVMKTEVKNDHVLFYLENGFGRADSFPFSVEQSN
LVFNIQPAPAMVYDYYEKEEYALAFYNIDSSSVSE
SEQ ID NO:116 (Cloned INSP087 receptor binding domain
nucleotide sequence)
ATGGTGGTTATAGATGTAAAAATGCTATCAGGATTTACTCCAACCATGTCATCCATTGAA
GAGCTTGAAAACAAGGGCCAAGTGATGAAGACTGAAGTCAAGAATGACCATGTTCTTTTC
TACTTGGAAAATGGTTTTGGTCGAGCAGACAGTTTCCCTTTTTCTGTTGAGCAGAGCAAC
CTTGTGTTCAACATTCAGCCAGCCCCAGCCATGGTCTACGATTACTATGAAAAAGAAGAA
TATGCCCTAGCTTTTTACAACATCGACAGTAGTTCAGTTTCCGAG
SEQ ID NO:117 (INSP088 receptor binding domain protein
sequence)
NMAIVEVKMLSGFSPMEGTNQLLLQQPLVKKVEFGTDTLNIYLDELIKNTQTYTFTISQS
VLVTNLKPATIKVYDYYLPDEQATIQYSDPCE
SEQ ID NO:118 (INSP088 receptor binding domain
nucleotide sequence)
AATATGGCTATTGTGGAAGTGAAGATGCTATCTGGGTTCAGTCCCATGGAGGGCACCAAT
CAGTTACTTCTCCAGCAACCCCTGGTGAAGAAGGTTGAATTTGGAACTGACACACTTAAC
ATTTACTTGGATGAGCTCATTAAGAACACTCAGACTTACACCTTCACCATCAGCCAAAGT
GTGCTGGTCACCAACTTGAAACCAGCAACCATCAAGGTCTATGACTACTACCTACCAGAT
GAACAGGCAACAATTCAGTATTCTGATCCCTGTGAA
SEQ ID NO:119 (INSP088 macroglobulin domain protein
sequence)
QAEDSQVRQYFPETWLWDLFPIGNSGKEAVHVTVPDAITEWKAMSFCTSQSRGFGLSPTV
GLTAFKPFFVDLTLPYSVVRGESFRLTATIFNYLKDCIRVQTDLAKSHEYQLESWADSQT
SSCLCADDAKTHHWNITAVKLGHINFTISTKILDSNEPCGGQKGFVPQKGRSDTLIKPVL
VKPEGVLVEKTHSSLLCPKGKVASESVSLELPVDIVPDSTKAYVTVLGDIMGTALQNLDG
LVQMPSGCGEQNMVLFAPIIYVLQYLEKAGLLTEEIRSRAVGFLEIGYQKELMYKHSNGS
YSAFGERDGNGNTWLTAFVTKCFGQAQKFIFIDPKNIQDALKWMAGNQLPSGCYANVGNL
LHTAMKGGVDDEVSLTAYVTAALLEMGKDVDDPMVSQGLRCLKNSATSTTNLYTQALLAY
IFSLAGEMDIRNILLKQLDQQAIISGESIYWSQKPTPSSNASPWSEPAAVDVELTAYALL
AQLTKPSLTQKEIAKATSIVAWLAKQHNAYGGFSSTQDTVVALQALAKYATTAYMPSEEI
NLVVKSTENFQRTFNIQSVNRLVFQQDTLPNVPGMYTLEASGQGCVYVQTVLRYNILPPT
NMKTFSLSVEIGKARCEQPTSPRSLTLTIHTSYVGSRSSSNMAIVEVKMLSGFSPMEGTN
QLLLQQPLVKKVEFGTDTLNIYLDELIKNTQTYTFTISQSVLVTNLKPATIKVYDYYLPD
EQATIQYSDPCE
SEQ ID NO:120 (INSP088 macroglobulin domain nucleotide
sequence)
CAAGCAGAGGATTCTCAGGTCCGCCAGTACTTCCCAGAGACCTGGCTCTGGGATCTGTTT
CCTATTGGTAACTCGGGGAAGGAGGCGGTCCACGTCACAGTTCCTGACGCCATCACCGAG
TGGAAGGCGATGAGTTTCTGCACTTCCCAGTCAAGAGGCTTCGGGCTTTCACCCACTGTT
GGACTAACTGCTTTCAAGCCGTTCTTTGTTGACCTGACTCTCCCTTACTCAGTAGTCCGT
GGGGAATCCTTTCGTCTTACTGCCACCATCTTCAATTACCTAAAGGATTGCATCAGGGTT
CAGACTGACCTGGCTAAATCGCATGAGTACCAGCTAGAATCATGGGCAGATTCTCAGACC
TCCAGTTGTCTCTGTGCTGATGACGCAAAAACCCACCACTGGAACATCACAGCTGTCAAA
TTGGGTCACATTAACTTTACTATTAGTACAAAGATTCTGGACAGCAATGAACCATGTGGG
GGCCAGAAGGGGTTTGTTCCCCAAAAGGGCCGAAGTGACACGCTCATCAAGCCAGTTCTC
GTCAAACCTGAGGGAGTCCTGGTGGAGAAGACACACAGCTCATTGCTGTGCCCAAAAGGA
AAGGTGGCATCTGAATCTGTCTCCCTGGAGCTCCCAGTGGACATTGTTCCTGACTCGACC
AAGGCTTATGTTACGGTTCTGGGAGACATTATGGGCAGAGCCCTGCAGAACCTGGATGGT
CTGGTGCAGATGCCCAGTGGCTGTGGCGAGCAGAACATGGTCTTGTTTGCTCCCATCATC
TATGTCTTGCAGTACCTGGAGAAGGCAGGGCTGCTGACGGAGGAGATCAGGTCTCGGGCA
GTGGGTTTCCTGGAAATAGGGTACCAGAAGGAGCTGATGTACAAACACAGCAATGGCTCA
TACAGTGCCTTTGGGGAGCGAGATGGAAATGGAAACACATGGCTGACAGCGTTTGTCACA
AAATGCTTTGGCCAAGCTCAGAAATTCATCTTCATTGATCCCAAGAACATCCAGGATGCT
CTCAAGTGGATGGCAGGAAACCAGCTCCCCAGTGGCTGCTATGCCAACGTGGGAAATCTC
CTTCACACAGCTATGAAGGGTGGTGTTGATGATGAGGTCTCCTTGACTGCGTATGTCACA
GCTGCATTGCTGGAGATGGGAAAGGATGTAGATGACCCAATGGTGAGTCAGGGTCTACGG
TGTCTCAAGAATTCGGCCACCTCCACGACCAACCTCTACACACAGGCCCTGTTGGCTTAC
ATTTTCTCCCTGGCTGGGGAAATGGACATCAGAAACATTCTCCTTAAACAGTTAGATCAA
CAGGCTATCATCTCAGGAGAATCCATTTACTGGAGCCAGAAACCTACTCCATCATCGAAC
GCCAGCCCTTGGTCTGAGCCTGCGGCTGTAGATGTGGAACTCACAGCATATGCATTGTTG
GCCCAGCTTACCAAGCCCAGCCTGACTCAAAAGGAGATAGCGAAGGCCACTAGCATAGTG
GCTTGGTTGGCCAAGCAACACAATGCATATGGGGGCTTCTCTTCTACTCAGGATACTGTA
GTTGCTCTCCAAGCTCTTGCCAAATATGCCACTACCGCCTACATGCCATCTGAGGAGATC
AACCTGGTTGTAAAATCCACTGAGAATTTCCAGCGCACATTCAACATACAGTCAGTTAAC
AGATTGGTATTTCAGCAGGATACCCTGCCCAATGTCCCTGGAATGTACACGTTGGAGGCC
TCAGGCCAGGGCTGTGTCTATGTGCAGACGGTGTTGAGATACAATATTCTCCCTCCCACA
AATATGAAGACCTTTAGTCTTAGTGTGGAAATAGGAAAAGCTAGATGTGAGCAACCGACT
TCACCTCGATCCTTGACTCTCACTATTCACACCAGTTATGTGGGGAGCCGTAGCTCTTCC
AATATGGCTATTGTGGAAGTGAAGATGCTATCTGGGTTCAGTCCCATGGAGGGCACCAAT
CAGTTACTTCTCCAGCAACCCCTGGTGAAGAAGGTTGAATTTGGAACTGACACACTTAAC
ATTTACTTGGATGAGCTCATTAAGAACACTCAGACTTACACCTTCACCATCAGCCAAAGT
GTGCTGGTCACCAACTTGAAACCAGCAACCATCAAGGTCTATGACTACTACCTACCAGAT
GAACAGGCAACAATTCAGTATTCTGATCCCTGTGAA
SEQ ID NO:121 (Cloned fragment of INSP088 - protein
sequence)
GHPEAFESSTPLHQAEDSQVRQYFPETWLWDLFPIGNSGKEAVHVTVPDAITEWKAMSFC
TSQSRGFGLSPTVGLTAFKPFFVDLTLPYSVVRGESFRLTATIFNYLKDCIRVQTDLAKS
HEYQLESWADSQTSSCLCADEAKTHHWNITAVKLGHINFTISTKILDSNEPCGGQKGFVP
QKGRSDTLIKPVLVKPEGVLVEKTHSSLLCPKGKVASESVSLELPVDIVPDSTKAYVTVL
GDIMGTALQNLDGLVQMPSGCGEQNMVLFAPIIYVLQYLEKAGLLTEEIRSRAVGFLEIG
YQKELMYKHSNGSYSAFGERDGNGNTWLTAFVTKCFGQAQKFIFIDPKNIQDALKWMAGN
QLPSGCYANVGNLLHTAMKGGVDDEVSLTAYVTAALLEMGKDVDDPMVSQGLWCLKNSAT
STTNLYTQALLAYIFSLAGEMDIRNILLKQLDQQAIISGESIYWSQKPTPSSNASPWSEP
AAVDVELTAYALLAQLTKPSLTQKEIAKATSIVAWLAKQRNAYGGFSSTQDTVVALQALA
KYATTAYVPSEEINLVVKSTENFQRTFNIQSVNRLVFQQDTLPNVPGMYTLEASGQGCVY
VQTVLRYNILPPTNMKTFSLSVEIGKARCEQPTSPRSLTLTIHTSYVGSRSSSNMAIVEV
KMLSGFSPMEGTNQLLLQQPLVKKVEFGTDTLNIYLDELIKNTQTYTFTISQSVLVTNLK
PATIKVYDYYLPDEQATIQYSDPCE
SEQ ID NO: 122 (Cloned fragment of INSP088 - nucleotide
sequence)
GTGGTCATCCAGAGGCTTTTGAGTCATCAACTCCTTTACATCAAGCAGAGGATTCTCAGG
TCCGCCAGTACTTCCCAGAGACCTGGCTCTGGGATCTGTTTCCTATTGGTAACTCGGGGA
AGGAGGCGGTCCACGTCACAGTTCCTGACGCCATCACCGAGTGGAAGGCGATGAGTTTCT
GCACTTCCCAGTCAAGAGGCTTCGGGCTTTCACCCACTGTTGGACTAACTGCTTTCAAGC
CATTCTTTGTTGACCTGACTCTCCCTTACTCAGTAGTCCGTGGGGAATCCTTTCGTCTTA
CTGCCACCATCTTCAATTACCTAAAGGATTGCATCAGGGTTCAGACTGACCTGGCTAAAT
CGCATGAGTACCAGCTAGAATCATGGGCAGATTCTCAGACCTCCAGTTGTCTCTGTGCTG
ATGAAGCAAAAACCCACCACTGGAACATCACAGCTGTCAAATTGGGTCACATTAACTTTA
CTATTAGTACAAAGATTCTGGACAGCAATGAACCATGTGGGGGCCAGAAGGGGTTTGTTC
CCCAAAAGGGCCGAAGTGACACGCTCATCAAGCCAGTTCTCGTCAAACCTGAGGGAGTCC
TGGTGGAGAAGACACACAGCTCATTGCTGTGCCCAAAAGGAAAGGTGGCATCTGAATCTG
TCTCCCTGGAGCTCCCAGTGGACATTGTTCCTGACTCGACCAAGGCTTATGTTACGGTTC
TGGGAGACATTATGGGCACAGCCCTGCAGAACCTGGATGGTCTGGTGCAGATGCCCAGTG
GCTGTGGCGAGCAGAACATGGTCTTGTTTGCTCCCATCATCTATGTCTTGCAGTACCTGG
AGAAGGCAGGGCTGCTGACGGAGGAGATCAGGTCTCGGGCAGTGGGTTTCCTGGAAATAG
GGTACCAGAAGGAGCTGATGTACAAACACAGCAATGGCTCATACAGTGCCTTTGGGGAGC
GAGATGGAAATGGAAACACATGGCTGACAGCGTTTGTCACAAAATGCTTTGGCCAAGCTC
AGAAATTCATCTTCATTGATCCCAAGAACATCCAGGATGCTCTCAAGTGGATGGCAGGAA
ACCAGCTCCCCAGTGGCTGCTATGCCAACGTGGGAAATCTCCTTCACACAGCTATGAAGG
GTGGTGTTGATGATGAGGTCTCCTTGACTGCGTATGTCACAGCTGCATTGCTGGAGATGG
GAAAGGATGTAGATGACCCAATGGTGAGTCAGGGTCTATGGTGTCTCAAGAATTCGGCCA
CCTCCACGACCAACCTCTACACACAGGCCCTGTTGGCTTACATTTTCTCCCTGGCTGGGG
AAATGGACATCAGAAACATTCTCCTTAAACAGTTAGATCAACAGGCTATCATCTCAGGAG
AATCCATTTACTGGAGCCAGAAACCTACTCCATCATCGAACGCCAGCCCTTGGTCTGAGC
CTGCGGCTGTAGATGTGGAACTCACAGCATATGCATTGTTGGCCCAGCTTACCAAGCCCA
GCCTGACTCAAAAGGAGATAGCGAAGGCCACTAGCATAGTGGCTTGGTTGGCCAAGCAAC
GCAATGCATATGGGGGCTTCTCTTCTACTCAGGATACTGTAGTTGCTCTCCAAGCTCTTG
CCAAATATGCCACTACCGCCTACGTGCCATCTGAGGAGATCAACCTGGTTGTAAAATCCA
CTGAGAATTTCCAGCGCACATTCAACATACAGTCAGTTAACAGATTGGTATTTCAGCAGG
ATACCCTGCCCAATGTCCCTGGAATGTACACGTTGGAGGCCTCAGGCCAGGGCTGTGTCT
ATGTGCAGACGGTGTTGAGATACAATATTCTCCCTCCCACAAATATGAAGACCTTTAGTC
TTAGTGTGGAAATAGGAAAAGCTAGATGTGAGCAACCGACTTCACCTCGATCCTTGACTC
TCACTATTCACACCAGTTATGTGGGGAGCCGTAGCTCTTCCAATATGGCTATTGTGGAAG
TGAAGATGCTATCTGGGTTCAGTCCCATGGAGGGCACCAATCAGTTACTTCTCCAGCAAC
CCCTGGTGAAGAAGGTTGAATTTGGAACTGACACACTTAACATTTACTTGGATGAGCTCA
TTAAGAACACTCAGACTTACACCTTCACCATCAGCCAAAGTGTGCTGGTCACCAACTTGA
AACCAGCAACCATCAAGGTCTATGACTACTACCTACCAGATGAACAGCCAACAATTCAGT
ATTCTGATCCCTGTGAA

1-49. (canceled)

50. A composition of matter comprising:
a) an isolated polypeptide selected from the group consisting of:
1) an amino acid sequence comprising that recited in SEQ ID NO:68 or SEQ ID NO:112;
2) a fragment of said amino acid sequence which functions as an alpha-2-macroglobulin-like proteinase inhibitor, or has an antigenic determinant in common with a polypeptide according to 1);
3) a functional equivalent of 1) or 2);
4) an amino acid sequence consisting of that recited in SEQ ID NO:68 or SEQ ID NO:112;
5) the fragment of 2), wherein the fragment comprises the amino acid sequence of SEQ ID NO:113 or SEQ ID NO:115, or a functional equivalent thereof;
6) the fragment of 2), wherein the fragment consists of the amino acid sequence of SEQ ID NO:113 or SEQ ID NO:115, or a functional equivalent thereof;
7) the fragment of 2), wherein the fragment comprises an amino acid sequence selected from the group consisting of: SEQ ID NO:117, SEQ ID NO:119, and SEQ ID NO:121, or a functional equivalent of any of the foregoing;
8) the fragment of 2), wherein the fragment consists of an amino acid sequence selected from the group consisting of: SEQ ID NO:117, SEQ ID NO:119, and SEQ ID NO:121, or a functional equivalent of any of the foregoing;
9) the functional equivalent of 3), wherein the functional equivalent is homologous to the amino acid sequence recited in SEQ ID NO:68 or SEQ ID NO:112, and wherein the functional equivalent is an alpha-2-macroglobulin-like proteinase inhibitor;
10) the functional equivalent of 3), wherein the functional equivalent is an alpha-2-macroglobulin-like proteinase inhibitor, and wherein the functional equivalent has greater than 80% sequence identity with the amino acid sequence recited in SEQ ID NO:68 or SEQ ID NO:112, or with an active fragment thereof;
11) the functional equivalent of 3), wherein the functional equivalent exhibits significant structural homology with a polypeptide comprising the amino acid sequence recited in SEQ ID NO:68 or SEQ ID NO:112;
12) the fragment of 2), wherein the fragment has an antigenic determinant in common with the amino acid sequence of 1), and wherein the antigenic determinant consists of at least 7 amino acid residues from the amino acid sequence recited in SEQ ID NO:68 or SEQ ID NO:112;
b) a purified nucleic acid molecule:
1) encoding a polypeptide of any of a1) to a12);
2) comprising the nucleic acid sequence recited in SEQ ID NO:67 or SEQ ID NO:111, or a redundant equivalent or fragment thereof;
3) consisting of the nucleic acid sequence recited in SEQ ID NO:67 or SEQ ID NO:111, or a redundant equivalent or fragment thereof;
4) comprising a nucleic acid sequence selected from the group consisting of SEQ ID NO:114, SEQ ID NO:116, SEQ ID NO:118, SEQ ID NO:120, and SEQ ID NO:122, or a redundant equivalent of any of the foregoing;
5) consisting of a nucleic acid sequence selected from the group consisting of SEQ ID NO:114, SEQ ID NO:116, SEQ ID NO:118, SEQ ID NO:120, and SEQ ID NO:122, or a redundant equivalent of any of the foregoing;
6) that hybridizes under high stringency conditions with a nucleic acid molecule of any of b1) to b5);
c) a vector comprising a nucleic acid molecule according to any one of b1) to b6);
d) a host cell transformed with a vector or a nucleic acid molecule according to any one of b) or c);
e) a ligand:
1) that binds specifically to the polypeptide of any of a1) to a12);
2) which is an antibody that binds specifically to the polypeptide of any of a1) to a12);
f) a compound:
1) that increases the level of expression or activity of a polypeptide according to any of a1) to a12);
2) that decreases the level of expression or activity of a polypeptide according to any of a1) to a12);
g) a compound that binds to a polypeptide according to any of a1) to a12) without inducing any of the biological effects of the polypeptide;
h) a compound that binds to a polypeptide according to any of a1) to a12) without inducing any of the biological effects of the polypeptide, wherein the compound is a natural or modified substrate, ligand, enzyme, receptor or structural or functional mimetic;
i) a pharmaceutical composition comprising any one of a) to h), and a pharmaceutically acceptable carrier;
j) a vaccine composition comprising any one of a1) to a12) or b1) to b6);
k) a kit for diagnosing disease, comprising a first container containing a nucleic acid probe that hybridizes under stringent conditions with a nucleic acid molecule of any one of b1) to b6), a second container containing primers useful for amplifying the nucleic acid molecule, and instructions for using the probe and primers for facilitating the diagnosis of disease;
l) a kit for diagnosing disease, comprising a first container containing a nucleic acid probe that hybridizes under stringent conditions with a nucleic acid molecule of any one of b1) to b6); a second container containing primers useful for amplifying the nucleic acid molecule; a third container holding an agent for digesting unhybridized RNA; and instructions for using the probe and primers for facilitating the diagnosis of disease;
m) a kit comprising an array of nucleic acid molecules, at least one of which is a nucleic acid molecule according to any one of b1) to b6);
n) a kit comprising one or more antibodies that bind to a polypeptide as recited in any one of a1) to a12); and a reagent useful for the detection of a binding reaction between the one or more antibodies and the polypeptide; or
o) a transgenic or knockout non-human animal that has been transformed to express higher, lower, or absent levels of a polypeptide according to any one of a1) to a12).

51. A method of using a composition of matter, comprising obtaining a composition of matter according to claim 50 and using said composition of matter in a method selected from: diagnosing a disease in a patient; treatment of a disease in a patient; monitoring the therapeutic treatment of a disease; identification of a compound that is effective in the treatment and/or diagnosis of a disease; and screening candidate compounds.

52. The method of claim 51, wherein said method of using a composition of matter comprises the method for treatment of a disease, comprising administering to the patient a polypeptide selected from the group consisting of:
a) an amino acid sequence comprising that recited in SEQ ID NO:68 or SEQ ID NO:112;
b) a fragment of said amino acid sequence which functions as an alpha-2-macroglobulin-like proteinase inhibitor, or has an antigenic determinant in common with a polypeptide according to a);
c) a functional equivalent of a) or b);
d) an amino acid sequence consisting of that recited in SEQ ID NO:68 or SEQ ID NO:112;
e) the fragment of b), wherein the fragment comprises the amino acid sequence of SEQ ID NO:113 or SEQ ID NO:115, or a functional equivalent thereof;
f) the fragment of b), wherein the fragment consists of the amino acid sequence of SEQ ID NO:113 or SEQ ID NO:115, or a functional equivalent thereof;
g) the fragment of b), wherein the fragment comprises an amino acid sequence selected from the group consisting of: SEQ ID NO:117, SEQ ID NO:119, and SEQ ID NO:121, or a functional equivalent of any of the foregoing;
h) the fragment of b), wherein the fragment consists of an amino acid sequence selected from the group consisting of: SEQ ID NO:117, SEQ ID NO:119, and SEQ ID NO:121, or a functional equivalent of any of the foregoing;
i) the functional equivalent of c), wherein the functional equivalent is homologous to the amino acid sequence recited in SEQ ID NO:68 or SEQ ID NO:112, and wherein the functional equivalent is an alpha-2-macroglobulin-like proteinase inhibitor;
j) the functional equivalent of c), wherein the functional equivalent is an alpha-2-macroglobulin-like proteinase inhibitor, and wherein the functional equivalent has greater than 80% sequence identity with the amino acid sequence recited in SEQ ID NO:68 or SEQ ID NO:112, or with an active fragment thereof;
k) the functional equivalent of c), wherein the functional equivalent exhibits significant structural homology with a polypeptide comprising the amino acid sequence recited in SEQ ID NO:68 or SEQ ID NO:112; and
l) the fragment of b), wherein the fragment has an antigenic determinant in common with the amino acid sequence of a), and wherein the antigenic determinant consists of at least 7 amino acid residues from the amino acid sequence recited in SEQ ID NO:68 or SEQ ID NO:112.

53. The method of claim 51, wherein said method of using a composition of matter comprises the method for treatment of a disease, comprising administering to the patient a nucleic acid molecule:
a) encoding a polypeptide selected from the group consisting of: an amino acid sequence comprising that recited in SEQ ID NO:68 or SEQ ID NO:112; a fragment of said amino acid sequence which functions as an alpha-2-macroglobulin-like proteinase inhibitor, or has an antigenic determinant in common with the polypeptide; a functional equivalent of the amino acid sequence or the fragment; an amino acid sequence consisting of that recited in SEQ ID NO:68 or SEQ ID NO:112; a fragment comprising the amino acid sequence of SEQ ID NO:113 or SEQ ID NO:115, or a functional equivalent thereof; a fragment consisting of the amino acid sequence of SEQ ID NO:113 or SEQ ID NO:115, or a functional equivalent thereof; a fragment comprising an amino acid sequence selected from the group consisting of: SEQ ID NO:117, SEQ ID NO:119, and SEQ ID NO:121, or a functional equivalent of any of the foregoing; a fragment consisting of an amino acid sequence selected from the group consisting of: SEQ ID NO:117, SEQ ID NO:119, and SEQ ID NO:121, or a functional equivalent of any of the foregoing; a functional equivalent of SEQ ID NO:68 or SEQ ID NO:112, wherein the functional equivalent is homologous to the amino acid sequence recited in SEQ ID NO:68 or SEQ ID NO:112, and wherein the functional equivalent is an alpha-2-macroglobulin-like proteinase inhibitor; a functional equivalent of SEQ ID NO:68 or SEQ ID NO:112, wherein the functional equivalent is an alpha-2-macroglobulin-like proteinase inhibitor, and wherein the functional equivalent has greater than 80% sequence identity with the amino acid sequence recited in SEQ ID NO:68 or SEQ ID NO:112, or with an active fragment thereof; a functional equivalent SEQ ID NO:68 or SEQ ID NO:112, wherein the functional equivalent exhibits significant structural homology with a polypeptide comprising the amino acid sequence recited in SEQ ID NO:68 or SEQ ID NO:112; and a fragment of SEQ ID NO:68 or SEQ ID NO:112, wherein the fragment has an antigenic determinant in common with the amino acid sequence of 1), and wherein the antigenic determinant consists of at least 7 amino acid residues from the amino acid sequence recited in SEQ ID NO:68 or SEQ ID NO:112;
b) comprising the nucleic acid sequence recited in SEQ ID NO:67 or SEQ ID NO:111, or a redundant equivalent or fragment thereof;
c) consisting of the nucleic acid sequence recited in SEQ ID NO:67 or SEQ ID NO:11, or a redundant equivalent or fragment thereof;
d) comprising a nucleic acid sequence selected from the group consisting of SEQ ID NO:114, SEQ ID NO:116, SEQ ID NO:118, SEQ ID NO:120, and SEQ ID NO:122, or a redundant equivalent of any of the foregoing;
e) consisting of a nucleic acid sequence selected from the group consisting of SEQ ID NO:114, SEQ ID NO:116, SEQ ID NO:118, SEQ ID NO:120, and SEQ ID NO:122, or a redundant equivalent of any of the foregoing; or
f) that hybridizes under high stringency conditions with a nucleic acid molecule of any of a) to e).

54-58. (canceled)

59. The method of claim 51, wherein said method of using a composition of matter comprises the method for treatment of a disease, and wherein the disease includes reproductive disorders, cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, pancreatitis, arthritis, psoriasis, psoriasis vulgaris, respiratory tract inflammation, asthma, and organ transplant rejection; cardiovascular disorders, including hypertension, oedema, angina, atherosclerosis, thrombosis, sepsis, shock, reperfusion injury, and ischemia, particularly ischemic heart disease; neurological disorders including central nervous system disease, Alzheimer's disease, brain injury, Parkinson's disease, amyotrophic lateral sclerosis, and pain; developmental disorders; metabolic disorders including diabetes mellitus, osteoporosis, and obesity, AIDS, renal disease, particularly idiopathic nephrotic syndrome; lung injury; infections including viral infection, bacterial infection, fungal infection and parasitic infection, particularly Trypanosoma cruzi infection and other pathological conditions.

60. The method of claim 51, wherein said method of using a composition of matter comprises the method for treatment of a disease, and wherein the disease is one for which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

61. The method of claim 51, wherein said method of using a composition of matter comprises the method for treatment of a disease, and wherein the disease is one for which expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

62. (canceled)

63. The method of claim 62, which is carried out in vitro.

64-67. (canceled)

68. The method of claim 67, further comprising amplifying the nucleic acid molecule to form an amplified product and detecting the presence or absence of a mutation in the amplified product.

69. The method of claim 67, wherein the presence or absence of the mutation in the patient is detected by contacting said nucleic acid molecule with a nucleic acid probe that hybridises to said nucleic acid molecule under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation.

70. The method of claim 62, wherein said disease includes, but is not limited to reproductive disorders, cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, pancreatitis, arthritis, psoriasis, psoriasis vulgaris, respiratory tract inflammation, asthma, and organ transplant rejection; cardiovascular disorders, including hypertension, oedema, angina, atherosclerosis, thrombosis, sepsis, shock, reperfusion injury, and ischemia, particularly ischemic heart disease; neurological disorders including central nervous system disease, Alzheimer's disease, brain injury, Parkinson's disease, amyotrophic lateral sclerosis, and pain; developmental disorders; metabolic disorders including diabetes mellitus, osteoporosis, and obesity, AIDS, renal disease, particularly idiopathic nephrotic syndrome; lung injury; infections including viral infection, bacterial infection, fungal infection and parasitic infection, particularly Trypanosoma cruzi infection and other pathological conditions.

71. The method of claim 62, wherein said disease is a disease in which alpha-2-macroglobulin-like proteinase inhibitors are implicated.

72-74. (canceled)

75. An isolated polypeptide comprising:
a) the amino acid sequence recited in SEQ ID NO:68 or SEQ ID NO:112;
b) a fragment thereof which functions as an alpha-2-macroglobulin-like proteinase inhibitor, or has an antigenic determinant in common with a polypeptide according to a); or
c) a functional equivalent of a) or b).

76. The isolated polypeptide of claim 75, wherein the polypeptide is a fragment comprising the amino acid sequence recited in SEQ ID NO:113 or SEQ ID NO:115, or a functional equivalent thereof.

77. The isolated polypeptide of claim 75, wherein the polypeptide is a fragment comprising an amino acid sequence selected from the group consisting of SEQ ID NO:117, SEQ ID NO:119, and SEQ ID NO:121, or a functional equivalent thereof.
